SEQUENCE LISTING (25 sequences)

SEQ ID NO: 1 - DNA, 597 bp, Homo sapiens
atgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatgtcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacgattcttctactcaaaacaaaagagccaacagaacagaagaaaatgtttcagacggttccccaaatgccggttctgtggagcagacgcccaagaagcctggcctcagaagacgtcaaacgtaa

SEQ ID NO: 2 - protein, 198 aa, Homo sapiens
MSNVRVSNGSPSLERMDARQAEHPKPSACRNLFGPVDHEELTRDLEKHCRDMEEASQRKWNFDFQNHKPLEGKYEWQEVEKGSLPEFYYRPPRPPKGACKVPAQESQDVSGSRPAAPLIGAPANSEDTHLVDPKTDPSDSQTGLAEQCAGIRKRPATDDSSTQNKRANRTEENVSDGSPNAGSVEQTPKKPGLRRRQT

SEQ ID NO: 3 - DNA, 495 bp, Homo sapiens
atgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaa

SEQ ID NO: 4 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 5 - DNA, 951 bp, Homo sapiens
atgtccgacgcgtccctccgcagcacatccacgatggagcgtcttgtcgcccgtgggaccttcccagtactagtgcgcaccagcgcctgccgcagcctcttcgggccggtggaccacgaggagctgagccgcgagctgcaggcccgcctggccgagctgaacgccgaggaccagaaccgctgggattacgacttccagcaggacatgccgctgcggggccctggacgcctgcagtggaccgaagtggacagcgactcggtgcccgcgttctaccgcgagacggtgcaggtggggcgctgccgcctgctgctggcgccgcggcccgtcgcggtcgcggtggctgtcagcccgcccctcgagccggccgctgagtccctcgacggcctcgaggaggcgccggagcagctgcctagtgtcccggtcccggccccggcgtccaccccgcccccagtcccggtcctggctccagccccggccccggctccggctccggtcgcggctccggtcgcggctccggtcgcggtcgcggtcctggccccggccccggccccggccccggctccggctccggccccggctccagtcgcggccccggccccagccccggccccggccccggccccggcccccgccccggccccggccccggacgcggcgcctcaagagagcgccgagcagggcgcgaaccaggggcagcgcggccaggagcctctcgctgaccagctgcactcggggatttcgggacgtcccgcggccggcaccgcggccgccagcgccaacggcgcggcgatcaagaagctgtccgggcctctgatctccgatttcttcgccaagcgcaagagatcagcgcctgagaagtcgtcgggcgatgtccccgcgccgtgtccctctccaagcgccgcccctggcgtgggctcggtggagcagaccccgcgcaagaggctgcggtga

SEQ ID NO: 6 - protein, 316 aa, Homo sapiens
MSDASLRSTSTMERLVARGTFPVLVRTSACRSLFGPVDHEELSRELQARLAELNAEDQNRWDYDFQQDMPLRGPGRLQWTEVDSDSVPAFYRETVQVGRCRLLLAPRPVAVAVAVSPPLEPAAESLDGLEEAPEQLPSVPVPAPASTPPPVPVLAPAPAPAPAPVAAPVAAPVAVAVLAPAPAPAPAPAPAPAPVAAPAPAPAPAPAPAPAPAPAPDAAPQESAEQGANQGQRGQEPLADQLHSGISGRPAAGTAAASANGAAIKKLSGPLISDFFAKRKRSAPEKSSGDVPAPCPSPSAAPGVGSVEQTPRKRLR

SEQ ID NO: 7 - DNA, 29 bp, Homo sapiens
ggatccgggagacatatgtcaaacgtgcg

SEQ ID NO: 8 - DNA, 34 bp, Homo sapiens
ggagtcttctgcagtttgcattactatccctagg

SEQ ID NO: 9 - DNA, 32 bp, Homo sapiens
gcttgcccgagttctatttcagacccccgcgg

SEQ ID NO: 10 - DNA, 32 bp, Homo sapiens
ccgcgggggtctgaaatagaactcgggcaagc

SEQ ID NO: 11 - DNA, 34 bp, Homo sapiens
gcttgcccgagttcttctacagacccccgcggcc

SEQ ID NO: 12 - DNA, 34 bp, Homo sapiens
ggccgcgggggtctgtagaagaactcgggcaagc

SEQ ID NO: 13 - DNA, 30 bp, Homo sapiens
cccctagagggcaagttcgagtggcaagag

SEQ ID NO: 14 - DNA, 30 bp, Homo sapiens
ctcttgccactcgaacttgccctctagggg

SEQ ID NO: 15 - DNA, 27 bp, Homo sapiens
gccgcgggggtctgaagaagaactcgg

SEQ ID NO: 16 - DNA, 24 bp, Homo sapiens
catatgcccaacctttttgtggca

SEQ ID NO: 17 - DNA, 25 bp, Homo sapiens
catatggtcaacagcctggagaaac

SEQ ID NO: 18 - DNA, 24 bp, Homo sapiens
cttcagcaggttctggtcttggtg

SEQ ID NO: 19 - DNA, 21 bp, Homo sapiens
gcatatggggcagcagcctgg

SEQ ID NO: 20 - DNA, 20 bp, Homo sapiens
gcaggcggatccgacgggcc

SEQ ID NO: 21 - protein, 15 aa, Homo sapiens
EKGSLPEFYYRPPRP

SEQ ID NO: 22 - protein, 12 aa, Homo sapiens
SLPEFYYRPPRP

SEQ ID NO: 23 - protein, 10 aa, Homo sapiens
LPEFYYRPPR

SEQ ID NO: 24 - protein, 8 aa, Homo sapiens
PEFYYRPP

SEQ ID NO: 25 - protein, 6 aa, Homo sapiens
EFYYRP

(19) United States
(12) Patent Application Publication
(10) Pub. No.: US 2008/0026992 A1
(43) Pub. Date: Jan. 31, 2008

(54) Tyrosine Phosphorylation of Cdk Inhibitor Proteins of the Cip/Kip Family

(76) Inventors: Ludger Hengst, Innsbruck (AT); Zoran Cilensek, The Hague (NL); Matthias Grimmier, Wurzburg (DE)

Correspondence Address: WILSON SONSINI GOODRICH & ROSATI, 650 PAGE MILL ROAD, PALO ALTO, CA, 94304-1050, US

(21) Appl. No.: 10/576,845

(22) PCT Filed: Oct. 20, 2004
(86) PCT No.: PCT/EP04/11860

§ 371 Date: Jun. 22, 2007

(30) Foreign Application Priority Data
Oct. 20, 2003 (EP) 03024165.7

(51) Int. Cl. A61K 38/00; A61P 43/00; C07K 14/00; C07K 16/18; C07K 7/00; C12N 15/00; C12N 15/11; C12N 5/06; C12N 7/00; G01N 33/53

(52) U.S. Cl. 514/12; 435/235.1; 435/320.1; 435/325; 435/331; 435/7.23; 530/300; 530/326; 530/327; 530/328; 530/350; 530/387.9; 530/388.1; 530/389.1; 536/23.1

(57) ABSTRACT

The present invention is directed to tyrosine phosphorylated forms of Cip/Kip proteins including p27Kip1, fragments of these forms and antibodies thereto. The invention is further related to non-phosphorylatable forms of Cip/Kip proteins, or fragments thereof. Further embodiments are diagnostic or therapeutic uses of the disclosed compounds, in particular uses in diagnostics and therapy of hyperproliferative diseases.

### FIELD OF THE INVENTION

The present invention is directed to tyrosine phosphorylated forms of p27Kip1, p21 Cip1 and p57 Kip2, fragments of these forms and antibodies thereto. The invention is further related to non-phosphorylatable mutant forms of p27Kip1, p21 Cip1 and p57 Kip2 or fragments thereof. Further embodiments are diagnostic or therapeutic uses of the disclosed compounds, in particular uses in diagnostics and therapy of hyperproliferative diseases.

### BACKGROUND OF THE INVENTION

Each cell division requires that all DNA, the centrosome and all other cellular components are replicated once per cell cycle and properly segregated into the newborn daughter cells. This is part of a strictly coordinated process of successive events which is referred to as the cell cycle. The duplication of the genetic information and its segregation into two daughter cells may be regarded as central processes of the cell cycle. Both events are separated from one another in higher eukaryotic cells (Howard, A., and Pelc, S. R., Exp. Cell Res. 2 (1951) 178-187). The duplication of the chromosomes occurs in the synthesis or S phase, the separation and segregation of the two sister chromatids into two daughter nuclei occurs during mitosis and cytokinesis. Both phases, mitosis/cytokinesis and DNA replication, are separated from one another by so-called gap phases: The phase before the synthesis phase is referred to as the G1 phase and the phase before mitosis is referred to as the G2 phase.

Cyclin-dependent kinases (CDKs) are the master regulators of the cell cycle control system. The amount and activity of these kinases ensures an orderly and uninterrupted progression through the cell cycle (Ekholm, S. V., and Reed, S. I., Curr. Opin. Cell Biol. 12 (2000) 676-684; Morgan, D. O., Annu. Rev. Cell. Dev. Biol. 13 (1997); Pines, J., and Rieder, C. L., Nat Cell Biol. 3 (2001) E3-6; Planas-Silva, M. D., and Weinberg, R. A., Curr. Opin. Cell Biol. 9 (1997) 768-772; Sherr, C. J., Science 274 (1996) 1672-1677). Specific linases are activated and inactivated in a phase-specific manner during the course of the cell cycle. Oscillating CDK activity is a prerequisite for progression through the cell cycle. Endogenous and internal checkpoints may regulate CDK kinase activity upon negative signals, for example due to lack of growth factors and these signals become thereby integrated into the cell cycle control system.

Monomeric CDK subunits are catalytically inactive and have to associate with a positive regulatory subunit of the cyclin protein family in order to become activated. The abundance of most cell cycle regulatory cyclins is subjected to major variations during progression through the cell cycle. These oscillations of the cyclins plays one major role in the stage-specific activation and deactivation of CDKs. CDK kinase activity is further regulated by inhibitory and activating phosphorylation events and through CDK inhibitory proteins (CKIs).

CKIs of mammals are divided into two families according to their structure and their mechanism of action i.e. the Cip/Kip and the INK4 family (Carnero, A., and Hannon, G. J., Curr. Top. Microbiol. Immunol. 227 (1998) 43-55; Hengst, L., and Reed, S. I., Curr. Top. Microbiol. Immunol. 227 (1998) 25-41). The members of the Cip/Kip family, p21^{Cip1}, p27^{Kip1} and p57^{Kip2} bind and inhibit a broad spectrum of cyclin/CDK complexes. They share a conserved amino-terminal domain which is necessary and sufficient for the inhibition of most cyclin/CDK complexes. Interestingly despite inhibiting CDKs, Cip/Kip proteins are suggested to act as activators of cyclin D/CDK4,6 kinase complexes (LaBaer, J., et al., Genes Dev. 11 (1997) 847-862; Cheng, M., et al., EMBO J. 18 (1999) 1571-1583). It is believed that they stimulate assembly of these complexes by binding to both subunits. Therefore, it has been reported that p21, p27and p57 can associate with cyclin D/CDK4 complexes without inactivating them, however a number of studies have also reported inactivation of CDK4 kinase by p21 and p27.

The three-dimensional structures of either a complex between CDK2 and truncated cyclin A or the ternary complex including the inhibitory domain of p27have been determined by X-ray crystallography (Russo, A. A., et al., Nature 382 (1996) 325-331) and allow inferences to be made about the mechanism of inhibition: The amino-terminal region of the inhibitor domain of p27 binds the conserved cyclin box of cyclin A without significantly impairing its rigid structure. The carboxy-terminal region of the inhibitor domain interacts with the amino-terminal domain of CDK2 and disturbs the conformation of its active site. In addition the inhibitor protrudes into the active centre of the kinase and thus blocks its ATP binding site. Even though the X-ray structure of p27 suggests that binding of p27 always leads to inactivation of the kinase complex, there are paradoxical observations that suggest that p27 or the related inhibitor p21 are required for activation of cyclin D/CDK4 complexes (LaBaer, J., et al., Genes Dev. 11 (1997) 847-862; Cheng, M., et al., EMBO J. 18 (1999) 1571-1583).

The carboxy-terminal domains of the Cip/Kip proteins are different in size and only have slight sequence homologies between one another. The activity of the Cip/Kip proteins can be specifically regulated by modifications and protein-protein inter-actions of these carboxy-terminal domains (Hengst, L., and Reed, S. I., Curr. Top. Microbiol. Immunol. 227 (1998) 25-41). For example phosphorylation of threonine 187 at the carboxy-terminal end has a major effect on the stability of p27 (Montagnoli, A., et al., Genes Dev. 13 (1999) 1181-1189; Sheaff, R. J., et al., Genes Dev. 11 (1997) 1464-1478). In addition p27 has a nuclear localization signal (NLS) in this region (Reynisdottir, I., and Massague, J., Genes Dev. 11 (1997) 492-503; Zeng Y., et al., Biochem. Biophys. Res. Commun. 274 (2000) 37-42). A comparable NLS in the C-terminal domains of p21 and p57 is also responsible for their nuclear location.

p21 and p27 have been characterized as ubiquitous negative regulators of cell proliferation, since a number of endogenous and exogenous antiproliferative signals results in their increased expression in many different cell types. p21 is often involved in checkpoint controls, stress response and in the induction of differentiation processes, whereas p27 seems to play a central role the control of the restriction point and G1/S transition.

p27^{Kip1} was discovered almost at the same time by several groups as a CDK-inhibiting activity in G1-arrested cell cultures (Hengst, L., et al., Proc. Natl. Acad. Sci. USA 91 (1994) 5291-5295; Polyak, K, et al., Genes Dev. 8 (1994) 9-22; Polyak, K, et al., Cell 78 (1994) 59-66; Slingerland, J. M., et al, Mol. Cell. Biol. 14 (1994) 3683-3694). Furthermore p27 was identified by a genetic screen as a protein binding to cyclin D1 (Toyoshima, H., and Hunter, T., Cell 78 (1994) 67-74). p27 is expressed periodically in proliferating cells. level are at a maximum during the G1 phase, decrease strongly as soon as the cells enter the S phase and remains at a low level until the cells reach the next G1 phase (Hengst, L., et al., Proc. Natl. Acad. Sci. USA 91 (1994) 5291-5295; Hengst, L., and Reed, S. I., Science 271 (1996) 1861-1864; Millard, S. S., et al., J. Biol. Chem. 272 (1997) 7093-7098). Moreover, p27 is induced by a broad range of antiproliferative signals in many different cell types (Hengst, L., and Reed, S. I., Curr. Top. Microbiol. Immunol. 227 (1998) 25-41). Thus, for example, p27 accumulates in cells which exit the cell cycle and become quiescent after withdrawing growth factors, or as a result of contact inhibition or removal of the substrate anchoring.

An abundance of experimental data indicates that the amount of p27 plays an important role in the regulation of the restriction point. For example most of the cyclin E/CDK2 and cyclin A/CDK2 complexes are inactive and if present associated with p27 in quiescent mouse fibroblasts. Hence inhibition of these complexes is due to p27 (Coats, S., et al., Science 272 (1996) 877-880). On the other hand the overexpression of p27 in cells frequently results in an arrest in the G1 phase (Polyak, K., et al., Cell 78 (1994) 59-66; Toyoshima, H., and Hunter, T., Cell 78 (1994) 67-74). Moreover, reducing the amount of p27 using antisense RNA technology prevents fibroblasts from becoming quiescent after serum withdrawal. These fibroblasts also have a shortened G1 phase (Coats, S., et al., Science 272 (1996) 877-880; Rivard, N., et al., J. Biol. Chem. 271 (1996) 18337-18341). This phenotype is otherwise observed when G1 cyclins are overexpressed and is therefore consistent with the CDK inhibitor function of p27 (Ohtsubo, M., et al., Mol. Cell. Biol. 15 (1995) 2612-2624; Quelle, D. E., et al., Genes Dev. 7 (1993) 1559-1571; Resnitzky, D., et al., Mol. Cell. Biol. 14 (1994) 1669-1679; Resnitzky, D., et al., Mol. Cell, Biol. 15 (1995) 4347-4352; Resnitzky, D., and Reed, S. I., Mol. Cell. Biol. 15 (1995) 3463-3469).

The role of p27 in cell cycle control has been confirmed by analysing p27-knockout mice. In two studies the p27 gene was completely deleted and in a third one it was replaced by a truncated p27 mutant lacking the CDK inhibitor domain (Fero, M. L., et al., Cell 85 (1996) 733-744; Nakayama, K, et al., Mol. Cell. Biol. 19 (1996) 1190-1201). All three mice strains have the same phenotype. It is mainly characterized by a gene dose-dependent increase in body size, general infertility of the female mice and deafness. The latter is caused by the continued proliferation of the hair cells of the corti organ in adult mice (Chen, P., and Segil, N., Development 126 (1999) 1581-1590; Lowenheim, H., et al, Proc. Natl. Acad Sci. USA 96 (1999) 4084-4088). Apparently the absence of p27 interferes with the ability of a number of cell types to withdraw from the cell cycle into the G0 phase or to differentiate during individual development (Vidal, A., and Koff, A., Gene 247 (2000) 1-15).

It is probable that p27 as a negative regulator of CDK activity plays a role as a tumour suppressor during the G1 phase. However, homozygotic inactivating mutations of the p27 gene are rarely found in human tumours (Kawamata, N., et al., Cancer Res. 55 (1995) 2266-2269; Morosetti, R., et al., Blood 86 (1995) 1924-1930; Pietenpol, J. A., et al., Cancer Res. 55 (1995) 1206-1210; Spirin, K. S., et al., Mol. Cell. 7 (1996) 639-650). Therefore p27 is not a tumour suppressor in the classical sense (Knudson, A. G., Jr., Proc. Natl. Acad. Sci. USA 68 (1971) 820-823). However, remarkably low amounts of p27 are frequently detected in human tumours and these low amounts of p27 frequently correlate with high tumour aggressiveness and high patient mortality (Slingerland, J., and Pagano, M., J. Cell. Physiol. 183 (2000) 10-17). A general increase in the incidence of tumours is not observed in unchallenged p27-negative mice; however, the animals suffer from a change in the pituitary which has been classified as a benign adenoma (Nakayama, K., et al., Mol. Cell. Biol. 19 (1996) 1190-1201). Moreover p27-negative as well as p27-heterozygotic mice have an increased rate of tumours when irradiated or when treated with chemical carcinogens compared to the control group. This indicates a pivotal role of the amount of p27 in preventing tumours. p27 has been referred to as a “haplo-insufficient tumour suppressor”, since no LOH has been found in tumours (Fero, M. L., et al., Nature 396 (1998) 177-180).

The activity and amount of the inhibitor is of major importance for the function of p27. Expression of p27 can be regulated at various levels. In many cases it is not transcriptional but involves regulation of translation or stability of the protein (Hengst, L., and Reed, S. I., Curr. Top. Microbiol. Immunol. 227 (1998) 25-41).

p27-mRNA translation increases when normal diploid fibroblasts (HS68) exit the cell cycle due to contact inhibition. During the cell cycle, translation of p27-mRNA is subject to periodic oscillations. It is increased in HeLa cells arrested in G1 phase by lovastatin, compared to S phase arrested cells (Hengst, L., and Reed, S. I., Science 271 (1996) 1861-1864). A second mechanism of modulating the amount of a protein is to regulate its stability. p27 is degraded through the ubiquitin-proteasome pathway (Pagano, M., et al., Science 269 (1995) 682-685). The ubiquitination of p27 occurs at the G1/S phase transition by the SCF-Skp2 ubiquitin ligase complex and requires Cks1 as a cofactor (Carrano, A. C., et al., Nat Cell. Biol. 1 (1999) 193-199; Morimoto, M., et al., Biochem. Biophys. Res. Commun. 270 (2000) 1093-1096; O'Hagan, R. C., et al., Genes Dev. 14 (2000) 2185-2191; Spruck, C., et al, Mol. Cell 7 (2001) 639-650; Sutterluty, H., et al., Nat. Cell. Biol. 1 (1999) 207-214; Tsvetkov, L. M., et al., Curr. Biol. 9 (1999) 661-664). p27 is more efficiently ubiquitinated and degraded in extracts of proliferating cells and cells in the S phase than in extracts of quiescent cells and cells in the G1 phase (Brandeis, M., and Hunt, T., Embo J. 15 (1996) 5280-5289; Montagnoli, A., et al., Genes Dev. 13 (1999) 1181-1189; Nguyen, H., et al., Mol. Cell. Biol. 19 (1999) 1190-1201; Pagano, M., et al., Science 269 (1995) 682-685); the half live of the protein changes from 2,5 hours in G1 phase to less then 20 minutes in S phase (Hengst, L., and Reed, S. I., Science 271 (1996) 1861-1864).

In late G1, S and G2 phase p27 must be phosphorylated at the threonine residue 187 by CDK2 in order to become a substrate of the SCF-SKp2 complex and to be subsequently degraded by the proteasome during the (Malek, N. P., et al., Nature 413 (2001) 323-327; Montagnoli, A., et al., Genes Dev. 13 (1999) 1181-1189; Muller, D., et al., Oncogene 15 (1997) 2561-2576; Nguyen, H., et al., Mol. Cell. Biol. 19 (1999) 1190-1201; Sheaff, R. J., et al., Genes Dev. 11 (1997) 1464-1478; Vlach, J., et al., Embo J. 16 (1997) 5334-5344). During G1 phase p27 is degraded by a second degradation pathway which is also ubiquitin-dependent but independent of the phosphorylation of threonine 187 (Malek, N. P., et al., Nature 413 (2001) 323-327; Hara, T., et al., J. Biol. Chem. 276 (2001) 48937-48943). In this case the ubiquitination of p27 appears to take place in the cytoplasm.

In addition to their role as inhibitors for most cyclin/CDK complexes, Cheng et al., 1999 report that the p21^{Cip1} and p27^{Kip1} inhibitors are essential activators of cyclin D-dependent kinases in murine fibroblasts. They find that the activation of cyclin D-CDK4 in mitogen-stimulated fibroblasts depends redundantly on the presence of p21^{Cip1} and p27^{Kip1}. The conclusions of this study are primarily based on the analysis of primary mouse embryonic fibroblasts lacking p21^{Cip1} and p27^{Kip1}, although in some experiments these cells are transduced by p21^{Cip1} and p27^{Kip1} encoding retroviruses. However, p21^{Cip1} and p27^{Kip1} proteins are never investigated or detected as such or especially modifications of the proteins have not been analysed or investigated. The conclusion that p21 or p27 act as constitutive activators of CDK complexes has since been called into question by others (see below, Bagui et al., (2000). MCB 20, 8748-8757; Bagui et al., (2003). MCB 23, 7285-7290).

LaBaer et al. (Genes & Development 11 (1999) 847-862) disclose the promotion association of cdk4 with D-type cyclins by p21^{Cip1}, p27^{Kip1} and p57^{Kip2}. Both in vivo and in vitro, the abundance of assembled cdk4/cydin D complex increases directly with increasing inhibitor levels. The complexes and the components thereof are analysed by Western Blots after immunoprecipitations. LaBaer et al. find that low concentrations of p21 stimulate cyclin D/CDK complex assembly and kinase activity, whereas high concentrations of p21 inhibit this kinase activity.

A number of alternative explanations are offered for the observed results. The majority of proposed mechanisms suggests that lack of cyclin D/CDK inhibition of p21^{Cip1} or p27^{Kip1} is an intrinsic property of these inhibitors and does not require any modification of the inhibitor molecule. This model suggests that the stoichiometry of the Cip/Kip protein within cyclin D/CDK complexes determines whether it acts as an inhibitor or an activator of this complex. This mechanism has originally been uncovered by Shang and coworkers who reported that more then one inhibitor molecule per CDK complex is required for CDK inactivation (Zhang et al., Genes & Development 8 (1994). 1750-1774)). Later it was suggested that this model is accurate only for cyclin D/CDK complexes (Blain et al., JBC 272 (1997), 25863-25872). According to this model, lack of inhibition is an intrinsic property of the unmodified p21^{Cip1} or p27^{Kip1} molecules and restricted to cyclin D/CDK complexes. Therefore this mechanism does not involve any modification of the Cip/Kip protein. This model is also favoured in the study by LaBaer et al. as they find that the role of p21 as an activator or inhibitor of CDKs depends directly on its expression level. The second group of possible mechanisms involves the absence of cyclin D/CDK4 associated proteins like Hsc70 (as reported by Diehl et al., MCB 23 (2003), 1764-1774). The publication by Cheng et al., (EMBO J. 18 (1999) discusses these models and, among various alternative mechanisms, also modifications on CDKs, cyclins, interacting proteins or CDK inhibitor proteins. As one possible modification it is suggested that phosphorylation events may occur either on the CDK inhibitor, the cyclin/CDK complex or on other undefined interacting proteins that could play a role in assembly.

The observations by Cheng et al. and LaBaer et al. are still controversal in the field as reviewed for example by Olashaw et al., (2004). Cell Cycle 3, 263-264. There are proposals that Cip/Kip proteins are always and unconditionally inhibitors of cyclin D/CDK complexes (Bagui et al., (2000). MCB 20, 8748-8757; Bagui et al., (2003). MCB 23, 7285-7290) contradicting the observation by Cheng et al., by using identical experimental systems and approaches.

### SUMMARY OF THE INVENTION

There is still a need to identify markers that can be used to analyse whether patients afflicted with cancer have a high risk for tumour progression. The tyrosine modification of p27 disclosed herein should allow to determine whether p27 acts as a CDK inhibitor or activator, and thus be an excellent marker for tumour progression. Numerous phosphorylations have been investigated in most detail for Cip/Kip proteins including p27. It is important to note that up to now only serine and threonine phosphorylations of Cip/Kip proteins including p27 have been reported on various sites. Tyrosine phosphorylation of p27 and other Cip/Kip proteins has never been reported and even been excluded in some studies (for example: Ishida et al., JBC 275 (2000) 25146-25154, Sheaff, et al., Genes & Development 11 (1997), 1464-1478. Muller et al. Oncogene 15 (1997), 2561-2576). Phosphorylation of Cip/Kip proteins on any tyrosine residue has therefore never been reported and is an entirely novel modification.

The embodiments of the present invention are based on the finding that the CDK inhibitor proteins like p27Kip1 become phosphorylated on tyrosine residue 88 (or the homologous conserved residue in p21 and p57) and tyrosine residue 89 by tyrosine directed protein kinases. This modification has three potential consequences:
The inhibitory activity of p27 is impaired.
The inhibitor becomes a better substrate for CDK2-dependent phosphorylation that triggers its SCF-Skp2-dependent degradation.
The modified inhibitor acts as an activator of cyclin-dependent kinases by exerting CDK/cyclin assembly factor activity.
The eucaryotic cell cycle is regulated by the oscillating activity of various cyclin-dependent kinases (CDKs). CDK kinase activity is regulated by CDK inhibitor proteins. The amount of the CDK inhibitor protein p27^{Kip1} plays a key role in the transition of the cell from the G1 to the S phase. p27^{Kip1} increases during the G0 or the G1 phase and decreases rapidly at the onset of the S phase. Binding of p27^{Kip1} to the CDK2 kinase complexes in the G1 phase inactivates them and can thus prevent transition into S phase. A reduced amount of p27^{Kip1} at the G1/S phase transition is frequently found in various tumour tissues. The smaller level of the inhibitor is associated with a high patient mortality and an aggressive course of the disease. The SH3 domain protein Grb2 was identified as interaction partner of the inhibitor p27^{Kip1}.This interaction involves the C-terminal SH3 domain of Grb2 and a proline-rich region in p27. In search for other SH3 domains that are able to associate with the proline-rich domain in p27, it was discovered the src-related tyrosine linase Lyn as a binding partner. Using recombinant tyrosine kinases, it was discovered that p27 is a substrate for tyrosine linases including scr, Abl, Bcr-Abl or Lyn. Some tyrosine kinases may interact with p27 by using their SH3 domains or the adapter function of Grb2, however SH3-independent mechanisms may direct tyrosine kinases to p27. These may not involve a direct interaction between the inhibitor and the kinase. It was demonstrated initially by phosphoamino acid analysis and later using phospho-specific antibodies that p27 and p21 become phosphorylated on tyrosine in vivo. Mutational analysis and later generation of phospo-specific antibodies allowed to determine that preferably tyrosine residue 88 but also tyrosine 89 are modified by Bcr-Abl in vitro and in vivo. Tyrosine residue 88 of p27 is a substrate for Abl, Bcr-Abl, Lyn and src kinases.

According to crystal structural data of the trimeric complex consisting of p27^{Kip1}, CDK2 and cyclin A, the tyrosine residue 88 of p27^{Kip1} integrates into the ATP binding pocket of the kinase and blocks it for ATP binding. Hence it was examined to what extent a phosphorylation of p27^{Kip1} on tyrosine 88 or 89 influences the activity of the inhibitor. The tyrosine phosphorylation of p27^{Kip1} does not prevent binding to the CDK complex. However, using in vitro-phosphorylated p27^{Kip1} it was shown that a tyrosine phosphorylation leads to a reduction of the activity of the inhibitor. Especially tyrosine 88 phosphorylation impairs CDK inhibition by p27.

The negative charge of the phosphate group on tyrosine 88 may interfere with the positioning of the tyrosine residue in the purine binding pocket of the CDK. This allows the inhibitor to promote activation of the kinase by promoting CDK/cyclin D assembly, converting the inhibitor into an activator of cyclin-dependent kinases. If one assumes that the inhibitor binds to CDK4/cyclin D in a similar way as to cyclin A/CDK2 in the X-ray structure, one could speculate that this reduction may explain the contradictory observations of the inhibition of cyclinD/CDK4 inhibition by p21 or p27 proteins: After phosphorylation of the tyrosine 88 residue, the ability of p27 or p21 to inhibit CDKs is substantially reduced. By binding to both kinase subunits, CDK4,6 and D-type cyclins modified p27 may stimulate complex assembly of this kinase.

It is interesting that the tyrosine phosphorylation of the inhibitor significantly increases the phosphorylation of p27^{Kip1} at threonine 187 by the bound CDK complex. The phosphorylation of p27^{Kip1} at threonine 187 is an initial signal to degrade p27^{Kip1} by the 26S proteosome. Threonine 187-phosphorylated p27^{Kip1} is recognized by the E3-ligase complex SCF-Skp2/Cks1 and ubiquitinated. Therefore tyrosine phosphorylation of p27 may prime the inhibitor for the ubiquitin-dependent degradation via the SCF-Skp2-Cks1/proteasome pathway.

Consistent with its central role in growth control, it was found that p27 tyrosine phosphorylation is enhanced in cells transfected with Bcr-Abl, src or Lyn tyrosine kinases. In Bcr-Abl positive leukaemia cell lines enhanced phosphorylation of endogenous p27 was detected and this phosphorylation was lost upon inhibition of Bcr-Abl by imatinib-mesylate. Increased tyrosine phosphorylated p27 should therefore be a valuable marker in various tumours. The detection of tyrosine-phosphorylated p27 or the ratio of tyrosine-phosphorylated versus non-modified p27 could be used as a prognostic or monitoring marker in tumors. A mutant p27was generated where tyrosine 88 has been substituted by phenylalanine. The mutant protein still acts as a CDK inhibitor. This mutant protein can be used as a modification-resistant CDK inhibitor that cannot be inactivated or converted into an activator by tyrosine kinases. If used in therapy, the CDK inhibitory domain of p27 will be significantly improved over the wild-type CDK inhibitory domain. Expression of the mutant protein p27-Y88F mutant in human Bcr-Abl transformed leukemia cells (K562) confirmed that this protein is more stable than p27. Importantly, whereas p27 that can be phosphorylated on tyrosine 88 failed to inhibit cell cycle progression and the G1/S transition of these CML cells, the same moderate expression of the phenylalanine mutant in position 88 did inhibit the progression into S-phase in these leukaemia cells. These experiments suggest that mutant p27 may be a potent growth inhibitor in certain tumor cells. Since the tyrosine residue 88 of p27 is highly conserved in the related inhibitors p21 and p57, these inhibitors can also be regulated by tyrosine phosphorylation.

Therefore, in an embodiment of the invention, a polypeptide is provided comprising the amino acid sequence SEQ ID NO: 2, 4 or 6 whereby in the polypeptide comprising the amino acid sequence SEQ ID NO: 2, the tyrosine residue at position 88 and/or the tyrosine residue at position 89 in SEQ ID NO: 2 is/are phosphorylated, or whereby in the polypeptide comprising the amino acid sequence SEQ ID NO: 4, the tyrosine residue at position 77 in SEQ ID NO 4 is phosphorylated, or whereby in the polypeptide comprising the amino acid sequence SEQ ID NO: 6, the tyrosine residue at position 91 in SEQ ID NO 6 is phosphorylated.

In another embodiment of the invention, a peptide fragment is provided with a minimum length of 6 amino acids of a polypeptide comprising the amino acid sequence SEQ ID NO: 2, 4 or 6, whereby in the peptide fragment of a polypeptide comprising the amino acid sequence SEQ ID NO: 2, the tyrosine residue at position 88 and/or the tyrosine residue at position 89 in SEQ ID NO: 2 is/are phosphorylated, or whereby in the peptide fragment of a polypeptide comprising the amino acid sequence SEQ ID NO: 4, the tyrosine residue at position 77 in SEQ ID NO 4 is phosphorylated, or whereby in the peptide fragment of a polypeptide comprising the amino acid sequence SEQ ID NO: 6, the tyrosine residue at position 91 in SEQ ID NO 6 is phosphorylated.

In another embodiment of the invention, a non-(phosphorylable)phosphorylatable polypeptide is provided comprising the amino acid sequence SEQ ID NO: 2, 4 or 6 or comprising the amino acid residues 1 to 95 of SEQ ID NO: 2 or comprising the amino acid residues 1 to 85 of SEQ ID NO: 4 or comprising the amino acid residues 1 to 100 of SEQ ID NO: 6 or comprising the amino acid residues 50 to 95 of SEQ ID NO: 2 or comprising the amino acid residues 38 to 85 of SEQ ID NO: 4 or comprising the amino acid residues 50 to 100 of SEQ ID NO: 6 characterized in that in the polypeptide derived from the polypeptide with the amino acid sequence SEQ ID NO: 2, the amino acid residue at position 88 and/or position 89 in SEQ ID NO: 2, or in the polypeptide derived from the polypeptide with the amino acid sequence SEQ ID NO: 4, the amino acid residue at position 77 in SEQ ID NO: 4 or in the polypeptide derived from the polypeptide with the amino acid sequence SEQ ID NO: 6, the amino acid residue at position 91 in SEQ ID NO: 6 is a non-phosphorylatable amino acid residue, preferably a phenylalanine residue.

In another embodiment of the invention, a non-phosphorylatable peptide fragment is provided with a minimum length of 6 amino acids of a polypeptide comprising the amino acid sequence SEQ ID NO: 2, 4 or 6, characterized in that in the peptide fragment of a polypeptide comprising the amino acid sequence SEQ ID NO: 2, the peptide fragment comprises at least one of the flanking amino acid residues of the amino acid residue at position 88 or 89 in SEQ ID NO: 2 and the residue at position 88 and/or the residue at position 89 in SEQ ID NO: 2 is a non-phosphorylable amino acid residue, preferably a phenylalanine residue, or in the peptide fragment of a polypeptide comprising the amino acid sequence SEQ ID NO: 4, the peptide fragment comprises at least one of the flanking amino acid residues of the amino acid residue at position 77 in SEQ ID NO: 4 and the residue at position 77 in SEQ ID NO: 4 is a non-phosphorylable amino acid residue, preferably a phenylalanine residue, or in the peptide fragment of a polypeptide comprising the amino acid sequence SEQ ID NO: 6, the peptide fragment comprises at least one of the flanking amino acid residues of the amino acid residue at position 91 in SEQ ID NO: 6 and the residue at position 91 in SEQ ID NO: 6 is a non-phosphorylable amino acid residue, preferably a phenylalanine residue.

In yet another embodiment of the invention, a nucleic acid molecule is provided encoding a polypeptide according to the invention or a peptide fragment according to the invention or a DNA vector comprising a nucleic acid molecule encoding a polypeptide according to the invention or a peptide fragment according to the invention.

In still another embodiment a variant or peptidomimetics of the polypeptide, peptide fragment or non-phosphorylable peptide fragment is provided. In still another embodiment, an isolated antibody is provided which specifically binds to a polypeptide according to the invention or to a peptide fragment according to the invention and which has less than 10% cross reactivity with the non-phosphorylated polypeptide or non-phosphorylated peptide fragment.

In another embodiment of the invention, the hybridoma cell lies Mab<p27kip1>15 and Mab<p27kip1>388 are provided that were deposited on Oct. 6, 2004 with the Deutsche Sammlung für Mikroorganismen und Zellkulturen GmbH, Braunschweig, Germany (German Collection of Microorganisms and Cell Cultures).

In an embodiment of the invention, a polypeptide according to the invention, a peptide fragment according to the invention, a variant or peptidomimetics according to the invention, a nucleic acid molecule according to the invention or an antibody according to the invention is provided for use in medicine or may be contained in a pharmaceutical composition. The said substances according to the invention may also be used for the preparation of a pharmaceutical composition for the treatment of hyperproliferative disease, preferably cancer.

In still another embodiment of the invention, a method is provided for the determination of the amount or presence of a polypeptide according to the invention or a peptide fragment according to the invention in a sample comprising the steps of
a) providing a sample suspected to contain the polypeptide or the peptide fragment,
b) incubating the sample in the presence of an antibody according to the invention, and
c) determining the binding product between the polypeptide and the antibody thereby concluding that the polypeptide or peptide fragment is present or thereby deriving the amount of the polypeptide or the peptide fragment.

In another embodiment of the invention, a method of selecting a composition for inhibiting the progression of cancer in a patient is provided, the method comprising providing a sample comprising cancer cells from the patient, separately exposing aliquots of the sample in the presence of a plurality of test compositions, comparing the level of expression of the polypeptide according to the invention or a peptide fragment according to the invention in each of the aliquots; and selecting one of the test compositions which alters the level of expression of the polypeptide in the aliquot containing that test composition, relative to other test compositions.

In another embodiment of the invention, a method to predict which patients will respond to a drug, preferentially a tyrosine kinase inhibitor drug in patients with a disorder whose underlying pathology involves the discontrol of a tyrosine linase is provided comprising contacting a sample from a patient with an antibody according to the invention, determining the level of phosphorylated polypeptide bound by the antibody of the first step, comparing the level of phosphorylated polypeptide determined in the second step for the sample with the level of phosphorylated protein in a reference sample, thereby detecting the responsiveness to a tyrosine kinase inhibitor drug in patients with a disorder whose underlying pathology involves the discontrol of a tyrosine kinase.

In comparison to the normal level of the polypeptide or peptide fragment according to the invention and further variations, this method may also be used for determining whether or not a human cancer cell containing patient sample has potential for tumor progression or for the prediction which patients with a disorder whose underlying pathology involves the discontrol of a tyrosine kinase will respond to a drug, preferentially a tyrosine kinase inhibitor drug, selecting a composition for inhibiting the progression of cancer in a patient and deriving a candidate agent. Further, the polypeptide according to the invention or a peptide fragment according to the invention may be used as an immunogen to generate or produce antibodies, in particular monoclonal antibodies. In another embodiment of the invention, the antibody according to the invention is used for the determination of the amount or the presence of a polypeptide or peptide fragment according to the invention in a sample.

In yet another embodiment of the invention, a kit for the detection or determination of the amount of the polypeptide according to the invention or the peptide fragment according to the invention in a biological sample is provided which comprises:
(a) an antibody according to the invention and
(b) a label for qualitatively or quantitatively detecting an immunoconjugate of the antibody and the polypeptide or the peptide fragment.

In still another embodiment of the invention, a virus particle comprising a nucleic acid molecule or a vector according to the invention is provided and a mammalian cell comprising a nucleic acid molecule or a vector according to the invention. In another embodiment of the invention, a virus particle or a mammalian cell for use in medicine is provided, a pharmaceutical composition comprising a virus particle according to the invention or a mammalian cell according to the invention and a pharmaceutically acceptable carrier. In another embodiment of the invention, a virus particle according to the invention or a mammalian cell according to the invention is used for the preparation of a pharmaceutical composition for the treatment of cancer.

### DETAILED DESCRIPTION OF THE INVENTION

The one letter code for amino acids is used often throughout this application which can be found in standard textbooks. The most important abbreviations for this application is Y for TYR or tyrosine and F for PHE or phenylalanine. The abbreviations are A, Ala: Alanine, R, Arg: Arginine, N,Asn: Asparagine, D,Asp: Aspartic acid, C,Cys: Cysteine, Q, Gln: Glutamine, E, Glu: Glutamic acid, G, Gly: Glycine, H, His: Histidine, I, Ile: Isoleucine, L, Leu: Leucine, K, Lys: Lysine, M, Met: Methionine, F, Phe: Phenylalanine, P, Pro: Proline, S, Ser: Serine, T, Thr: Threonine, W, Trp: Tryptophan, Y, Tyr: Tyrosine, V, Val: Valine.

### I. Isolated Proteins and Antibodies

The invention provides a recombinant polypeptide or protein with an amino acid sequence according to SEQ ID NO: 2, 4 or 6 and which can be encoded by the DNA sequence shown in SEQ ID NO: 1, 3 or 5 and which is phosphorylated at the tyrosine residues 88 and/or 89 or at the positions in p21 and p57 that correspond to tyrosine residue 88 in p27 that can be identified by sequence alignment (see FIG. 1). The sequence alignment in FIG. 1 also allows identifying other corresponding amino acids or functional elements of the polypeptides.

In an embodiment of the invention, phosphorylated forms of p21, p27 and p57 are contemplated, i.e. a polypeptide is provided comprising the amino acid sequence SEQ ID NO: 2, 4 or 6 whereby in the polypeptide comprising the amino acid sequence SEQ ID NO: 2, the tyrosine residue at position 88 and/or the tyrosine residue at position 89 in SEQ ID NO: 2 is/are phosphorylated, or whereby in the polypeptide comprising the amino acid sequence SEQ ID NO: 4, the tyrosine residue at position 77 in SEQ ID NO 4 is phosphorylated, or whereby in the polypeptide comprising the amino acid sequence SEQ ID NO: 6, the tyrosine residue at position 91 in SEQ ID NO 6 is phosphorylated. An embodiment of the invention specifically contemplates fusion proteins with other partners. Preferably, the polypeptide consists of the amino acid sequence SEQ ID NO: 2, 4 or 6 whereby the said tyrosines are phosphorylated. The term “are/is phosphorylated” means in other words in the context of the invention that if the tyrosine is phosphorylated, it is a phosphotyrosine residue or a phosphodiester between H₃PO₄ and the (substituted) phenol moiety of the tyrosine. Preferred in all above-mentioned embodiments is SEQ ID NO: 2.

In another embodiment of the invention, even further phosphorylated forms of p27 are provided, i.e. a polypeptide comprising the amino acid sequence SEQ ID NO: 2, whereby the tyrosine residues at position 88 and/or the tyrosine residue at position 89 in SEQ ID NO: 2 are phosphorylated and whereby the serine residue at position 10 and/or 12 and/or the threonine residue at position 157 and/or the threonine residue at position 187 and/or the threonine residue in position 198 in SEQ ID NO: 2 are phosphorylated. It is preferred that only position 187 is phosphorylated. An embodiment of the invention specifically contemplates fusion proteins with other partners. Preferably, the polypeptide consists of the amino acid sequence SEQ ID NO: 2 whereby at least one of the said tyrosines is phosphorylated.

In another embodiment of the invention, a peptide fragment is provided with a minimum length of 6 amino acids of a polypeptide comprising the amino acid sequence SEQ ID NO: 2, 4 or 6, whereby in the peptide fragment of a polypeptide comprising the amino acid sequence SEQ ID NO: 2, the tyrosine residue at position 88 and/or the tyrosine residue at position 89 in SEQ ID NO: 2 is/are phosphorylated, or whereby in the peptide fragment of a polypeptide comprising the amino acid sequence SEQ ID NO: 4, the tyrosine residue at position 77 in SEQ ID NO 4 is phosphorylated, or whereby in the peptide fragment of a polypeptide comprising the amino acid sequence SEQ ID NO: 6, the tyrosine residue at position 91 in SEQ ID NO 6 is phosphorylated. In another embodiment of the invention, a peptide fragment is provided with a minimum length of 6 amino acids that is a peptide fragment of a polypeptide comprising the amino acid sequence SEQ ID NO: 2, 4 or 6, whereby the peptide fragment comprises the tyrosine residue at position 88 and/or the tyrosine residue (located) at position 89 in SEQ ID NO: 2 or whereby the peptide fragment comprises the tyrosine residue (located) at position 77 in SEQ ID NO 4 or whereby the peptide fragment comprises the tyrosine residue (located) at position 91 in SEQ ID NO 6

characterized in that
the tyrosine residue (corresponding to the tyrosine residue (located)) at position 88 and/or the tyrosine residue (corresponding to the tyrosine residue (located)) at position 89 in SEQ ID NO: 2 is/are phosphorylated in the peptide fragment of a polypeptide comprising the amino acid sequence SEQ ID NO: 2, or
the tyrosine residue (corresponding to the tyrosine residue (located)) at position 77 in SEQ ID NO 4 is phosphorylated in the peptide fragment of a polypeptide comprising the amino acid sequence SEQ ID NO: 4, or
the tyrosine residue (corresponding to the tyrosine residue (located)) at position 91 in SEQ ID NO 6 is phosphorylated in the peptide fragment of a polypeptide comprising the amino acid sequence SEQ ID NO: 6.

Preferred in the above-mentioned embodiments is SEQ ID NO: 2 whereby preferably the tyrosine residue (corresponding to the tyrosine residue (located)) at position 88 in SEQ ID NO: 2 is phosphorylated in the peptide fragment of a polypeptide comprising the amino acid sequence SEQ ID NO: 2.

The length of 6 amino acids corresponds to an epitope. In a more preferred embodiment of the invention, the peptide fragment is provided with a minimum length of 8, more preferably 10 or 12 amino acids in particular where the phosphorylated tyrosines are located in the middle of the peptide fragment. Preferably, the peptide fragment has a minimum length of 15 amino acids. Overall the peptide fragment may be up to 30 amino acids long. An embodiment of the invention specifically contemplates fusion peptides with other partners. Preferably, the peptide fragments only consists of fragments of the amino acid sequences SEQ ID NO: 2, 4 or 6 whereby at least one of the said tyrosines is phosphorylated.

Preferred peptide fragments of p27 have the amino acid sequence EKGSLPEFYYRPPRP (15 amino acids (AA) (SEQ ID NO: 21)), SLPEFYYRPPRP (12 amino acids (AA) (SEQ ID NO: 22)), LPEFYYRPPR (10 amino acids (AA) (SEQ ID NO: 23), PEFYYRPP (8 amino acids (AA) (SEQ ID NO: 24)) or EFYYRP (6 amino acids (AA) (SEQ ID NO: 25)) whereby at least one of the tyrosines is phosphorylated. Preferred p21 and p57 peptide fragments can be identified accordingly by identifying the corresponding amino acid residues from the sequence alignment in FIG. 1.

The peptide fragments are particularly useful to be used as an antigen for producing antibodies, particularly monoclonal antibodies.

The invention also discloses polypeptides, fragments thereof as the CDK inhibitory or binding domains or peptide fragments where the said tyrosine is preferably substituted by phenylalanine or any other amino acid to make the protein or polypeptide resistant to tyrosine modification at the described positions, i.e. phosphorylation, whereby in p27 only one tyrosine may be exchanged making it resistant to phosphorylation by protein kinases only phosphorylating at the indicated position. Therefore, the amino acid residue tyrosine should be absent, preferably also the amino acids serine, threonine or the amino acids with carboxylate carrying side chains as aspartate and glutamate which mimick a phosphate group. The preferred exchange is phenylalanine but is evident for an expert skilled in the art that other amino acid residues may be incorporated as well.

Therefore, in a preferred embodiment of the invention, a non-phosphorylable polypeptide is provided comprising the amino acid sequence SEQ ID NO: 2, 4 or 6 or comprising the amino acid residues 1 to 95 of SEQ ID NO: 2 or comprising the amino acid residues 1 to 85 of SEQ ID NO: 4 or comprising the amino acid residues 1 to 100 of SEQ ID NO: 6 or comprising the amino acid residues 50 to 95 of SEQ ID NO: 2 or comprising the amino acid residues 38 to 85 of SEQ ID NO: 4 or comprising the amino acid residues 50 to 100 of SEQ ID NO: 6 characterized in that in the polypeptide derived from the polypeptide with the amino acid sequence SEQ ID NO: 2, the amino acid residue at position 88 and/or position 89 in SEQ ID NO: 2, or in the polypeptide derived from the polypeptide with the amino acid sequence SEQ ID NO: 4, the amino acid residue at position 77 in SEQ ID NO: 4 or in the polypeptide derived from the polypeptide with the amino acid sequence SEQ ID NO: 6, the amino acid residue at position 91 in SEQ ID NO: 6 is a non-phosphorylable amino acid residue, preferably a phenylalanine residue.

In another preferred embodiment of the invention, a non-phosphorylable polypeptide is provided comprising the amino acid sequence SEQ ID NO: 2, 4 or 6 or comprising the amino acid residues 1 to 95 of SEQ ID NO: 2 or comprising the amino acid residues 1 to 85 of SEQ ID NO: 4 or comprising the amino acid residues 1 to 100 of SEQ ID NO: 6 or comprising the amino acid residues 50 to 95 of SEQ ID NO: 2 or comprising the amino acid residues 38 to 85 of SEQ ID NO: 4 or comprising the amino acid residues 50 to 100 of SEQ ID NO: 6

characterized in that
the amino acid residue in the non-phosphorylable polypeptide derived from the polypeptide with the amino acid sequence SEQ ID NO: 2, that is (located) at position 88 and/or position 89 in SEQ ID NO: 2 (and is located in the corresponding position in the non-phosphorylable polypeptide) or
the amino acid residue in the non-phosphorylable polypeptide derived from the polypeptide with the amino acid sequence SEQ ID NO: 4 that is (located) at position 77 in SEQ ID NO: 4 (and is located in the corresponding position in the non-phosphorylable polypeptide) or
the amino acid residue in the non-phosphorylable polypeptide derived from the polypeptide with the amino acid sequence SEQ ID NO: 6 that is (located) at position 91 in SEQ ID NO: 6 (and is located in the corresponding position in the non-phosphorylable polypeptide)
is a non-phosphorylable amino acid residue, preferably a phenylalanine residue.

An embodiment of the invention specifically contemplates fusion proteins with other partners. Preferably, the polypeptide consists of the amino acid sequence SEQ ID NO: 2, 4 or 6 whereby at least one of the said tyrosines is phosphorylated.

In another preferred embodiment of the invention, a peptide fragment is provided with a minimum length of 6 amino acids of a polypeptide comprising the amino acid sequence SEQ ID NO: 2, 4 or 6, characterized in that in the peptide fragment of a polypeptide comprising the amino acid sequence SEQ ID NO: 2, the peptide fragment comprises at least one of the flanking amino acid residues of the amino acid residue at position 88 or 89 in SEQ ID NO: 2 and the (amino acid) residue at position 88 and/or the (amino acid) residue at position 89 in SEQ ID NO: 2 is a non-phosphorylable amino acid residue, preferably a phenylalanine residue, or in the peptide fragment of a polypeptide comprising the amino acid sequence SEQ ID NO: 4, the peptide fragment comprises at least one of the flanking amino acid residues of the amino acid residue at position 77 in SEQ ID NO: 4 and the (amino acid) residue at position 77 in SEQ ID NO: 4 is a non-phosphorylable amino acid residue, preferably a phenylalanine residue, or in the peptide fragment of a polypeptide comprising the amino acid sequence SEQ ID NO: 6, the peptide fragment comprises at least one of the flanking amino acid residues of the amino acid residue at position 91 in SEQ ID NO: 6 and the (amino acid) residue at position 91 in SEQ ID NO: 6 is a non-phosphorylable amino acid residue, preferably a phenylalanine residue.

In another preferred embodiment of the invention, a peptide fragment is provided with a minimum length of 6 amino acids of a polypeptide comprising the amino acid sequence SEQ ID NO: 2, 4 or 6,

characterized in that

the peptide fragment of a polypeptide comprising the amino acid sequence SEQ ID NO: 2 comprises at least one of the flanking amino acid residues of the amino acid residue (corresponding to the amino acid residue (located)) at position 88 or 89 in SEQ ID NO: 2 and the amino acid residue (corresponding to the amino acid residue (located)) at position 88 and/or the amino acid residue (corresponding to the amino acid residue (located)) at position 89 in SEQ ID NO: 2 is a non-phosphorylable amino acid residue, preferably a phenylalanine residue,

or the peptide fragment of a polypeptide comprising the amino acid sequence SEQ ID NO: 4, comprises at least one of the flanking amino acid residues of the amino acid residue (corresponding to the amino acid residue (located)) at position 77 in SEQ ID NO: 4 and the amino acid residue (corresponding to the amino acid residue (located)) at position 77 in SEQ ID NO: 4 is a non-phosphorylable amino acid residue, preferably a phenylalanine residue,
or the peptide fragment of a polypeptide comprising the amino acid sequence SEQ ID NO: 6, comprises at least one of the flanking amino acid residues of the amino acid residue (corresponding to the amino acid residue (located)) at position 91 in SEQ ID NO: 6 and the amino acid residue (corresponding to the amino acid residue (located)) at position 91 in SEQ ID NO: 6 is a non-phosphorylable amino acid residue, preferably a phenylalanine residue.

In a more preferred embodiment of the invention, the peptide fragment is provided with a minimum length of 8, more preferably 10 or 12 amino acids in particular where the residues exchanged for the phosphorylatable tyrosines are located in the middle of the peptide fragment. Preferably, the peptide fragment has a minimum length of 15 amino acids. Overall the peptide fragment may be up to 30 amino acids long. An embodiment of the invention specifically contemplates fusion peptides with other partners. Preferably, the peptide fragments only consists of fragments of the amino acid sequences SEQ ID NO: 2, 4 or 6 whereby at least one of the said tyrosines are exchanged for a non-phosphorylable amino acids, preferably phenylalanin.

Preferred non-phosphorylable peptide fragments of p27 have the amino acid sequence EKGSLPEF(Y/F)(Y/F)RPPRP (15 amino acids (AA) (derived from SEQ ID NO: 21)), SLPEF(Y/F)(Y/F)RPPRP (12 amino acids (AA) (derived from SEQ ID NO: 22)), LPEF(Y/F)(Y/F)RPPR (10 amino acids (AA) (derived from SEQ ID NO: 23)), PEF(Y/F)(Y/F)RPP (8 amino acids (AA) (derived from SEQ ID NO: 24)) or EF(Y/F)(Y/F)RP (6 amino acids (AA) (derived from SEQ ID NO: 25)) whereby at least one of the tyrosines is substituted by phenylalanine as denoted by (Y/F). Preferred p21 and p57 peptide fragments can be identified accordingly by identifying the corresponding amino acid residues from the sequence alignment in FIG. 1.

In still another embodiment a variant or peptidomimetics of the polypeptide, peptide fragment or non-phosphorylable peptide fragment is provided.

In all polypeptides or peptide fragments according to the invention SEQ ID NO: 2 is preferred. The invention further contemplates a method for producing a polypeptide or peptide fragment according to the invention by expressing an exogenous DNA in prokaryotic or eukaryotic host cells and isolation of the desired protein, wherein the protein is preferably coded by the nucleic acid sequence as shown in SEQ ID NO:1. The protein can be isolated from the cells or the culture supernatant and purified by chromatographic means, preferably by ion exchange chromatography, affinity chromatography and/or reverse phase HPLC. The isolated protein is then phosphorylated using the described, isolated and commercially available protein kinases including the src family kinases, Abl kinases, receptor-associated kinases. In more detail in order to generate p27 protein phosphorylated on tyrosyl side chains the p27 cDNA is expressed in E. coli by cloning the cDNA into an eucaryotic expression vector, e.g. pET8c. Expression of p27 is induced with IPTG at 37° C. for up to 4 hours. Recombinant p27 protein is purified by heat treatment, anion and cation exchange chromatography and gel filtration as described in methods 3.4.8 to 3.4.11. Tyrosine kinases like Abl, Src and Lyn are expressed and purified from E. coli and purified as described in 3.4.8 to 3.4.11 or sold from commercial suppliers. p27 was phosphorylated in vitro by mixing recombinant p27 and the appropriate tyrosine kinase in a buffer containing 25 uM ATP, 200 mM Tris-HCl pH 7.2 and 75 mM MgCl2 as described in 3.4.13.

The isolated polypeptide or peptide fragment according to the invention can occur in natural allelic variations which differ from individual to individual. Such variations of the amino acids are usually amino acid substitutions. However, they may also be deletions, insertions or additions of amino acids to the total sequence. The polypeptide or peptide fragment according to the invention—depending, both in respect of the extent and type, on the cell and cell type in which it is expressed—can be in glycosylated or non-glycosylated form. This will be described in more detail below.

“Polypeptide with p27 activity” means also proteins with minor amino acid variations but with substantially the same activity. Substantially the same means that the activities are of the same biological properties and the polypeptides show (at least 80%, preferably more than 95%) homology or preferably identity in amino acid sequence. Homology can be examined by using the BLAST algorithm described by Altschul, S. F., et al., Nucleic Acids Res. 25 (1997) 3389-3402. This is also described in more detail below.

The polypeptide or peptide fragment according to the invention can be produced by recombinant means in host cells using an expression vector or can be produced synthetically. p27 polypeptide is obtained when it is produced recombinantly in prokaryotes. The p27 polypeptide or peptide fragment can be purified after recombinant production by affinity chromatography using known protein purification techniques including immunoprecipitation, gel filtration, ion exchange chromatography, chromato-focussing, isoelectric focussing, selective precipitation, electrophoresis or the like. This will be described in more detail below.

One aspect of the invention concerns isolated phosphorylated or phosphorylable proteins encoded by the nucleic acid sequence of the invention and biologically active portions thereof, as well as polypeptide fragments suitable for use as immunogens to raise antibodies directed against a polypeptide encoded by the nucleic acid sequence. In one embodiment the native polypeptide encoded by the nucleic acid sequence can be isolated from cells or tissue sources by an appropriate purification scheme using standard protein purification techniques. In another embodiment a polypeptide or peptide fragment encoded by the nucleic acid sequence of the invention is produced by recombinant DNA techniques. Alternative to recombinant expression, a polypeptide or peptide fragment encoded by the nucleic acid sequence according to the invention can be synthesized chemically using standard peptide synthesis techniques.

An “isolated” or “purified” polypeptide or peptide fragment or biologically active portion thereof is substantially free of cellular material or other contaminating proteins from the cell or tissue source from which the protein is derived or substantially free of chemical precursors or other chemicals when chemically synthesized. The term “substantially free of cellular material” includes preparations of polypeptide or peptide fragment in which the polypeptide or peptide fragment is separated from cellular components of the cells from which it is isolated or recombinantly produced. Thus a protein that is substantially free of cellular material includes preparations of protein having less than about 30%, 20%, 10%, or 5% (by dry weight) of heterologous protein (also referred to herein as a “contaminating protein”).

When the polypeptide or peptide fragment or biologically active portion thereof is recombinantly produced it is also preferably substantially free of culture medium i. e. culture medium represents less than about 20%, 10% or 5% of the volume of the protein preparation. When the protein is produced by chemical synthesis, it is preferably substantially free of chemical precursors or other chemicals i. e. it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide or peptide fragment encoded by a nucleic acid molecule according to the invention include polypeptide or peptide fragment comprising amino acid sequences sufficiently identical to or derived from the amino acid sequence of the protein according to the invention which include fewer amino acids than the fill length protein and exhibit at least one activity of the corresponding full-length protein. Typically biologically active portions comprise a domain or motif with at least one activity of the corresponding protein. A biologically active portion of the polypeptide or peptide fragment of the invention can be a polypeptide which is for example 10, 25, 50, 100 or more amino acids in length depending on the total length of the polypeptide or peptide fragment

Moreover, other biologically active portions in which other regions of the polypeptide or peptide fragment are deleted can be prepared by recombinant techniques and evaluated for one or more of the functional activities of the native form of a polypeptide of the invention. Other useful proteins are substantially identical (e.g. at least about 40%, preferably 50%, 60%, 70%, 80%, 90%, 95%, or 99%) to one of these sequences and retain the functional activity of the polypeptide or peptide fragment of the corresponding naturally-occurring polypeptide or peptide fragment yet differ in amino acid sequence due to natural allelic variation or mutagenesis.

To determine the percent identity of two amino acid sequences or of two nucleic acids the sequences are aligned for optimal comparison purposes (e.g. gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e. % identity=of identical positions/total of positions (e.g. overlapping positions)×100). In one embodiment the two sequences are the same length. The determination of percent identity between two sequences can be accomplished using a mathematical algorithm. A preferred, non-limiting example of a mathematical algorithm utilized for the comparison of two sequences is the algorithm of Karlin, S., and Altschul, S. F., Proc. Natl. Acad. Sci. USA 87 (1990) 2264-2268, modified as in Karlin, S., and Altschul, S. F., Proc. Natl. Acad. Sci. USA 90 (1993) 5873-5877. Such an algorithm is incorporated into the NBLAST and XBLAST programs of Altschul, S. F., et al., J. Mol. Biol. 215 (1990) 403-410. BLAST nucleotide searches can be performed with the NBLAST program, score=100, word length=12 to obtain nucleotide sequences homologous to a nucleic acid molecules of the invention. BLAST protein searches can be performed with the XBLAST program, score=50, word length=3 to obtain amino acid sequences homologous to a protein molecule of the invention. To obtain gapped alignments for comparison purposes, Gapped BLAST can be utilized as described in Altschul, S. F., et al., Nucleic Acids Res. 25 (1997) 3389-3402. Alternatively, PSI-Blast can be used to perform an iterated search which detects distant relationships between molecules. When utilizing BLAST, Gapped BLAST and PSI-Blast programs the default parameters of the respective programs (e.g., XBLAST and NBLAST) can be used. See http://www.ncbi.nlm.nih.gov. Another preferred non-limiting example of a mathematical algorithm utilized for the comparison of sequences is the algorithm of Myers and Miller, (1988) CABIOS or 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12 and a gap penalty of 4 can be used. Yet another useful algorithm for identifying regions of local sequence similarity and alignment is the FASTA algorithm as described in Pearson, W. R., and Lipman, D. J., Proc. Natl. Acad. Sci. USA 85 (1988) 2444-2448. When using the PASTA algorithm for comparing nucleotide or amino acid sequences, a PAM120 weight residue table can for example be used with a k-tuple value of 2. The percent identity between two sequences can be determined using techniques similar to those described above with or without allowing gaps. In calculating percent identity only exact matches are counted.

The invention also provides chimeric or fusion polypeptides or proteins corresponding to the polypeptide or peptide fragment according to the invention. As used herein, a “chimeric protein” or “fusion protein” comprises all or part (preferably a biologically active part) of the polypeptide or peptide fragment according to the invention operably linked to a heterologous polypeptide (i e. a polypeptide other than the polypeptide encoded by the nucleic acid molecule). Within the fusion protein the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention. One useful fusion protein is a GST fusion protein in which a polypeptide according to the invention is fused to the carboxyl terminus of GST sequences. Such fusion proteins can facilitate the purification of a recombinant polypeptide or peptide fragment of the invention. In another embodiment the fusion protein contains a heterologous signal sequence at its amino terminus. For example the native signal sequence of a polypeptide according to the invention can be removed and replaced with a signal sequence from another protein. For example the gp67 secretory sequence of the baculovirus envelope protein can be used as a heterologous signal sequence (Ausubel et al., ed., Current Protocols in Molecular Biology, John Wiley & Sons, N.Y., 1992). Other examples of eukaryotic heterologous signal sequences include the secretory sequences of melittin and human placental alkaline phosphatase (Stratagene; La Jolla, Calif.). In yet another example useful prokaryotic heterologous signal sequences include the phoA secretory signal (Sambrook et al., ed., Molecular Cloning: A Laboratory Manual, 2^{nd} ed., Cold Spring Harbor Laboratory Press, Cold Spring Harbor, N.Y., 1989) and the protein A secretory signal (Pharmacia Biotech; Piscataway, N.J.). Cell permeable fusion proteins to deliver mutant p27 or CKI domains into cells may also be generated. These fusions may include a peptide from the TAT protein of HIV, a domain of the antennapedia protein (Antp) from drosophila or HSV VP22 protein from HSV. An example of cell permeable p27fusion proteins is described in Nagahara, H., et al., Nat. Med. 4 (1998) 1449-1452. The TAT peptide used here is the N-terminal tag YGRKKRRQRRR.

In yet another embodiment the fusion protein is an immunoglobulin fusion protein in which all or part of a polypeptide according to the invention is fused to sequences derived from a member of the immunoglobulin protein family. The immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment the fusion gene can be synthesized by conventional techniques including automated DNA synthesizers.

Alternatively the PCR amplification of gene fragments can be carried out using anchor primers which give rise to complementary overhangs between two consecutive gene fragments which can subsequently be annealed and re-amplified to generate a chimeric gene sequence (see e.g. Ausubel et al., ed., Current Protocols in Molecular Biology, John Wiley & Sons, N.Y., 1992). Moreover, many expression vectors are commercially available that already encode a fusion moiety (e.g. a GST polypeptide).

A nucleic acid encoding a polypeptide of the invention can be cloned into such an expression vector such that the fusion moiety is linked in-frame to the polypeptide or peptide fragment of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus the invention pertains to the described polypeptides having a signal sequence, as well as to polypeptides from which the signal sequence has been proteolytically cleaved (i. e. the cleavage products). In one embodiment a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed and the signal sequence is subsequently or concurrently cleaved. The protein can then be readily purified from the extracellular medium by art recognized methods.

Alternatively the signal sequence can be linked to the protein of interest using a sequence which facilitates purification such as with a GST domain.

The present invention also pertains to variants of the polypeptide or peptide fragment encoded by nucleic acid molecule of the invention. Such variants have an altered amino acid sequence which can function as either agonists (mimetics) or as antagonists. Variants can be generated by mutagenesis e.g. discrete point mutation or truncation. Preferred variants are those which differ by 2, preferably one amino acid from the polypeptide or peptide fragment according to the invention. To differ means that two, preferably one amino acid is different that in the nucleic acid sequence referred to. An agonist can retain substantially the same, or a subset of the biological activities of the naturally occurring protein. An antagonist of a protein can inhibit one or more of the activities of the naturally occurring form of the protein by for example competitively binding to a downstream or upstream member of a cellular signalling cascade which includes the protein of interest. Thus specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Further variants of a polypeptide or peptide fragment of the invention which function as either agonists (mimetics) or as antagonists can be identified by screening combinatorial libraries of mutants e.g. truncation mutants of the protein of the invention for agonist or antagonist activity. In one embodiment a variegated library of variants is generated by combinatorial mutagenesis at the nucleic acid level and is encoded by a variegated gene library. A variegated library of variants can be produced by for example enzymatically ligating a mixture of synthetic oligonucleotides into gene sequences such that a degenerate set of potential protein sequences is expressible as individual polypeptides or alternatively as a set of larger fusion proteins (e.g. for phage display). There are a variety of methods which can be used to produce libraries of potential variants of the polypeptides of the invention from a degenerate oligonucleotide sequence. Methods for synthesizing degenerate oligonucleotides are known in the art (see e.g. Narang, Tetrahedron 39 (1983) 3; Itakura, K., et al., Annu. Rev. Biochem. 53 (1984) 323-356; Itakura, K., et al., Science 198 (1977) 1056-1063; Ike, Y., et al., Nucleic Acid Res. 11 (1983) 477-488).

In addition libraries of fragments of the coding sequence of a polypeptide encoded by the nucleic acid molecule according to the invention can be used to generate a variegated population of polypeptides for screening and subsequent selection of variants. A library of coding sequence fragments can for example be generated by treating a double-stranded PCR fragment of the coding sequence of interest with a nuclease under conditions wherein nicking occurs only about once per molecule, denaturing the double stranded DNA, renaturing the DNA to form double-stranded DNA which can include sense/antisense pairs from different nicked products, removing single stranded portions from reformed duplexes by treatment with S1 nuclease and ligating the resulting fragment library into an expression vector. By this method an expression library can be derived which encodes amino terminal and internal fragments of various sizes of the protein of interest.

Several techniques are known in the prior art for screening gene products of combinatorial libraries made by point mutations or truncation and for screening cDNA libraries for gene products having a selected property. The most widely used techniques which are amenable to high through-put analysis for screening large gene libraries typically include cloning the gene library into replicable expression vectors, transforming appropriate cells with the resulting library of vectors and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin, A. P., and Youvan, D. C., Proc. Natl. Acad. Sci. USA 89 (1992) 7811-7815; Delagrave, S., et al., Prot. Eng. 6 (1993) 327-331).

An isolated polypeptide according to the invention or a fragment thereof can be used as an immunogen to generate antibodies using standard techniques for polyclonal and monoclonal antibody preparation. The full-length polypeptide or protein can be used or alternatively the invention provides antigenic peptide fragments for use as immunogens. The antigenic peptide fragment of a polypeptide of the invention comprises at least 6, 8 (preferably 10, 15, 20, or 30 or more) amino acid residues of the amino acid sequence of the polypeptide of the invention and encompasses an epitope of the protein comprising the phosphorylated amino acid residues Y 88 and/or Y 89 such that an antibody raised against the peptide forms a specific immune complex with a protein according to the invention. Preferred epitopes encompassed by the antigenic peptide are regions that are located on the surface of the protein e.g. hydrophilic regions. Hydrophobicity sequence analysis, hydrophilicity sequence analysis or similar analyses can be used to identify hydrophilic regions. An immunogen typically is used to prepare antibodies by immunizing a suitable (i.e. immunocompetent) subject such as a rabbit, goat, mouse or other mammal or vertebrate. An appropriate immunogenic preparation can for example contain recombinantly-expressed or chemically-synthesized polypeptide. The preparation can further include an adjuvant, such as Freund's complete or incomplete adjuvant or a similar immunostimulatory agent.

Therefore, in an embodiment of the invention, a polypeptide according to the invention or a peptide fragment, in particular the phosphorylated proteins or peptide fragments, can be used as an immunogen to generate or produce antibodies using standard techniques for polyclonal and monoclonal antibody preparation. The standard techniques are described herein and are known to an expert skilled in the art.

Accordingly another aspect of the invention pertains to antibodies directed against a polypeptide of the invention. The terms “antibody” and “antibody substance” as used interchangeably herein refer to immunoglobulin molecules and immunologically active portions of immunoglobulin molecules i. e. molecules that contain an antigen binding site which specifically binds an antigen, such as a polypeptide of the invention e.g. an epitope of a polypeptide of the invention. A molecule which specifically binds to a given polypeptide of the invention is a molecule which binds the polypeptide, but does not substantially bind other molecules in a sample e.g. a biological sample which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition” as used herein refers to a population of antibody molecules that contain only one species of an antigen binding site capable of immunoreacting with a particular epitope.

Polyclonal antibodies can be prepared as described above by immunizing a suitable subject with a polypeptide or peptide fragment of the invention as an immunogen as also shown in the example part of this application. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as for example immunogen compositions made using a non-human host cell for recombinant expression of a polypeptide of the invention. In such a manner the only human epitope or epitopes recognized by the resulting antibody compositions raised against this immunogen will be present as part of a polypeptide or polypeptides of the invention.

The antibody titre in the immunized subject can be monitored over time by standard techniques such as with an enzyme linked immunosorbent assay (ELISA) using immobilized polypeptide. If desired the antibody molecules can be harvested or isolated from the subject (e.g. from the blood or serum of the subject) and further purified by well-known techniques such as protein A chromatography to obtain the IgG fraction. Alternatively antibodies specific for a protein or polypeptide of the invention can be selected or (e.g. partially purified) or purified by e.g. affinity chromatography.

For example a recombinantly expressed and purified (or partially purified) polypeptide or peptide fragment of the invention is produced as described herein and covalently or non-covalently coupled to a solid support such as for example a chromatography column. The column can then be used to affinity purify antibodies specific for the proteins of the invention from a sample containing antibodies directed against a large number of different epitopes, thereby generating a substantially purified antibody composition i.e. one that is substantially free of contaminating antibodies. By a substantially purified antibody composition is meant in this context that the antibody sample contains at most only 30% (by dry weight) of contaminating antibodies directed against epitopes other than those of the desired protein or polypeptide of the invention and preferably at most 20% yet more preferably at most 10% and most preferably at most 5% (by dry weight) of the sample is contaminating antibodies. A purified antibody composition means that at least 99% of the antibodies in the composition are directed against the desired protein or polypeptide of the invention.

At an appropriate time after immunization e.g. when the specific antibody titres are highest antibody-producing cells can be obtained from the subject and used to prepare monoclonal antibodies by standard techniques, such as the hybridoma technique originally described by Kohler, G., and Milstein, C., Nature 256 (1975) 495-497 the human B cell hybridoma technique (see Kozbor et al., Immunol. Today 4 (1983) 72), the EBV hybridoma technique (see Cole et al., In: Monoclonal Antibodies and Cancer Therapy, Alan R Liss, Inc., 1985, pp. 77-96) or trioma techniques. The technology for producing hybridomas is well known (see generally Coligan et al., ed., Current Protocols in Immunology, John Wiley & Sons, N.Y., 1994). Hybridoma cells producing a monoclonal antibody of the invention are detected by screening the hybridoma culture supernatants for antibodies that bind the polypeptide of interest e.g. using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g. an antibody phage display library) with the polypeptide of interest. Kits for generating and screening phage display libraries are commercially available (e.g. the Pharmacia Recombinant Phage Antibody System, Catalog No. 27-9400-01; end the Stratagene Su′ 4P Phage Display Kit, Catalog No. 240612). Additionally examples of methods and reagents particularly amenable for use in generating and screening antibody display library can for example be found in U.S. Pat. No. 5,223,409; WO 92/18619; WO 91/17271; WO 92/20791; WO 92/15679; WO 93/01288; WO 92/01047; WO 92/09690; WO 90/02809; Fuchs, P., et al., Bio/Technology 9 (1991)1370-1372; Hay, B. N., et al., Hum. Antibod. Hybridomas 3 (1992) 81-85; Huse, W. D., et al., Science 246 (1989) 1275-1281; Griffiths, A. D., et al., EMBO J. 12 (1993) 725-734.

An antibody directed against a polypeptide according to the invention (e.g. a monoclonal antibody) can be used to isolate the polypeptide by standard techniques, such as affinity chromatography or immunoprecipitation.

Moreover, such an antibody can be used to detect the polypeptide or peptide fragment (e.g. in a cellular lysate or cell supernatant) in order to evaluate the level and pattern of expression thereof. The antibodies can also be used diagnostically to monitor protein levels in tissues or body fluids (e.g. in a tissue-associated body fluid) as part of a clinical testing procedure e.g. to for example determine the efficacy of a given treatment regimen.

Detection can be facilitated by coupling the antibody to a detectable substance. Examples of detectable substances include various enzymes, prosthetic groups, fluorescent materials, luminescent materials, bioluminescent materials and radioactive materials. Examples of suitable enzymes include horseradish peroxidase, alkaline phosphatase, 6-galactosidase or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin and aequorin and examples of suitable radioactive material include I, P, S, C or H.

Accordingly in one aspect the invention provides substantially purified antibodies or fragments thereof and non-human antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 6, preferably 10 amino acid residues of an amino acid sequence of the present invention being preferably phosphorylated, an amino acid sequence which is at least 95% identical to the amino acid sequence of the present invention (wherein the percent identity is determined using the ALIGN program of the GCG software package with a PAM120 weight residue table, a gap length penalty of 12 and a gap penalty of 4 and an amino acid sequence which is encoded by a nucleic acid molecule which hybridizes to a nucleic acid molecule consisting of the nucleic acid molecules of the present invention or a complement thereof under conditions of hybridization of 6×SSC at 45° C. and washing in 0.2×SSC, 0.1% SDS at 65° C. In various embodiments the substantially purified antibodies of the invention or fragments thereof can be human, non-human, chimeric and/or humanized antibodies.

In another aspect the invention provides non-human antibodies or fragments thereof which antibodies or fragments specifically bind to a polypeptide comprising an amino acid sequence selected from the group consisting of the amino acid sequence of the present invention, an amino acid sequence encoded by the cDNA of the present invention, a fragment of at least 6, preferably 10 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12 and a gap penalty of 4 and an amino acid sequence which is encoded by a nucleic acid molecule which hybridizes to a nucleic acid molecule according to the invention or a complement thereof under conditions of hybridization of 6×SSC at 45° C. and washing in 0.2×SSC, 0.1% SDS at 65° C. Such non-human antibodies can be goat, mouse, sheep, horse, donkey, chicken, rabbit or rat antibodies. Alternatively the non-human antibodies of the invention can be chimeric and/or humanized antibodies. In addition the non human antibodies of the invention can be polyclonal antibodies or monoclonal antibodies.

In still a further aspect the invention provides monoclonal antibodies or fragments thereof which antibodies or fragments specifically bind to a peptide/polypeptide comprising an amino acid sequence selected from the group consisting of the amino acid sequences of the present invention, an amino acid sequence encoded by the cDNA of the present invention, a fragment of at least 6, preferably 10 amino acid residues of an amino acid sequence of the present invention, an amino acid sequence which is at least 95% identical to an amino acid sequence of the present invention (wherein the percent identity is determined using the ALIGN program of the GCG software package with a PAM120 weight residue table), a gap length penalty of 12 and a gap penalty of 4 and an amino acid sequence which is encoded by a nucleic acid molecule which hybridizes to a nucleic acid molecule consisting of the nucleic acid molecules of the present invention or a complement thereof under conditions of hybridization of 6×SSC at 45° C. and washing in 0.2×SSC, 0.1% SDS at 65° C. The monoclonal antibodies can be human, humanized, chimeric and/or non-human antibodies.

Any of the antibodies of the invention can be conjugated to a detectable substance. Non-limiting examples of detectable substances that can be conjugated to the antibodies of the invention are an enzyme, a prosthetic group, a fluorescent material, a luminescent material, a bioluminescent material and a radioactive material.

The invention also provides a kit containing an antibody of the invention conjugated to a detectable substance and instructions for use.

Still another aspect of the invention is a method of making an antibody that specifically recognizes a polypeptide or peptide fragment of the present invention, the method comprising immunizing a mammal or a vertebrate with a polypeptide. The polypeptide used as an immunogen comprises an amino acid sequence selected from the group consisting of the amino acid sequence of the present invention, an amino acid sequence encoded by the cDNA of the nucleic acid molecules of the present invention, a fragment of at least 6, preferably 10 amino acid residues of the amino acid sequence of the present invention, an amino acid sequence which is at least 95% identical to the amino acid sequence of the present invention (wherein the percent identity is determined using the ALIGN program of the GCG 3s software package with a PAM120 weight residue table, a gap length penalty of 12 and a gap penalty of 4 and an amino acid sequence which is encoded by a nucleic acid molecule which hybridizes to a nucleic acid molecule consisting of the nucleic acid molecules of the present invention or a complement thereof under conditions of hybridization of 6×SSC at 45° C. and washing in 0.2×SSC, 0.1% SDS at 65° C.

After immunization a sample is collected from the animal that contains an antibody that specifically recognizes the polypeptide. The polypeptide is preferably produced recombinantly using a non-human host cell. Optionally the antibodies can be further purified from the sample using techniques well known to a person skilled in the art.

The method can further comprise producing a monoclonal antibody-producing cell from the cells of the animal. Optionally antibodies are collected from the antibody-producing cell.

In summary, the invention provides an isolated antibody which specifically binds to a polypeptide according to the invention or to a peptide fragment according to the the invention and which has less than 10%, preferably 5%, more preferably 2%, 1%, 0.5%, 0.1% or 0.01% or even no cross reactivity with the corresponding non-phosphorylated polypeptide or corresponding non-phosphorylated peptide fragment. The cross reactivity can be determined by enzyme immuno assay or radio immuno assay as known to the expert skilled in the art and described in the application. The antibody may be a polyclonal antibody but is preferably a monoclonal antibody. The polyclonal antibody serum may be purified from cross-reacting antibodies by binding the non-phosphorylated antigen to which some antibodies might cross-react to a column and isolating the non-binding (more specific) polyclonal antibodies.

The preferred hybridoma cell lines according to the invention Mab<p27kip1>15 and Mab<p27kip1>388 were deposited on Oct. 6, 2004 under the Budapest Treaty on the international recognition of the deposit of microorganisms for the purposes of patent procedure, with Deutsche Sammlung von Mikroorganismen und Zelliculturen GmbH (DSMZ), Germany:

| Cell line | Deposition No. | Date of Deposit |
|---|---|---|
| Mab<p27kip1>15 | DSM ACC | Oct. 6, 2004 |
| Mab<p27kip1>388 | DSM ACC | Oct. 6, 2004 |

The antibodies obtainable or produced from said cell lines as well as the hybridoma cell lines themselves are preferred embodiments of the invention.

II. Nucleic acids

One aspect of the invention pertains to isolated nucleic acid molecules that encode the polypeptide or peptide fragment according to the invention, in particular the non-phosphorylable analogues thereof, or the antibody according to the invention and can therefore be used for the production of the polypeptide or antibody according to the invention or precursors thereof. In another embodiment of the invention a DNA vector is provided comprising a nucleic acid molecule encoding a polypeptide according to the invention or a peptide fragment according to the invention.

Such nucleic acid molecules comprise sequences of RNA transcripts or portions of such transcripts. As used herein, the term “nucleic acid molecule” is intended to include DNA molecules (e.g. cDNA or genomic DNA) and RNA molecules (e.g. mRNA) and analogues of the DNA or RNA generated using nucleotide analogues. The nucleic acid molecule can be single-stranded or double-stranded, but preferably is double-stranded DNA.

The invention also encompasses nucleic acids which differ from that of the nucleic acids described herein, but which produce the same phenotypic effect such as an allelic variant. These altered but phenotypically equivalent nucleic acids are referred to as “equivalent nucleic acids.” This invention also encompasses nucleic acids characterized by changes in non-coding regions that do not alter the polypeptide produced therefrom when compared to the polynucleotide herein. This invention further encompasses nucleic acids which hybridize to the polynucleotides of the subject invention under conditions of moderate or high stringency. Alternatively the polynucleotides are at least 85% or at least 90% or more preferably greater or equal to 95% identical as determined by a sequence alignment program when run under default parameters.

An “isolated” nucleic acid molecule is one which is separated from other nucleic acid molecules which are present in the natural source of the nucleic acid molecule. Preferably an “isolated” nucleic acid molecule comprises a protein-coding sequence and is free of sequences which naturally flank the coding sequence in the genomic DNA of the organism from which the nucleic acid is derived. For example in various embodiments the isolated nucleic acid molecule can contain less than about 5 kB 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover an “isolated” nucleic acid molecule such as a cDNA molecule can be substantially free of other cellular material or culture medium when produced by recombinant techniques or substantially free of chemical precursors or other chemicals when chemically synthesized.

A nucleic acid molecule of the present invention can be isolated using standard molecular biology techniques. A nucleic acid molecule of the present invention also encompasses the nucleic acid molecules which can be isolated using standard hybridization and cloning techniques (e.g. as described in Sambrook et al., Molecular Cloning: A Laboratory Manual, 2nd ed., Cold Spring Harbor Laboratory Press, Cold Spring Harbor, N.Y., 1989).

The nucleic acid molecule according to the invention can be amplified using cDNA, mRNA or genomic DNA as a template and appropriate oligonucleotide primers according to standard PCR amplification techniques. The nucleic acid amplified in this manner can be cloned into an appropriate vector and characterized by DNA sequence analysis. Furthermore oligonucleotides corresponding to all or a portion of the nucleic acid molecule according to the invention can be prepared by standard synthetic techniques e.g. using an automated DNA synthesizer. In another preferred embodiment an isolated nucleic acid molecule of the invention comprises a nucleotide sequence of a RNA transcript according to the invention or a complement of said sequence. A nucleic acid molecule which is complementary to a given nucleotide sequence is one which is sufficiently complementary to the given nucleotide sequence that it can hybridize to the given nucleotide sequence thereby forming a stable duplex.

Moreover a nucleic acid molecule of the invention can comprise only a portion of the nucleotide sequence (RNA or cDNA) of a RNA transcript or a complement of said sequence. Such nucleic acids can for example be used as a probe or primer. The probe/primer is typically used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of the nucleic acid molecule according to the invention.

Probes based on the sequence of the nucleic acid sequence according to the invention can be used to detect transcripts or genomic sequences of the nucleic acid molecule according to the invention. The probe comprises a label group attached thereto e.g. a radioisotope, a fluorescent compound, an enzyme, or an enzyme co-factor. Such probes can generally be used as part of a diagnostic test kit for identifying cells or tissues which mix-express the protein, such as by measuring levels of a nucleic acid molecule encoding the protein in a sample of cells from a subject e.g. detecting mRNA levels or determining whether a gene encoding the protein has been mutated or deleted.

The invention further encompasses nucleic acid molecules that differ due to degeneracy of the genetic code from the nucleotide sequence of nucleic acids encoding a protein which corresponds to the nucleic acid molecule according to the invention and thus encode the same protein.

It will be appreciated by a person skilled in the art that DNA sequence polymorphisms that lead to changes in the amino acid sequence can exist within a population (e.g. the human population). Such genetic polymorphisms can exist among individuals within a population due to natural allelic variation. An allele is one of a group of genes which occur alternatively at a given genetic locus. In addition it will be appreciated that DNA polymorphisms that affect RNA expression levels can also exist that may affect the overall expression level of that gene (e.g. by affecting regulation or degradation). As used herein the phrase “allelic variant” refers to a nucleotide sequence which occurs at a given locus or to a polypeptide encoded by the nucleotide sequence.

As used herein the terms “gene” and “recombinant gene” refer to nucleic acid molecules comprising an open reading frame encoding a polypeptide according to the invention. Such natural allelic variations can typically result in 0.1-0.5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

As used herein the term “hybridizes under stringent conditions” is intended to describe conditions for hybridization and washing under which nucleotide sequences at least 75% (80%, 85% preferably 90%) identical to each other typically remain hybridized to each other. Such stringent conditions are known to persons skilled in the art and can be found in sections 6.3.1-6.3.6 of Current Protocols in Molecular Biology, John Wiley & Sons, N.Y. (1989). A preferred non-limiting example of stringent hybridization conditions for annealing two single-stranded DNA each of which is at least about 100 bases in length and/or for annealing a single-stranded DNA and a single-stranded RNA each of which is at least about 100 bases in length, means hybridization at 65° C. in a hybridization buffer consisting of 250 mmol/l sodium phosphate buffer pH 7.2, 7% (w/v) SDS, 1% (w/v) BSA, 1 mmol/l EDTA and 0.1 mg/ml single-stranded salmon sperm DNA. A final wash is performed at 65° C. in 125 mmol/l sodium phosphate buffer pH 7.2, 1 mmol/l EDTA and 1% (w/v) SDS. Further preferred hybridization conditions are taught in Lockhart, D. J., et al., Nat Biotechnol. 14 (1996) 1675-1680; Breslauer, K. J., et al., Proc. Natl. Acad. Sci. USA 83 (1986) 3746-3750; van Ness, J., and Chen, L., Nucleic Acids Res. 19 (1991) 5143-5151; McGraw, R. A., et al., BioTechniques 8 (1990) 674-678; and Milner, N., et al., Nat. Biotechnol. 15 (1997) 537-541, all expressly incorporated by reference. In addition to naturally-occurring allelic variants of the nucleic acid sequence according to the invention that can exist in the population, the skilled artisan will further appreciate that sequence changes can be introduced by mutation thereby leading to changes in the amino acid sequence of the encoded protein, without altering the biological activity of the protein encoded thereby. For example one can make nucleotide substitutions leading to amino acid substitutions at “non-essential” amino acid residues. A “non essential” amino acid residue is a residue that can be altered from the wild-type sequence without altering the biological activity, whereas an “essential” amino acid residue is required for biological activity. For example amino acid residues that are not conserved or only semi-conserved among homologues of various species may be non essential for activity and thus would be likely targets for alteration. Alternatively amino acid residues that are conserved among the homologues of various species (e.g. murine and human) may be essential for activity and thus would not be likely targets for alteration. Accordingly another aspect of the invention pertains to nucleic acid molecules encoding a polypeptide of the invention that contain changes in amino acid residues that are not essential for activity. Such polypeptides differ in amino acid sequence from the naturally-occurring proteins encoded by the nucleic acid sequence according to the invention, yet retain biological activity. In one embodiment such a protein has an amino acid sequence that is at least about 40% identical, 50%, 60%, 70%, 80%, 90%, 95%, or 98% identical to the amino acid sequence of one of the proteins according to the invention. An isolated nucleic acid molecule encoding a variant protein can be created by introducing one or more nucleotide substitutions, additions or deletions into the nucleotide sequence of nucleic acids of the invention such that one or more amino acid residue substitutions, additions or deletions are introduced into the encoded protein.

Mutations can be introduced by standard techniques such as site-directed mutagenesis and PCR-mediated mutagenesis. Conservative amino acid substitutions are preferably made at one or more predicted non-essential amino acid residues. A “conservative amino acid substitution” is one in which the amino acid residue is replaced with an amino acid residue having a similar side chain. Families of amino acid residues having similar side chains have been deemed in the art. These families include amino acids with basic side chains (e.g. lysine, arginine, histidine) acidic side chains (e.g. aspartic acid, glutamic acid) uncharged polar side chains (e.g. glycine, asparagine, glutamine, serine, threonine, tyrosine, cysteine) non-polar side chains (e.g. alanine, valine, leucine, isoleucine, proline, phenylalanine, methionine, tryptophan) beta-branched side chains (e.g. threonine, valine, isoleucine) and aromatic side chains (e.g. tyrosine, phenylalanine, tryptophan, histidine). Alternatively mutations can be introduced randomly along all or part of the coding sequence such as by saturation mutagenesis and the resultant mutants can be screened for biological activity to identify mutants that retain activity. Following mutagenesis the encoded protein can be expressed recombinantly and the activity of the protein can be determined.

III. Recombinant Expression Vectors and Host Cells

Another aspect of the invention pertains to vectors preferably expression vectors, containing a nucleic acid sequence encoding a polypeptide according to the invention (or a portion of such a polypeptide as the peptide fragment).

As used herein the term “vector” refers to a nucleic acid molecule capable of transporting another nucleic acid to which it has been linked. One type of vector is a “plasmid” which refers to a circular double stranded DNA loop into which additional DNA segments can be ligated. Another type of vector is a viral vector wherein additional DNA segments can be ligated into the viral genome. Certain vectors are capable of autonomous replication in a host cell into which they are introduced (e.g. bacterial vectors having a bacterial origin of replication and episomal mammalian vectors). Other vectors (e.g. non-episomal mammalian vectors) are integrated into the genome of a host cell upon introduction into the host cell and thereby are replicated along with the host genome. Moreover, certain vectors namely expression vectors are capable of directing the expression of genes to which they are operably linked. In general expression vectors of utility in recombinant DNA techniques are often in the form of plasmids (vectors). However, the invention is intended to include such other forms of expression vectors such as viral vectors (e. g. replication defective retroviruses, adenoviruses and adeno-associated viruses) which serve equivalent functions. The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g. in an in vitro transcription/translation system or in a host cell when the vector is introduced into the host cell). The term “regulatory sequence” is intended to include promoters, enhancers and other expression control elements (e.g. polyadenylation signals). Such regulatory sequences are described for example in Goeddel, Methods in Enzymology. Gene Expression Technology, Vol. 185, Academic Press, San Diego, Calif., 1991. Regulatory sequences include those which direct constitutive expression of a nucleotide sequence in many types of host cell and those which direct expression of the nucleotide sequence only in certain host cells (e.g. tissue-specific regulatory sequences). It will be appreciated by persons skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide according to the invention in prokaryotic (e.g. E. coli) or eukaryotic cells (end insect cells using baculovirus expression vectors, yeast cells or mammalian cells). Suitable host cells are discussed further in Goeddel, supra Alternatively the recombinant expression vector can be transcribed and translated in vitro for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein usually to the amino terminus of the recombinant protein. Such fusion vectors typically serve three purposes: 1) to increase expression of recombinant protein; 2) to increase the solubility of the recombinant protein and 3) to aid in the purification of the recombinant protein by acting as a ligand in affinity purification. Often in fusion expression vectors a proteolytic cleavage site is introduced at the junction of the fusion moiety and the recombinant protein to enable separation of the recombinant protein from the fusion moiety subsequent to purification of the fusion protein. Such enzymes and their cognate recognition sequences include Factor Xa, thrombin and enterokinase.

Typical fusion expression vectors include pGEX (Pharmacia Biotech Inc; Smith, D. B., and Johnson, K S., Gene 67 (1988) 31-40), pMAL (New England Biolabs, Beverly, Mass.) and pRIT5 (Phannacia, Piscataway, N.J.) which fuse glutathione S-transferase (GST), maltose E binding protein or protein A respectively to the target recombinant protein. Examples of suitable inducible non-fusion E. coli expression vectors include pTrc (Amann, E., et al., Gene 69 (1988) 301-315) and pET 1 Id (Studier et al., In: Gene Expression Technology: Methods in Enzymology, Vol. 85, Academic Press, San Diego, Calif., 1991, pp. 60-89). Target gene expression from the pTrc vector relies on host RNA polymerase transcription from a hybrid trp-lac fusion promoter. Target gene expression from the pET 1 Id vector relies on transcription from a T7 gulO-lac fusion promoter mediated by a co-expressed viral RNA polymerase (T7 gal). This viral polymerase is supplied by host strains BL21(DE3) or HMS 174(DE3) from a resident prophage harboring a T7 gal gene under the transcriptional control of the lacUV promoter.

One strategy to maximize recombinant protein expression in E. coli is to express the protein in a host bacterium with an impaired capacity to proteolytically cleave the recombinant protein (Gottesman, In: Gene Expression Technology: Methods in Enzymology, Vol. 185, Academic Pres, San Diego, Calif., 1990, pp. 119-128). Another strategy is to alter the nucleic acid sequence of the nucleic acid to be inserted into an expression vector so that the individual codons for each amino acid are those preferentially utilized in E. coli (Wada, K., et al., Nucleic Acids Res. 20 (1992) 2111-2118). Such alteration of nucleic acid sequences of the invention can be carried out by standard DNA synthesis techniques. In another embodiment the expression vector is a yeast expression vector.

Examples of vectors for expression in yeast S. cerevisiae include pYepSecl (Baldari, C., et al., EMBO J. 6 (1987) 229-234), pMFa (Kuzjan, J., and Herskowitz, I., Cell 30 (1982) 933-943), pJRY88 (Schultz, L. D., et al., Gene 54 (1987) 113-123), pYES2 (Invitrogen Corporation, San Diego, Calif.) and pPicZ (Invitrogen Corp, San Diego, Calif.).

Alternatively the expression vector is a baculovirus expression vector. Baculovirus vectors available for expression of proteins in cultured insect cells (e.g. Sf9 cells) include the pAc series (Smith, G. E., et al., Mol. Cell Biol. 3 (1983) 2156-2165) and the pVL series (Luckow, V. A., and Summers, M. D., Virology 170 (1989) 31-39).

In yet another embodiment the protein according to the invention is expressed in mammalian cells using a mammalian expression vector. Examples of mammalian expression vectors include pCDM8 (Seed, B., Nature 329 (1987) 840-842) and pMT2NOPC (Kaufman, R. J., et al., EMBO J. 6 (1987) 187-193). When used in mammalian cells the expression vector's control functions are often provided by viral regulatory elements.

For example commonly used promoters are derived from polyoma, adenovirus 2, cytomegalovirus and Simian Virus 40. For other suitable expression systems for both prokaryotic and eukaryotic cells see chapters 16 and 17 of Sambrook et al., supra.

In another embodiment the recombinant mammalian expression vector is capable of directing expression of the nucleic acid preferentially in a particular cell type (e.g. tissue-specific regulatory elements are used to express the nucleic acid). Tissue specific regulatory elements are known in the art. Non-limiting examples of suitable tissue-specific promoters include the albumin promoter (liver-specific; Pinkert, C. A. et al., Genes Dev. 1 (1987) 268-277), lymphoid-specific promoters (Calame, K and Eaton, S., Adv. Immunol. 43 (1988) 235-275) in particular promoters of T cell receptors (Winoto, A., and Baltimore, D., EMBO J. 8 (1989) 729-733) and immunoglobulins (Banerji, J., et al., Cell 33 (1983) 729-740; Queen, C. and Baltimore, D., Cell 33 (1983) 741-748), neuron-specific promoters (e.g. the neurofilament promoter; Byrne, G. W., and Ruddle, F. H., Proc. Natl. Acad. Sci. USA 86 (1989) 5473-5477), pancreas-specific promoters (Edlund, T., et al., Science 230 (1985) 912-916) and mammary gland-specific promoters (e.g. milk whey promoter, U.S. Pat. No. 4,873,316 and EP-A 0 264 166). Developmentally regulated promoters are also encompassed for example the murine box promoters (Kessel, M., and Gruss, P., Science 249 (1990) 374-379) and the alpha-fetoprotein promoter (Camper, S. A., and Tilghman, S. M., Genes Dev. 3 (1989) 537-546).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences such progeny may not in fact be identical to the parent cell but are still included within the scope of the term as used herein. A host cell can be any prokaryotic (e.g. E. coli) or eukaryotic cell (e.g. insect cells, yeast or mammalian cells).

Vector DNA can be introduced into prokaryotic or eukaryotic cells via conventional transformation or transfection techniques. As used herein the terms “transformation” and “transfection” are intended to refer to a variety of art-recognized; techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra) and other laboratory manuals.

For stable transfection of mammalian cells it is known that depending upon the expression vector and transfection technique used only a small fraction of cells may integrate the foreign DNA into their genome. In order to identify and select these integrants a gene that encodes a “selectable marker” (SM) gene (e.g. for resistance to antibiotics) is generally introduced into the host cells along with the gene of interest.

Preferred SM genes include those which confer resistance to drugs such as G418, hygromycin and methotrexate. Cells stably transfected with the introduced nucleic acid can be identified by drug selection (e.g. cells that have incorporated the SM gene will survive while the other cells die).

A host cell of the invention such as a prokaryotic or eukaryotic host cell in culture can be used to produce a polypeptide according to the invention. Accordingly the invention further provides methods for producing a polypeptide according to the invention using the host cells of the invention. In one embodiment the method comprises culturing the host cell of invention (into which a recombinant expression vector encoding a polypeptide of the invention has been introduced) in a suitable medium such that the polypeptide according to the invention is produced. In another embodiment the method further comprises isolating the polypeptide from the medium or the host cell. The host cells of the invention can also be used to produce nonhuman transgenic animals. For example in one embodiment a host cell of the invention is a fertilized oocyte or a(n embryonic) stem cell into which a sequences encoding a polypeptide according to the invention have been introduced. Such host cells can then be used to create non-human transgenic animals in which exogenous sequences encoding the polypeptide according to the invention have been introduced into their genome or homologous recombinant animals in which endogenous gene(s) encoding a polypeptide according to the invention have been altered.

IV. Predictive Medicine

The present invention pertains to the field of predictive medicine in which diagnostic assays, prognostic assays, pharmacogenomics and monitoring clinical trials are used for prognostic (predictive) purposes to thereby treat an individual prophylactically. Accordingly one aspect of the present invention relates to diagnostic assays for determining the level of the phosphorylated protein according to the invention. Such assays can be used for prognostic or predictive purposes to thereby prophylactically treat an individual prior to the metastasis of the cancer.

A particularly preferred embodiment of the invention in this context, is a method for the determination of the amount or presence of a polypeptide according to the invention or a peptide fragment according to the invention in a sample comprising the steps of
a) providing a sample suspected to contain the polypeptide or the peptide fragment,
b) incubating the sample in the presence of an antibody according to the invention, and
c) determining the binding product between the polypeptide and the antibody thereby concluding that the polypeptide or peptide fragment is present or thereby deriving the amount of the polypeptide or the peptide fragment.

A particularly other preferred embodiment of the invention in this context, is a method for the determination of the amount or presence of a polypeptide according to the invention or a peptide fragment according to the invention in a sample comprising the steps of
a) incubating a sample suspected to contain the polypeptide or the peptide fragment in the presence of an antibody according to the invention, and
b) determining the binding product between the polypeptide and the antibody thereby concluding that the polypeptide or peptide fragment is present or thereby deriving the amount of the polypeptide or the peptide fragment.

Another preferred embodiment is a method of determining whether or not a human cancer cell containing patient sample has potential for tumor progression, the method comprising comparing:
a) the level of expression a polypeptide according to the invention or a peptide fragment according to the invention in the patient sample, and
b) the normal level of expression the polypeptide or the peptide fragment in a sample from a control subject not afflicted with cancer,
   and a significant difference between the level of expression of the polypeptide or the peptide fragment in the patient sample and the normal level of the polypeptide or the peptide fragment in the sample from a control subject not afflicted with cancer is an indication that the patient sample has potential for tumor progression. The significant difference is an at least 1.5 fold difference or a less than 0.75 fold difference.

The normal level of expression the polypeptide or the peptide fragment can also be determined in a sample from normal or healthy tissue (of the same origin as the sample used in step a)) from the same patient

In another embodiment of the invention, a method of selecting a composition for inhibiting the progression of cancer in a patient is provided, the method comprising:
a) providing a sample comprising cancer cells from the patient;
b) separately exposing aliquots of the sample in the presence of a plurality of test compositions;
c) comparing the level of expression of the polypeptide according to the invention or a peptide fragment according to the invention in each of the aliquots; and
d) selecting one of the test compositions which alters the level of expression of the polypeptide in the aliquot containing that test composition, relative to other test compositions.

In both methods, the sample is a tissue sample, blood or blood derived cells, primary cell cultures from patients, stool, lymph or a tissue-associated fluid or urine.

The presence of said polypeptide or peptide fragment is preferably detected using a reagent which specifically binds with said polypeptide or peptide fragment, preferably the reagent is selected from the group consisting of an antibody, an antibody derivative, and an antibody fragment. This is described in more detail below.

In another preferred embodiment, a method of selecting a composition for inhibiting the progression of cancer in a patient is provided, the method comprising:
a) separately exposing aliquots of a sample comprising cancer cells from the patient in the presence of a plurality of test compositions;
b) comparing the level of expression of the polypeptide according to the invention or a peptide fragment according to the invention in each of the aliquots; and
c) selecting one of the test compositions which alters the level of expression of the polypeptide in the aliquot containing that test composition, relative to other test compositions.

Preferably, the antibody is an antibody according to the invention. Preferably, the human cancer cell is a breast cancer cell, a colorectal cancer cell or a leukemia cell, preferably a Philadelphia chromosome (or Bcr-Abl) positive leukemia cell, more preferably a chronic myeloid leukemia cell or an acute lymphoblastic leukemia cell. Preferably, the sample is a tissue sample, blood or blood derived cells, primary cell cultures from patients, stool, lymph or a tissue-associated fluid or urine.

In another preferred embodiment, a method to predict which patients will respond to a tyrosine kinase inhibitor drug in patients with disorders whose underlying pathology involves the discontrol of a tyrosine kinase is provided comprising:
a) contacting a sample from a patient with an antibody according to the invention,
b) determining the level of phosphorylated polypeptide bound by the antibody of step a), and
c) comparing the level of phosphorylated polypeptide determined in step (b) for the sample with the level of phosphorylated protein in a reference sample, thereby detecting the responsiveness to a tyrosine kinase inhibitor drug in patients with disorders whose underlying pathology involves the discontrol of a tyrosine kinase.

Preferably, the disorder is a hyperproliferative disease, more preferably cancer and preferably the patients are cancer patients, more preferably the disorder is Philadelphia chromosome positive leukaemia and the patients are Philadelphia chromosome positive leukemia patients (WO03/087404). More preferably, the disorder is chronic myeloid leukaemia abbreviated as CML or (Bcr-Abl positive) acute lymphoblastic leukemia abreviated as ALL and the patients are chronic myeloid leukaemia or acute lymphoblastic leukemia patients. Preferably, the reference sample is a sample from a patient who responds to the tyrosine kinase inhibitor drug, preferably from a chronic myeloid leukaemia patient who responds to the tyrosine kinase inhibitor drug. Preferably, the tyrosine kinase inhibitor is imatinib mesylate (Imatinib, GLEEVEC®, GLIVEC® or S11571) which is a drug used for the treatment of disorders whose underlying pathology involves the discontrol of a tyrosine kinase, preferably chronic myeloid leukaemia (Druker et al., M Engl. J. Med., Vol. 344, No. 14, pp. 1031-1037 (2001); WO03/087404). Imatimib mesylate is designated chemically as 4-[(4-Methyl-1-piperazinyl)methyl]-N-[4-methyl-3-[[4-(3-pyridinyl)-2-pyrimidinyl] amino]-phenyl] benzamide methane sulfonate. The phosphorylated polypeptide is preferably a polypeptide according to the invention or a peptide fragment according to the invention.

In a preferred embodiment, the polypeptide according to the invention or the peptide fragment according to the invention is used for the determination of the potential of a human cancer cell for tumor progression or for the prediction which patient with a disorder whose underlying pathology involves the discontrol of a tyrosine kinase will respond to a tyrosine kinase inhibitor drug, preferably imnatinib mesylate (Imatinib or GLEEVECQ) or GLIVEC or STI571). Preferably, the disorder whose underlying pathology involves the discontrol of a tyrosine kinase is a disorder as described above.

In an alternative embodiment the ratio of phosphorylated versus unphosphorylated polypeptide is determined for each sample.

An exemplary method for detecting the presence or absence of a phosphorylated polypeptide according to the invention in a biological sample involves obtaining a biological sample (e.g. a biopsy) from a test subject and contacting the biological sample with a compound or an agent capable of detecting the polypeptide. The detection methods of the invention can thus be used to detect protein for example in a biological sample in vitro as well as in vivo. In vitro techniques for detection of a polypeptide according to the invention include enzyme linked immunosorbent assays (ELISAs), Western blots, immunoprecipitations, immunohistochemistry and immunofluorescence. Furthermore in vivo techniques for detection of a polypeptide according to the invention include introducing into a subject a labelled antibody directed against the polypeptide. For example the antibody can be labelled with a radioactive marker whose presence and location in a subject can be detected by standard imaging techniques.

A general principle of such diagnostic and prognostic assays involves preparing a sample or reaction mixture that may contain a protein or polynucleotide and a probe, under appropriate conditions and for a time sufficient to allow the protein or nucleotide and probe to interact and bind, thus forming a complex that can be removed and/or detected in the reaction mixture. These assays can be conducted in a variety of ways.

For example one method to conduct such an assay would involve anchoring the protein or nucleotide on the one hand or probe on the other onto a solid phase supports also referred to as a substrate and detecting complexes comprising the target nucleic acid molecule or protein and the probe anchored on the solid phase at the end of the reaction. In one embodiment of such a method a sample from a subject which is to be assayed for presence and/or concentration of the proteins or nucleotides encoded by the nucleic acid molecule can be anchored onto a carrier or solid phase support. In another embodiment the reverse situation is possible in which the probe can be anchored to a solid phase and a sample from a subject can be allowed to react as an unanchored component of the assay.

There are many established methods for anchoring assay components to a solid phase. These include without limitation the protein or nucleic acid molecules according to the invention or probe molecules which are immobilized through conjugation of biotin and streptavidin. Such biotinylated assay components can be prepared from biotin-NHS (N-hydroxy-succininide) using techniques known in the art (e.g. biotinylation kit, Pierce Chemicals, Rockford, Ill.) and immobilized in the wells of streptavidin-coated 96 well plates (Pierce Chemical). In certain embodiments the surfaces with immobilized assay components can be prepared in advance and stored.

Other suitable carriers or solid phase supports for such assays include any material capable of binding the class of molecule to which the protein according to the invention or nucleotide or probe belongs. Well-known supports or carriers include but are not limited to glass, polystyrene, nylon, polypropylene, nylon, polyethylene, dextran, amylases, natural and modified celluloses, polyacrylamides, gabbros and magnetite.

In order to conduct assays with the above-mentioned approaches the non immobilized component is added to the solid phase upon which the second component is anchored. After the reaction is complete uncomplexed components may be removed (e.g. by washing) under conditions such that any complexes formed will remain immobilized upon the solid phase. The detection of complexes comprising the protein of the invention and the probe anchored to the solid phase can be accomplished in a number of methods outlined herein. In a preferred embodiment the probe when it is the unanchored assay component can be labelled for the purpose of detection and readout of the assay, either directly or indirectly with detectable labels discussed herein and which are well-known to one skilled in the art.

It is also possible to directly detect complexes comprising a protein of the invention and the probe without further manipulation or labelling of either component (the protein of the invention or nucleotide or the probe) for example by utilizing the technique of fluorescence energy transfer (see for example U.S. Pat. No. 5,631,169 and U.S. Pat. No. 4,868,103). A fluorophore label on the first ‘donor’ molecule is selected such that upon excitation with incident light of appropriate wavelength its emitted fluorescent energy will be absorbed by a fluorescent label on a second ‘acceptor’ molecule, which in turn is able to fluoresce due to the absorbed energy. Alternately the ‘donor’ protein molecule may simply utilize the natural fluorescent energy of tryptophan residues. Labels are chosen that emit different wavelengths of light such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. A PET binding event can be conveniently measured through standard fluorometric detection means well known in the art (e.g. using a fluorimeter).

In another embodiment determination of the ability of a probe to recognize a protein can be accomplished without labelling either assay component (probe) by utilizing a technology such as real time Biomolecular Interaction Analysis (BIA) (see e.g. Sjolander, S. and Urbaniczky, C., Anal. Chem. 63 (1991) 2338-2345 and Szabo, A., et al., Curr. Opin. Struct. Biol. 5 (1995) 699-705). As used herein “BIA” or “surface plasmon resonance” is a technology for studying biospecific interactions in real-time without labelling any of the interactants (e.g. BIAcore). Changes in the mass at the binding surface (indicative of a binding event) result in alterations of the refractive index of light near the surface (the optical phenomenon of surface plasmon resonance (SPR)) resulting in a detectable which can be used as an indication of real-time reactions between biological molecules.

Alternatively in another embodiment, analogous diagnostic and prognostic assays can be conducted with the protein of the invention and the probe as solutes in a liquid phase. In such an assay complexes comprising the protein of the invention and the probe are separated from uncomplexed components by any of a number of standard techniques, including but not limited to differential centrifugation, chromatography, electrophoresis and immunoprecipitation. In differential centrifugation such complexes may be separated from uncomplexed assay components through a series of centrifugal steps due to the different sedimentation equilibria of complexes based on their different sizes and densities (see for example Rivas, G., and Minton, A. P., Trends Biochem Sci. 18 (1993) 284-287). Standard chromatographic techniques may also be utilized to separate such complexes from uncomplexed components. For example gel filtration chromatography separates molecules based on size and through the utilization of an appropriate gel filtration resin in a column format for example the relatively larger complexes may be separated from the relatively smaller uncomplexed components. Similarly the different charge properties of such complexes as compared to the uncomplexed components may be exploited to differentiate the complexes from uncomplexed components for example through the utilization of ion-exchange chromatography resins. Such resins and chromatographic techniques are well known to a person skilled in the art (see e.g. Heegaard, N. H., J. Mol. Recognit. 11 (1998) 141-148; Hage, D. S., and Tweed, S. A., J. Chromatogr. B. Biomed. Sci. Appl. 699 (1997) 499-525). Gel electrophoresis may also be employed to separate such complexes from unbound components (see e.g., Ausubel et al., ed., Current Protocols in Molecular Biology, John Wiley & Sons, New York, 1987 1999). In this technique protein complexes are separated based on size or charge for example. In order to maintain the binding interaction during the electrophoretic process non-denaturing gel matrix materials and conditions in the absence of reducing agent are typically preferred. Appropriate conditions to the particular assay and components thereof will be well known to one skilled in the art.

In another embodiment of the present invention a polypeptide of the invention is detected. A preferred agent for detecting a polypeptide of the invention is an antibody capable of binding to a polypeptide according to the invention preferably an antibody with a detectable label. Antibodies can be polyclonal or more preferably monoclonal. An intact antibody or a fragment thereof (e.g. Fab or F(ab′)₂) can be used. The term “labelled” with regard to the probe or antibody is intended to encompass direct labelling of the probe or antibody by coupling (i. e. physically linking) a detectable substance to the probe or antibody as well as indirect labelling of the probe or antibody by reactivity with another reagent that is directly labelled. Examples of indirect labelling include detection of a primary antibody using a fluorescently labelled secondary antibody and end-labelling of a DNA probe with biotin such that it can be detected with fluorescently labelled streptavidin.

Proteins from cells can be isolated using techniques that are well-known to a person skilled in the art. The protein isolation methods employed can for example be such as those described in Harlow and Lane, Antibodies: Laboratory Manual, Cold Spring Harbor Laboratory Press, Cold Spring Harbor, N.Y., 1988). A variety of formats can be employed to determine whether a sample contains a protein that binds to a given antibody. Examples of such formats include, but are not limited to, enzyme immunoassay (EIA), radioimmunoassay (RIA), Western blot analysis, immunohisto-chemistry and enzyme linked immunoabsorbent assay (ELISA).

A skilled artisan can readily adapt known protein/antibody detection methods for use in determining whether cancer cells express a nucleic acid molecule of the present invention and modifies the expressed protein.

In one format antibodies or antibody fragments can be used in methods such as Western blots, immunohistochemistry or immunofluorescence techniques to detect the expressed proteins. In such uses it is generally preferable to immobilize either the antibody, proteins or cells containing proteins on a solid support. Well-known supports or carriers include glass, polystyrene, polypropylene, polyethylene, dextran, nylon, amylases, natural and modified celluloses, polyacrylamides, gabbros and magnetite.

One skilled in the art will know many other suitable carriers for binding antibody or antigen and will be able to adapt such support for use with the present invention. For example protein isolated from cancer cells can be run on a polyacrylamide gel electrophoresis and immobilized onto a solid phase support such as nitrocellulose or PVDF. The support can then be washed with suitable buffers followed by treatment with the detectably labelled antibody. The solid phase support can then be washed with the buffer a second time to remove unbound antibody. The amount of bound label on the solid support can then be detected by conventional means.

The invention also encompasses kits for detecting the presence of a polypeptide according to the invention in a biological sample (e.g. a tissue-associated body fluid). Such kits can be used to determine if a subject is suffering from or is at increased risk of developing metastatic cancer. For example the kit can comprise a labelled compound or agent capable of detecting a polypeptide or an mRNA encoding a polypeptide according to the invention in a biological sample and means for determining the amount of the polypeptide or mRNA in the sample (e.g. an antibody which binds the polypeptide or an oligonucleotide probe which binds to DNA or mRNA encoding the polypeptide). Kits can also include instructions for interpreting the results obtained using the kit.

Therefore, in an embodiment of the invention a kit for the detection or determination of the amount of the polypeptide according to the invention or the peptide fragment according to the invention in a biological sample is provided which comprises:
(a) an antibody according to the invention and
(b) a label for qualitatively or quantitatively detecting an immunoconjugate of the antibody and the polypeptide or the peptide fragment. This is described in more detail below.

For antibody-based kits the kit can comprise for example: (1) a first antibody (e.g. attached to a solid support) which binds to a polypeptide according to the invention and optionally (2) a second different antibody which binds to either the polypeptide or the first antibody and is conjugated to a detectable label. For oligonucleotide-based kits the kit can comprise for example (1) an oligonucleotide e.g. a detectably labelled oligonucleotide which hybridizes to a nucleic acid sequence encoding a polypeptide according to the invention or (2) a pair of primers useful for amplifying the nucleic acid molecule according to the invention. The kit can also comprise e.g. a buffering agent, a preservative or a protein stabilizing agent The kit can further comprise components necessary for detecting the detectable label (e.g. an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

V. Pharmaceutical Compositions

In a preferred embodiment of the invention, agents which modulate expression or activity of a polypeptide according to the invention are provided for use in medicine, i.e. a polypeptide or a peptide fragment according to the invention, a non-phosphorylable polypeptide according to the invention, a non-phosphorylable peptide fragment according to the invention, a nucleic acid molecule according to the invention or an antibody according to the invention is provided for use in medicine. Further, a pharmaceutical composition comprising polypeptide or a peptide fragment according to the invention, a non-phosphorylable polypeptide according to the invention, a non-phosphorylable peptide fragment according to the invention, a nucleic acid molecule according to the invention or an antibody according to the invention together with a pharmaceutically acceptable carrier is provided. In another embodiment, the polypeptide or the peptide fragment according to the invention, the non-phosphorylable polypeptide according to the invention, the non-phosphorylable peptide fragment according to the invention, the nucleic acid molecule according to the invention or the antibody according to the invention is used for the preparation of a pharmaceutical composition for the treatment of hyperproliferative disease, preferably cancer.

In another, substances may be used in medicine that inhibit the tyrosine modification of p27.

The invention includes methods for preparing pharmaceutical compositions for modulating the expression or activity of a polypeptide according to the invention. Such methods comprise formulating a pharmaceutically acceptable carrier with an agent which modulates expression or activity of a polypeptide or nucleic acid according to the invention. Such compositions can further include additional active agents. Methods for making such formulations or compositions can be found in manuals, e.g. Remington Pharmaceutical Science, 18th Ed., Merck Publishing Co. Eastern Pa., USA (1990). Thus the invention further includes methods for preparing a pharmaceutical composition by formulating a pharmaceutically acceptable carrier with an agent which modulates expression or activity of a polypeptide according to the invention and one or more additional active compounds. As used herein the language “pharmaceutically acceptable carrier” is intended to include any and all solvents, dispersion media, coatings, antibacterial and antifungal agents, isotonic and absorption delaying agents and the like compatible with pharmaceutical administration. The use of such media and agents for pharmaceutically active substances is well known in the art except insofar as any conventional media or agent is incompatible with the active compound use thereof in the compositions is contemplated. Supplementary active compounds can also be incorporated into the compositions.

The invention also provides methods (also referred to herein as “screening assays”) for identifying modulators, i. e. candidate or test compounds or agents (e.g. peptides, peptidomimetics, peptoids, small molecules or other drugs) which (a) bind to the polypeptide according to the invention or (b) have a modulatory (e.g. stimulatory or inhibitory) effect on the activity of the nucleic acid molecule or more specifically (c) have a modulatory effect on the interactions of a protein with one or more of its natural substrates (e.g. peptide, protein, hormone, co-factor or nucleic acid). Such assays typically comprise a reaction between the protein of the invention and one or more assay components. The other components may be either the test compound itself or a combination of test compound and a natural binding partner of the protein according to the invention.

Therefore, a method of deriving a candidate agent or test compound is provided, said method comprising:
(a) contacting a sample containing cancer cells, with said candidate agent or test compound;
(b) determining the level of expression of the polypeptide according to the invention or a peptide fragment according to the invention in the sample contacted with the candidate agent or test compound and determining the level of expression of the polypeptide in a sample not contacted with the candidate agent or test compound;
(c) observing the effect of the candidate agent or test compound by comparing the level of expression of the polypeptide or the peptide fragment in the sample contacted with the candidate agent or test compound and the level of the polypeptide or the peptide fragment in the sample not contacted with the candidate agent or test compound,
(d) deriving said agent from said observed effect,
   wherein an at least 1.5 fold difference or a less than 0.75 fold difference between the level of expression of the polypeptide or the peptide fragment in the sample contacted with the candidate agent or test compound and the level of expression of the polypeptide or the peptide fragment in the sample not contacted with the candidate agent or test compound is an indication of an effect of the candidate agent or test compound. Preferably, said candidate agent or test compound is a candidate inhibitory agent or a candidate enhancing agent.

The test compounds of the present invention may be obtained from any available source including systematic libraries of natural and/or synthetic compounds. Test compounds may also be obtained by any of the numerous approaches in combinatorial library methods known in the art, including biological libraries, peptoid libraries (libraries of molecules having the functionalities of peptides but with a novel non peptide backbone which are resistant to enzymatic degradation but which nevertheless remain bioactive, see e.g. Zuckermann, R. N., et al., J. Med. Chem. 37 (1994) 2678-2685), spatially addressable parallel solid phase or solution phase libraries, synthetic library methods requiring deconvolution, the ‘one-bead one-compound’ library method and synthetic library methods using affinity chromatography selection. The biological library and peptoid library approaches are limited to peptide libraries while the other four approaches are applicable to peptide, non-peptide oligomer or small molecule libraries of compounds (Lam, K. S., Anticancer Drug Des. 12 (1997) 145-167).

Examples of methods for the synthesis of molecular libraries can be found in the art for example in DeWitt, S. H., et al., Proc. Natl. Acad. Sci. USA 90 (1993) 6909-6913; Erb, E., et al., Proc. Natl. Acad. Sci. USA 91 (1994) 11422-11426; Zuckermann, R. N., et al., J. Med Chem. 37 (1994) 2678-2685; Cho, C. Y., et al., Science 261 (1993) 1303-1305; Carrell et al., Angew. Chem. Int. Ed. Erg. 33 (1994) 2059; Carell et al., Angew. Chem. Int. Ed. Engl. 33 (1994) 2061 and in Gallop, M. A., et al., J. Med. Chem. 37 (1994) 1233-1251.

Libraries of compounds may be presented in solution (e.g. Houghten, R. A., et al., Biotechniques 13 (1992) 412-421) or on beads (Lam, K. S., et al., Nature 354 (1991) 82-84), chips (Fodor, S. P., et al., Nature 364 (1993) 555-556), bacteria and/or spores, (U.S. Pat. No. 5,223,409), plasmids (Cull, M. G., et al., Proc. Natl. Acad. Sci. USA 89 (1992) 1865-1869) or on phage (Scott, J. K, and Smith, G. P., Science 249 (1990) 386-390; Devlin, J. J., et al., Science 249 (1990) 404-406; Cwirla, S. E., et al, Proc. Natl. Acad. Sci. 87 (1990) 6378-6382; Felici, F., et al., Mol. Biol. 222 (1991) 301-310; U.S. Pat. No. 5,223,409). In one embodiment the invention provides assays for screening candidate or test compounds which are substrates of the protein of the invention or biologically active portion thereof. In another embodiment the invention provides assays for screening candidate or test compounds which bind to a protein according to the invention or biologically active portion thereof.

Determining the ability of the test compound to directly bind to a polypeptide of the invention can for example be accomplished by coupling the compound with a radioisotope or enzymatic label such that binding of the compound to the protein can be determined by detecting the protein compound in a labelled complex. For example compounds (e.g. substrates of the protein of the invention) can be labelled with 25I, 32P, 35S, 14C or 3H either directly or indirectly and the radioisotope detected by direct counting of radioemission or by scintillation counting. Alternatively assay components can be enzymatically labelled with for example horseradish peroxidase, alkaline phosphatase or luciferase and the enzymatic label detected by determination of conversion of an appropriate s substrate to product.

In another embodiment the invention provides assays for screening candidate or test compounds which modulate the activity of the polypeptide of the invention or a biologically active portion thereof. In all likelihood the protein can in vivo interact with one or more molecules such as but not limited to peptides, proteins, hormones, cofactors and nucleic acids. For the purposes of this discussion such cellular and extracellular molecules are referred to herein as “binding partners” or protein “substrate”.

One necessary embodiment of the invention in order to facilitate such screening is the use of the protein according to the invention to identify its natural in vivo binding partners. There are many ways to accomplish this which are known to a person skilled in the art One example is the use of the protein of the invention as “bait protein” in a two-hybrid assay or three-hybrid assay (see e.g. U.S. Pat. No. 5,283,317; Zervos, A. S., et al., Cell 72 (1993) 223-232; Madura, K, et al., J. Biol. Chem. 268 (1993) 12046-12054; Bartel, P., et al., Biotechniques 14 (1993) 920-924; Iwabuchi, K, et al., Oncogene 8 (1993) 1693-1696; WO 94/10300) in order to identify other proteins which bind to or interact with the protein (binding partners) and therefore are possibly involved in the natural function of the protein. Such protein binding partners are also likely to be involved in the propagation of signals by the protein or downstream elements of a gene-mediated signalling pathway. Alternatively such protein binding partners may also be found to be inhibitors of the protein.

The two-hybrid system is based on the modular nature of most transcription factors which consist of separable DNA-binding and activation domains. Briefly the assay utilizes two different DNA constructs. In one construct the gene that encodes a protein fused to a gene encoding the DNA binding domain of a known transcription factor (e.g. GAL-4). In the other construct a DNA sequence from a library of DNA sequences that encodes an unidentified protein (“prey” or “sample”) is fused to a gene that codes for the activation domain of the known transcription factor. If the “bait” and the “prey” proteins are able to interact in vivo forming a gene dependent complex, the DNA-binding and activation domains of the transcription factor are brought into dose proximity. This proximity allows transcription of a reporter gene (e.g. LacZ) which is operably linked to a transcriptional regulatory site responsive to the transcription factor. Expression of the reporter gene can be readily detected and cell clones containing the functional transcription factor can be isolated and used to obtain the cloned gene which encodes the protein which interacts with the protein of the invention.

In a further embodiment assays may be devised through the use of the invention for the purpose of identifying compounds which modulate (e.g. affect either positively or negatively) interactions between the protein of the invention and its substrates and/or binding partners. Such compounds can include, but are not limited to, molecules such as antibodies, peptides, hormones, oligonucleotides, nucleic acids and analogues thereof.

Such compounds may also be obtained from any available source, including systematic libraries of natural and/or synthetic compounds. The preferred assay components for use in this embodiment is the protein of the invention, the known binding partner and/or substrate of same and the test compound. Test compounds can be supplied from any source.

The basic principle of the assay systems used to identify compounds that interfere with the interaction between the protein and its binding partner involves preparing a reaction mixture containing the protein and its binding partner under conditions and for a time sufficient to allow the two products to interact and bind thus forming a complex In order to test an agent for inhibitory activity, the reaction mixture is prepared in the presence and absence of the test compound. The test compound can be initially included in the reaction mixture or can be added at a time subsequent to the addition of the protein and its binding partner. Control reaction mixtures are incubated without the test compound or with a placebo. The formation of any complexes between the protein and its binding partner is then detected. The formation of a complex in the control reaction but less or no such formation in the reaction mixture containing the test compound indicates that the compound interferes with the interaction of the protein of the invention and its binding partner. Conversely the formation of more complex in the presence of compound than in the control reaction indicates that the compound may enhance interaction of the protein and its binding partner.

The assay for compounds that interfere with the interaction of the protein with its binding partner may be conducted in a heterogeneous or homogeneous format.

Heterogeneous assays involve anchoring either the protein of the invention or its binding partner onto a solid phase and detecting complexes anchored to the solid phase at the end of the reaction. In homogeneous assays the entire reaction is carried out in a liquid phase. In either approach the order of addition of reactants can be varied to obtain different information about the compounds being tested. For example test compounds that interfere with the interaction between the protein of the invention and the binding partners (e.g. by competition) can be identified by conducting the reaction in the presence of the test substance, i. e. by adding the test substance to the reaction mixture prior to or simultaneously with the protein and its interactive binding partner.

Alternatively test compounds that disrupt preformed complexes e.g. compounds with higher binding constants that displace one of the components from the complex can be tested by adding the test compound to the reaction mixture after complexes have been formed. The various formats are briefly described below.

In a heterogeneous assay system either the protein of the invention or its binding partner is anchored onto a solid surface or matrix while the other corresponding non-anchored component may be labelled, either directly or indirectly. In practice nicrotitre plates are often utilized for this approach. The anchored species can be immobilized by a number of methods either non-covalent or covalent that are typically well known to one who practices the art. Non-covalent attachment can often be accomplished simply by coating the solid surface with a solution of the protein or its binding partner and drying.

Alternatively an immobilized antibody specific for the assay component to be anchored can be used for this purpose. Such surfaces can often be prepared in advance and stored.

In related embodiments a fusion protein can be provided which adds a domain that allows one or both of the assay components to be anchored to a matrix. For example glutathione-S-transferase/protein of the invention fusion proteins or glutathione-S-transferase/binding partner can be adsorbed onto glutathione Sepharose beads (Sigma Chemical, St. Louis, Mo.) or glutathione derivatized microtitre plates which are then combined with the test compound or the test compound and either the non-adsorbed protein or its binding partner and the mixture incubated under conditions conducive to complex formation (e.g. physiological conditions). Following incubation the beads or microtitre plate wells are washed to remove any unbound assay components, the immobilized complex assessed either directly or indirectly for example as described above. Alternatively the complexes can be dissociated from the matrix and the level of protein binding or activity determined using standard techniques. Other techniques for immobilizing proteins on matrices can also be used in the screening assays of the invention. For example either a protein of the invention or its binding partner can be immobilized utilizing conjugation of biotin and streptavidin. Biotinylated protein or target molecules can be prepared from biotin-NHS (N-hydroxy succinimide) using techniques known in the art (e.g. biotinylation kit, Pierce Chemicals, Rockford, Ill.) and immobilized in the wells of streptavidin-coated 96 well plates (Pierce Chemical). In certain embodiments the protein-immobilized surfaces can be prepared in as advance and stored.

In order to conduct the assay the corresponding partner of the immobilized assay component is exposed to the coated surface with or without the test compound. After the reaction is complete unreacted assay components are removed (e.g. by washing) and any complexes formed will remain immobilized on the solid surface. The detection of complexes anchored on the solid surface can be accomplished in a number of ways.

Where the non-immobilized component is pre-labelled, the detection of label immobilized on the surface indicates that complexes were formed. Where the non-immobilized component is not pre-labelled, an indirect label can be used to detect complexes anchored on the surface e.g. using a labelled antibody specific for the initially non-immobilized species (the antibody in turn can be directly labelled or indirectly labelled). Depending upon the order of addition of reaction components test compounds which modulate (inhibit or enhance) complex formation or which disrupt preformed complexes can be detected.

In an alternate embodiment of the invention a homogeneous assay may be used. This is typically a reaction analogous to those mentioned above which is conducted in a liquid phase in the presence or absence of the test compound. The formed complexes are then separated from unreacted components and the amount of complex formed is determined. As mentioned for heterogeneous assay systems the order of addition of reactants to the liquid phase can yield information about which test compounds modulate (inhibit or enhance) complex formation and which disrupt preformed complexes. In such a homogeneous assay the reaction products may be separated from unreacted assay components by any of a number of standard techniques including but not limited to differential centrifugation, chromatography, electro-phoresis and immunoprecipitation. In differential centrifugation complexes of molecules may be separated from uncomplexed molecules through a series of centrifugal steps due to the different sedimentation equilibria of complexes based on their different sizes and densities (see for example Rivas, G., and Minton, A. P., Trends Biochem. Sci. 18 (1993) 284-287). Standard chromatographic techniques may also be utilized to separate complexed molecules from uncomplexed ones. For example gel filtration chromatography separates molecules based on size and through the utilization of an appropriate gel filtration resin in a column format for example the relatively larger complex may be separated from the relatively smaller uncomplexed components.

Similarly the relatively different charge properties of the complex as compared to the uncomplexed molecules may be exploited to differentially separate the complex from the remaining individual reactants for example through the use of ion-exchange chromatography resins. Such resins and chromatographic techniques are well known to a person skilled in the art (see e.g. Heegaard, N. H., J. Mol. Recognit. 11 (1998) 141-148; Hage, D. S., and Tweed, S A, J. Chromatogr. B. Biomed. Sci Appl., 699 (1997) 499-525). Gel electrophoresis may also be employed to separate complexed molecules from unbound species (see e.g. Ausubel et al. (eds.); In: Current Protocols in Molecular Biology, J. Wiley & Sons, New York, 1999). In this technique protein or nucleic acid complexes are separated based on size or charge for example. In order to maintain the binding interaction during the electrophoretic process non-denaturing gels in the absence of reducing agent are typically preferred but conditions appropriate to the particular interactants will be well known to a person skilled in the art Immunoprecipitation is another common technique utilized for the isolation of a protein-protein complex from solution (see e.g. Ausubel et al., supra). In this technique all proteins binding to an antibody specific to one of the binding molecules are precipitated from solution by conjugating the antibody to a polymer bead that may be readily collected by centrifugation. The bound assay components are released from the beads (through a specific proteolysis event or other technique well-known in the art which will not disturb the protein-protein interaction in the complex) and a second immunoprecipitation step is performed, this time utilizing antibodies specific for the correspondingly different interacting assay component. In this manner only formed complexes should remain attached to the beads. Variations in no complex formation in both the presence and the absence of a test compound can be compared thus offering information about the ability of the compound to modulate interactions between the protein of the invention and its binding partner.

Also within the scope of the present invention are methods for direct detection of interactions between the protein of the invention and its natural binding partner and/or a test compound in a homogeneous or heterogeneous assay system without further sample manipulation. For example the technique of fluorescence energy transfer may be utilized (see e.g. U.S. Pat. No. 5,631,169; U.S. Pat. No. 4,868,103). Generally this technique involves the addition of a fluorophore label on a first ‘donor’ molecule (edge, test compound) such that its emitted fluorescent energy will be absorbed by a fluorescent label on a second, ‘acceptor’ molecule (e.g. test compound), which in turn is able to fluoresce due to the absorbed energy.

Alternately the ‘donor’ protein molecule may simply utilize the natural fluorescent energy of tryptophan residues. Labels are chosen that emit different wavelengths of light such that the ‘acceptor’ molecule label may be differentiated from that of the ‘donor’. Since the efficiency of energy transfer between the labels is related to the distance separating the molecules spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well-known in the art (e.g. using a fluorimeter). A test substance which either enhances or hinders participation of one of the species in the preformed complex will result in the generation of a signal variant to that of background. In this way test substances that modulate interactions between a protein of the invention and its binding partner can be identified in controlled assays.

In another embodiment modulators of nucleic acid molecule expression are identified in a method wherein a cell is contacted with a candidate compound and the expression of mRNA or protein encoded by a nucleic acid molecule is determined. The level of expression of mRNA or protein in the presence of the candidate compound is compared to the level of expression of mRNA or protein in the absence of the candidate compound. The candidate compound can then be identified as a modulator of nucleic acid molecule expression based on this comparison. For example when expression of nucleic acid molecule or protein is greater (statistically significantly greater) in the presence of the candidate compound than in its absence the candidate compound is identified as a stimulator of nucleic acid molecule expression. Conversely when expression of nucleic acid molecule mRNA or protein is less (statistically significantly less) in the presence of the candidate compound than in its absence, the candidate compound is identified as an inhibitor of nucleic acid molecule expression. The level of nucleic acid molecule expression in the cells can be determined by methods described herein for detecting nucleic acid molecule mRNA or protein.

In another aspect the invention pertains to a combination of two or more of the assays described herein. For example a modulating agent can be identified using a cell-based or a cell free assay and the ability of the agent to modulate the activity of a protein of the invention can be further confirmed in viva, e.g., in a whole animal model for cellular transformation and/or tumorigenesis.

This invention further pertains to novel agents identified by the above-mentioned screening assays. Accordingly it is within the scope of this invention to further use an agent identified as described herein in an appropriate animal model. For example an agent identified as described herein (e.g. a gene or protein modulating agent, an antisense gene nucleic acid molecule, a protein specific antibody or a protein binding partner according to the invention) can be used in an animal model to determine the efficacy, toxicity or side effects of treatment with such an agent.

Alternatively an agent identified as described herein can be used in an animal model to determine the mechanism of action of such an agent Furthermore this invention pertains to uses of novel agents identified by the above-described screening assays for treatments as described herein.

It is understood that appropriate doses of small molecule agents and protein or polypeptide agents depends upon a number of factors within the knowledge of the ordinarily skilled physician, veterinarian or researcher. The dose(s) of these agents will vary for example depending upon the identity, size and condition of the subject or sample being treated, further depending upon the route by which the composition is to be administered if applicable and the effect which the practitioner desires the agent to have upon the nucleic acid or polypeptide of the invention. Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram or about 1 microgram per kilogram to about 50 micrograms per kilogram). Exemplary doses of a protein or polypeptide include gram, milligram or microgram amounts per kilogram of subject or sample weight (e.g. about 1 microgram per kilogram to about 5 grams per kilogram about 100 micrograms per kilogram to about 500 milligrams per kilogram or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention a physician, veterinarian or researcher can for example prescribe a relatively low dose at first subsequently increasing the dose until an appropriate response is obtained. In addition it is understood that the specific dose level for any particular animal subject will depend upon a variety of factors including the activity of the specific agent employed, the age, body weight, general health, gender and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral e.g. intravenous, intradermal, subcutaneous, oral (e.g. inhalation), transdermal (topical), transmucosal and rectal administration. Solutions or suspensions used for parenteral, intradermal or subcutaneous application can include the following components a sterile diluent such as water for injection, saline solution, fixed oils, polyethylene glycols, glycerine, propylene glycol or other synthetic solvents, antibacterial agents such as benzyl alcohol or methyl parabens, antioxidants such as ascorbic acid or sodium bisulfite, chelating agents such as ethylenediamine-tetraacetic acid, buffers such as acetates, citrates or phosphates and agents for the adjustment of tonicity such as sodium chloride or dextrose. pH can be adjusted with acids or bases such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration suitable carriers include physiological saline, bacteriostatic water, Cremophor EL (BASF; Parsippany, N.J.) or phosphate buffered saline (PBS). In all cases the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing for example water ethanol, polyol (for example glycerol, propylene glycol and liquid polyethylene glycol and the like) and suitable mixtures thereof. The proper fluidity can for example be maintained by the use of a coating such as lecithin, by the maintenance of the required particle size in the case of dispersion and by the use of surfactants.

Prevention of the action of microorganisms can be achieved by various antibacterial and antifungal agents for example parabens, chlorobutanol, phenol, ascorbic acid, thimerosal and the like. In many cases it will be preferable to include isotonic agents for example sugars, polyalcohols such as mannitol, sorbitol or sodium chloride in the composition. Prolonged absorption of the injectable compositions can be brought about by including in the composition an agent which delays absorption for example aluminium monostearate and gelatin.

Sterile injectable solutions can be prepared by incorporating the active compound (e.g. a polypeptide or antibody) in the required amount in an appropriate solvent with one or a combination of ingredients enumerated above as required followed by filtered sterilization. Generally dispersions are prepared by incorporating the active compound into a sterile vehicle which contains a basic dispersion medium and then incorporating the required other ingredients from those enumerated above. In the case of sterile powders for the preparation of sterile injectable solutions the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches or capsules. Oral compositions can also be prepared using a fluid carrier for use as a mouthwash, wherein the compound in the fluid carrier is applied orally and swished and expectorated or swallowed.

Pharmaceutically compatible binding agents and/or adjuvant materials can be included as part of the composition. The tablets, pills, capsules, troches and the like can contain any of the following ingredients or compounds of a similar nature. A binder such as microcrystalline cellulose, gum tragacanth or gelatin, an excipient such as starch or lactose, a disintegrating agent such as alginic acid, Primogel or corn starch, a lubricant such as magnesium stearate or Sterotes, a glidant such as colloidal silicon dioxide, a sweetening agent such as sucrose or saccharin or a flavouring agent such as peppermint, methyl salicylate or orange flavouring.

For administration by inhalation the compounds are delivered in the form of an aerosol spray from a pressurized container or dispenser which contains a suitable propellant, e.g. a gas such as carbon dioxide or a nebulizer.

Systemic administration can also be by transmucosal or transdermal means. For transmucosal or transdermal administration, penetrants appropriate to the barrier to be permeated are used in the formulation. Such penetrants are generally known in the art and include for example for transmucosal administration, detergents, bile salts and fusidic acid derivatives. Transmucosal administration can be accomplished through the use of nasal sprays or suppositories. For transdermal administration the active compounds are formulated into ointments, salves, gels, or creams as generally known in the art.

The compounds can also be prepared in the form of suppositories (e.g. with conventional suppository bases such as cocoa butter and other glycerides) or retention enemas for rectal delivery.

In one embodiment the active compounds are prepared with carriers that will protect the compound against rapid elimination from the body such as a controlled release formulation including implants and microencapsulated delivery systems.

Biodegradable biocompatible polymers can be used such as ethylene vinyl acetate, polyanhydrides, polyglycolic acid, collagen, polyorthoesters and polylactic acid. Methods for preparation of such formulations will be apparent to those skilled in the art.

The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to persons skilled in the art for example as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved and the limitations inherent in the art of compounding such an active compound for the treatment of individuals.

For antibodies the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally partially human antibodies and fully human antibodies have a longer half-life within the human body than other antibodies. Accordingly lower dosages and less frequent administration is often possible. Modifications such as lipidation can be used to stabilize antibodies and to enhance uptake and tissue penetration. A method for lipidation of antibodies is described by Cruikshank, W. W., et al., J. Acquired Immune Deficiency Syndromes and Human Retrovirology 14 (1997) 193-203.

Va. Further Therapeutic Uses

The isolated nucleic acid molecules and polypeptides of the invention may also be used therapeutically, for example in treating a cancer in an animal suffering therefrom. In such approaches, the goal of the therapy is to delay or inhibit the progression or growth of the cancer or tumor, to delay or inhibit the metastasis of the cancer or tumor, and/or to induce remission of the cancer or tumor. Particularly useful in this context are the polypeptides or fragments thereof containing a non-phosphorytable amino acid residue at a location corresponding to Y88 in the wildtype p27 sequence or the polypeptide encoded thereof or the nucleic acid molecules encoding these polypeptides. This is particularly a polypeptide comprising the amino acid sequence SEQ ID NO: 2, 4 or 6 or comprising the amino acid residues 1 to 95 of SEQ ID NO: 2 or comprising the amino acid residues 1 to 85 of SEQ ID NO: 4 or comprising the amino acid residues 1 to 100 of SEQ ID NO: 6 or comprising the amino acid residues 50 to 95 of SEQ ID NO: 2 or comprising the amino acid residues 38 to 85 of SEQ ID NO: 4 or comprising the amino acid residues 50 to 100 of SEQ ID NO: 6 characterized in that in the polypeptide derived from the polypeptide with the amino acid sequence SEQ ID NO: 2, the amino acid residue at position 88 and/or position 89 in SEQ ID NO: 2, or in the polypeptide derived from the polypeptide with the amino acid sequence SEQ ID NO: 4, the amino acid residue at position 77 in SEQ ID NO: 4 or in the polypeptide derived from the polypeptide with the amino acid sequence SEQ ID NO: 6, the amino acid residue at position 91 in SEQ ID NO: 6 is a non-phosphorylatable amino acid residue, preferably a phenylalanine residue. In another embodiment, this is a peptide fragment with a minimum length of 6 amino acids of a polypeptide comprising the amino acid sequence SEQ ID NO: 2, 4 or 6 characterized in that in the peptide fragment of a polypeptide comprising the amino acid sequence SEQ ID NO: 2, the peptide fragment comprises at least one of the flanking amino acid residues of the amino acid residue at position 88 or 89 in SEQ ID NO: 2 and the residue at position 88 and/or the residue at position 89 in SEQ ID NO: 2 is a non-phosphorylatable amino acid residue, preferably a phenylalanine residue, or in the peptide fragment of a polypeptide comprising the amino acid sequence SEQ ID NO: 4, the peptide fragment comprises at least one of the flanking amino acid residues of the amino acid residue at position 77 in SEQ ID NO: 4 and the residue at position 77 in SEQ ID NO: 4 is a non-phosphorylatable amino acid residue, preferably a phenylalanine residue, or in the peptide fragment of a polypeptide comprising the amino acid sequence SEQ ID NO: 6, the peptide fragment comprises at least one of the flanking amino acid residues of the amino acid residue at position 91 in SEQ ID NO: 6 and the residue at position 91 in SEQ ID NO: 6 is a non-phosphorylatable amino acid residue, preferably a phenylalanine residue.

Gene Therapy

In a first such aspect of the invention, the animal suffering from cancer may be treated by introducing into the animal one or more of the isolated nucleic acid molecules of the invention. This approach, known generically as “gene therapy,” is designed to express non-phosphorytable polypeptides according to the invention or fragments thereof in the cells making up the cancer or tumor and thereby to inhibit or delay the growth, progression or metastasis, or to induce the remission, of the tumor or cancer. Analogous gene therapy approaches have proven effective or to have promise in the treatment of other mammalian diseases such as cystic fibrosis (Drumm, M. L. et al., Cell 62:1227-1233 (1990); Gregory, R. J. et al., Nature 347:358-363 (1990); Rich, D. P. et al., Nature 347:358-363 (1990)), Gaucher disease (Sorge, J. et al, Proc. Natl. Acad. Sci. USA 84:906-909 (1987); Fink, J. K et al., Proc. Natl. Acad. Sci. USA 87:2334-2338 (1990)), certain forms of hemophilia (Bontempo, F. A. et al, Blood 69:1721-1724 (1987); Palmer, T. D. et al., Blood 73:438-445 (1989); Axelrod, J. H. et al., Proc. Natl. Acad. Sci. USA 87:5173-5177 (1990); Armentano, D. et al., Proc. Natl. Acad. Sci. USA 87:6141-6145 (1990)) and muscular dystrophy (Partridge, T. A. et al., Nature 337:176-179 (1989); Law, P. K et al, Lancet 336:114-115 (1990); Morgan, J. E. et al., J. Cell Biol. 111:2437-2449 (1990)), as well as in other treatments for certain cancers such as metastatic melanoma (Rosenberg, S. A. et al., Science 233:1318-1321 (1986); Rosenberg, S. A. et al., N. Eng. J. Med. 319:1676-1680 (1988); Rosenberg, S. A. et al., N. Eng. J. Med. 323:570-578 (1990)).

In a preferred such approach, one or more isolated nucleic acid molecules of the invention are introduced into or administered to the animal that is suffering from the cancer. Such isolated nucleic acid molecules may be incorporated into a vector or virion suitable for introducing the nucleic acid molecules into the cells or tissues of the animal to be treated, to form a transfection vector. Suitable vectors or virions for this purpose include those derived from retroviruses, adenoviruses and adeno-associated viruses. Alternatively, the nucleic acid molecules of the invention may be complexed into a molecular conjugate with a virus (e.g., an adenovirus or an adeno-associated virus) or with viral components (e.g., viral capsid proteins).

Techniques for the formation of vectors or virions comprising the nucleic acid molecules encoding the polypeptides according to the invention or fragments thereof are well-known in the art, and are generally described in “Working Toward Human Gene Therapy,” Chapter 28 in Recombinant DNA, 2nd Ed., Watson, J. D. et al., eds., New York: Scientific American Books, pp. 567-581 (1992). In addition, general methods for construction of gene therapy vectors and the introduction thereof into affected animals for therapeutic purposes may be obtained in the above-referenced publications, the disclosures of which are specifically incorporated herein by reference in their entirety. In one such general method, vectors comprising the isolated polynucleotides of the present invention are directly introduced into the cells or tissues of the affected animal, preferably by injection, inhalation, ingestion or introduction into a mucous membrane via solution; such an approach is generally referred to as “in vivo” gene therapy. Alternatively, cells, tissues or organs, particularly those containing cancer cells or tumors, may be removed from the affected animal, and placed into culture according to methods that are well-known to one of ordinary skill in the art; the vectors comprising the polynucleotides according to the invention may then be introduced into these cells or tissues by any of the methods described generally above for introducing isolated polynucleotides into a cell or tissue, and, after a sufficient amount of time to allow incorporation of the polynucleotides, the cells or tissues may then be re-inserted into an affected animal. Since the introduction of the nucleic acid molecules according to the invention are performed outside of the body of the affected animal, this approach is generally referred to as “ex vivo” gene therapy.

For both in vivo and ex vivo gene therapy, the isolated polynucleotides of the invention may alternatively be operatively linked to a regulatory DNA sequence, which may be a promoter or an enhancer, or a heterologous regulatory DNA sequence such as a promoter or enhancer derived from a different gene, cell or organism, to form a genetic construct as described above. This genetic construct may then be inserted into a vector, which is then directly introduced into the affected animal in an in vivo gene therapy approach, e.g., by intratumoral administration (i.e., introduction of the nucleic acid molecule or vector directly into a tumor in an animal, for example by injection), or into the cells or tissues of the affected animal in an ex vivo approach. In another preferred embodiment, the genetic construct of the invention may be introduced into the cells or tissues of the animal, either in vivo or ex vivo, in a molecular conjugate with a virus (e.g., an adenovirus or an adeno-associated virus) or viral components (e.g., viral capsid proteins; see WO 93/07283). Alternatively, transfected host cells, which may be homologous or heterologous, may be encapsulated within a semi-permeable barrier device and implanted into the affected animal, allowing passage of polypeptides of the invention into the tissues and circulation of the animal but preventing contact between the animal's immune system and the transfected cells (see WO 93/09222). These approaches result in increased production of polypeptides according to the invention by the treated animal via (a) random insertion of the nucleic acid molecules according to the invention into the host cell genome; or (b) incorporation of the nucleic acid molecules according to the invention into the nucleus of the cells where it may exist as an ertrachromosomal genetic element. General descriptions of such methods and approaches to gene therapy may be found, for example, in U.S. Pat. No. 5,578,461; WO 94/12650; and WO 93/09222.

Regardless of the approach used, however, use of these methods of the present invention will result in the increased production of nucleic acid molecules according to the invention by the cells and tissues of the treated animal, such that the cancer or tumor progression, growth or metastasis will be delayed or inhibited, or such that the cancer or tumor will go into remission or be cured.

Protein Therapy. In another preferred therapeutic approach provided by the present invention, an animal suffering from a cancer may be treated by administration of a composition comprising one or more of the isolated polypeptides of the invention to the affected animal, wherein such administration inhibits the progression, growth or metastasis, or induces the remission, of the cancer or tumor. In one such approach, the isolated polypeptides are administered to the animal in the form of the above-described pharmaceutical compositions. In another such approach, the compositions administered to the animal may comprise one or more of the isolated polypeptides having an amino acid sequence at least about 65%, or more preferably at least about 70%, 75%, 80%, 85%, 90%, 95% or 99%, identical to one or more of the above-described reference amino acid sequences. Thus, the invention further provides methods of treating an animal suffering from a cancer comprising administering to such an animal a pharmaceutical composition comprising a therapeutically effective amount of one or more isolated polypeptides of the invention and optionally a pharmaceutically acceptable carrier or excipient therefor. The invention further comprises a method for the preparation of a pharmaceutical composition for the treatment of cancer comprising a therapeutically effective amount of one or more isolated polypeptides of the invention and optionally a pharmaceutically acceptable carrier or excipient therefor. Further, the polypeptides of the invention can be used for the preparation of a pharmaceutical composition for the treatment of cancer. Further, the polypeptides of the invention can be used in medicine. The invention further contemplates a pharmaceutical composition comprising the polypeptides of the invention and optionally a pharmaceutically acceptable carrier or excipient therefor.

The polypeptide-containing compositions should be formulated and dosed in a fashion consistent with good medical practice, taking into account the clinical condition of the individual patient (especially the side effects of treatment), the site of delivery of the polypeptide composition, the method of administrations the scheduling of administration, and other factors known to practitioners. The “therapeutically effective amount” of the polypeptide for purposes herein is thus determined by such considerations. As a general proposition, the total therapeutically effective amount of polypeptide administered parenterally per dose will be in the range of about 0.01 μg/kg/day to about 1000 μg/kg/day of patient body weight, although, as noted above, this will be subject to therapeutic discretion. More preferably, this dose is at least 0.1 μg/kg/day, and most preferably for humans between about 0.1 and about 100 μg/kg/day for the polypeptide. If given continuously, the polypeptide may be administered either by 1-10 injections per day or by continuous subcutaneous infusion, for example, using a mini-pump. An intravenous bag solution may also be employed. The key factor in selecting an appropriate dose is the result obtained, as measured, for example, by increases in the circulating polypeptide level or by determining a decrease in tumor growth or metastasis or in remission of the cancer. Other useful measures of determining therapeutic effectiveness are known to one of ordinary skill in the art. The length of treatment needed to observe changes, and the interval following treatment for responses to occur, may vary depending on the desired effect.

Therefore, in an embodiment of the invention, a virus particle comprising a nucleic acid molecule or a vector according to the invention is provided and a mammalian cell comprising a nucleic acid molecule or a vector according to the invention. In another embodiment of the invention, a virus particle or a mammalian cell for use in medicine is provided, a pharmaceutical composition comprising a virus particle according to the invention or a mammalian cell according to the invention and a pharmaceutically acceptable carrier. In another embodiment of the invention, a virus particle according to the invention or a mammalian cell according to the invention is used for the preparation of a pharmaceutical composition for the treatment of cancer.

Pharmaceutical compositions for use in such methods comprise one or more of the isolated polypeptides of the present invention and may optionally comprise a pharmaceutically acceptable carrier or excipient therefor, as described above. The polypeptides, and the pharmaceutical compositions of the present invention, may be administered by any means described in the present application.

A variety of cancers may be treated in animals by these therapeutic methods of the invention. Cancers suitably treated by these methods include, but are not limited to, carcinomas, sarcomas, melanomas and leukemias, particularly those described above. The methods of the invention are particularly well-suited for treating cancers in any animal, preferably in mammals and most particularly in humans.

VI. Pharmacogenomics

Agents or modulators which have an effect on the expression of the phosphorylated polypeptide of the invention or which effect the phosphorylation of the non-phosphorylated polypeptide according to the invention can be administered to individuals to treat cancer in the patient. In conjunction with such treatment, the pharmacogenomics (i. e. the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus the pharmacogenomics of the individual permits the selection of effective agents (e.g. drugs) for prophylactic or therapeutic treatments based on a consideration of the individual's genotype. Such pharmacogenomics can further be used to determine appropriate dosages and therapeutic regimens. Accordingly the level of expression of the polypeptide of the invention in an individual can be determined to thereby select appropriate agent(s) for therapeutic or prophylactic treatment of the individual.

Therefore, the invention considers a method of selecting a composition for inhibiting the progression of cancer in a patient, the method comprising:
a) providing a sample comprising cancer cells from the patient;
b) separately exposing aliquots of the sample in the presence of a plurality of test compounds;
c) comparing expression of the polypeptide according to the invention or a peptide fragment according to the invention in each of the aliquots; and
d) selecting one of the test compositions which alters the level of expression of the polypeptide in the aliquot containing that test composition, relative to other test compositions.

Particularly preferred in this context is also to determine the level of phosphorylated vs. unphosphorylated polypeptide according to the invention with and without test compound.

Pharmacogenomics deals with clinically significant variations in the response to drugs due to altered drug disposition and abnormal action in affected persons. See e.g. Linder, M. W., et al, Clin. Chem. 43 (1997) 254-266. In general two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of Lava beans.

As an illustrative embodiment the activity of drug metabolizing enzymes is a major determinant of both the intensity and duration of drug action. The discovery of genetic polymorphisms of drug metabolizing enzymes (e.g. N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug.

These polymorphisms are expressed in two phenotypes in the population the extensive metabolized (EM) and poor metabolizes (PM). The prevalence of PM is different among different populations. For example the gene coding for CYP2D6 is highly polymorphic and several mutations have been identified in PM which all lead to the absence of functional CYP2D6. Poor metabolizers of CYP2D6 and CYP2C19 quite frequently experience exaggerated drug response and side effects when they receive standard doses. If a metabolite is the active therapeutic moiety a PM will show no therapeutic response as demonstrated for the analgesic effect of codeine mediated by its CYP2D6 formed metabolite morphine. The other extreme are the so called ultra-rapid metabolizers who do not respond to standard doses. Recently the molecular basis of ultra-rapid metabolism has been identified to be due to CYP2D6 gene amplification.

Thus the level of expression of the polypeptide according to the invention in an individual can be determined to thereby select appropriate agent(s) for therapeutic treatment of the individual. In addition pharmacogenetic studies can be used to apply genotyping of polymorphic alleles encoding drug-metabolizing enzymes to the identification of an individual's drug responsiveness phenotype. This knowledge when applied to dosing or drug selection can avoid adverse reactions or therapeutic failure and thus enhance therapeutic or prophylactic efficiency when treating a subject with a modulator of expression of the polypeptide of the invention.

VII. Monitoring Clinical Trials

Monitoring the influence of agents (e.g. drug compounds) on the level of expression of the polypeptide of the invention can be applied not only in basic drug screening, but also in clinical trials. In a preferred embodiment the present invention provides a method for monitoring the effectiveness of treatment of a subject with an agent (e.g. an agonist, antagonist, peptidomimetic, protein, peptide, nucleic acid, small molecule or other drug candidate) comprising the steps of (i) obtaining a pre-administration sample from a subject prior to administration of the agent, (ii) detecting the level of expression of the phosphorylated polypeptide according to the invention in the pre-administration sample, (iii) obtaining one or more post-administration samples from the subject, (iv) detecting the level of expression of the nucleic acid molecule in the post-administration samples, (v) comparing the level of expression of the polypeptide in the pre-administration sample with the level of expression of the polypeptide in the post-administration sample or samples and (vi) altering the administration of the agent to the subject accordingly. For example increased administration of the agent can be desirable to increase expression of the polypeptide to higher levels than detected i.e. to increase the effectiveness of the agent. Alternatively decreased administration of the agent can be desirable to decrease expression of the polypeptide according to the invention to lower levels than detected i. e. to decrease the effectiveness of the agent. Further embodiments useful for monitoring clinical trials were described under IV. Predictive medicine of the present application.

The following examples, references, sequence listing and figures are provided to aid the understanding of the present invention the true scope of which is set forth in the appended claims. It is understood that modifications can be made in the procedures set forth without departing from the spirit of the invention.

### DESCRIPTION OF THE FIGURES

FIG. 1: Sequence alignment between the sequences of human p27, p21 and p57. Numbers above refer to the amino acids of p27. (Program Gene Jockey II, Fixed and floating gap penalty: 10; PAM 250; Gap penalty: 5; >Window size: 5; Number of best diagonals: 3 ktupe:1.

FIG. 2: p27^{Kip1} is phosphorylated on tyrosine-88 in vitro and in vivo.

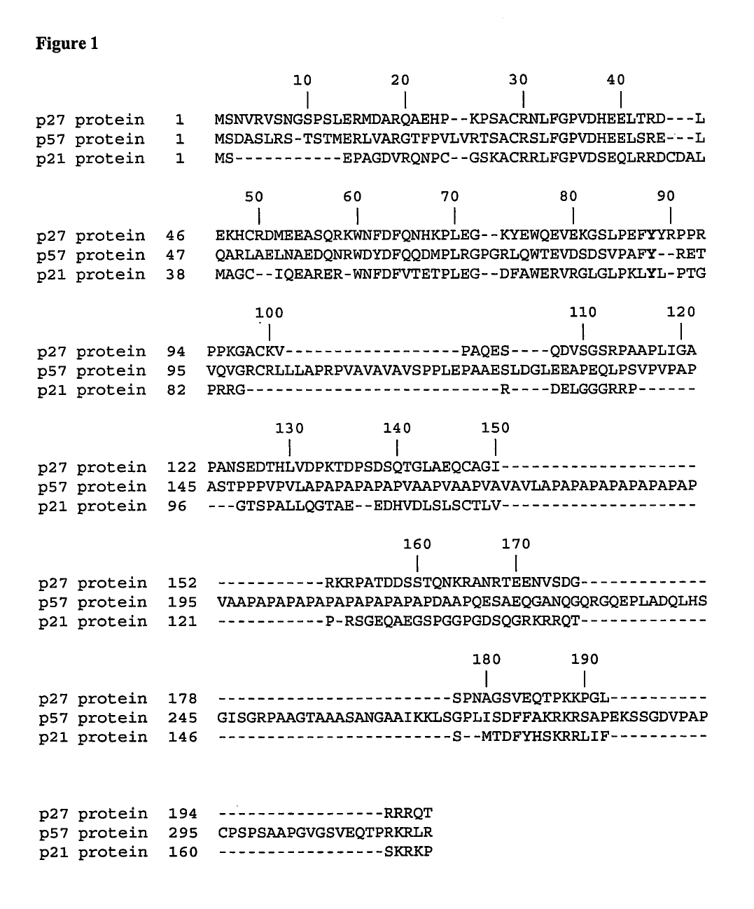

a. 2D phosphoamino acid analysis of immunoprecipitated p27. HeLa cells transfected with p27 were labeled for 4 h with ³²P ortho-phosphate. p27 was immunoprecipitated, purified, hydrolyzed and subjected to 2-dimensional electrophoresis on a thin layer cellulose plate. The autoradiograph shows phosphate incorporation into serine, threonine and tyrosine; partially hydrolysed phosphopeptides are indicated by asterisks. The migration of standards representing phospho-serine, -threonine or -tyrosine is shown after ninhydrin staining (right).

b. Lyn and Abl phosphorylate p27 tyrosine-88 in vitro. Equal amounts of p27 (schematically represented in the upper panel: the CDK-inhibitory domain is indicated in dark grey) or mutant p27(indicated tyrosine residues exchanged to phenylalanine) were incubated with recombinant constitutive active Lyn kinase or the SH3 and kinase domain of the c-abl kinase at 30° C. in the presence of γ-³²P-ATP. Proteins were separated by SDS-PAGE and detected by coomassie staining (upper panel). The amount of incorporated ³²P was shown in the autoradiography (lower panel).

c. p27 is phosphorylated on tyrosine-88 in vivo. HA-tagged p27 was coexpressed in 293T cells with Lyn or Bcr-Abl kinase. Anti-HA-immunoprecipitates were incubated with or without calf intestinal alkaline phosphatase (CIAP), and a Western blot with anti-phosphoY88 p27 antibodies was performed. The same blot was reprobed with a HRP-conjugated anti-p27 antibody. d. p27-tyrosine phosphorylation is enhanced by overexpression of Lyn or Bcr-Abl kinase. MCF7 cells transiently coexpressing either the HA-tagged p27 or mutant HA-tagged p27 (Y88F, Y89F) and Bcr-Abl or Lyn kinase were treated with orthovanadate for 4 hours. Tagged p27 species were immunoprecipitated with an anti-HA antibody, and probed with anti-phospho Y88 p27 antibodies. The same membrane was reprobed with an HRP-conjugated anti-p27 antibody.

FIG. 3: p27 is phosphorylated on Tyr-88 in p210 Bcr-Abl transformed human cells. a STI-571 prevents Bcr-Abl but not by Lyn-dependent p27 tyrosine phosphorylation in transfected 293T cells. HA-tagged p27 was transfected in 293T cells with a Bcr-Abl or Lyn encoding plasmids. Cells were treated with 10 μM STI-571 for the indicated times. Equal amounts of cell lysates were analyzed by Western blotting with monoclonal anti-phospho-Y88 antibodies and HRP-conjugated anti-p27 antibodies as indicated. b. STI-571 prevents p27 tyrosine phosphorylation in K562 cells. p27 was immunoprecipitated from K562 cells treated with 10 μM STI-571 for the indicated times. Western blot analysis was performed with monoclonal anti-phospho-Y88 and HRP-conjugated anti-p27 antibodies. c. STI-571 increases p27 level in K562 cells. K562 cells were treated with 2 or 10 μM STI-571 for 16 hours, and equal amounts of proteins were analyzed in Western blotting utilizing anti-p27 and anti-PSTAIRE antibodies. d. STI-571 increases the stability of p27 in K562 cells. K562 cells were mock-treated or treated with 10 μM STI-571 for 16 hours. Cells (1×10⁷/ml) were labeled with a ³⁵S labeled methionine/cysteine mix for 1 hour, and subsequently incubated in the absence of radioactivity for the indicated times. p27 was immunoprecipitated using anti-p27 antibodies. The labeled protein was visualized by autoradiography. The intensity of the bands was determined using a PhosphorImager and is expressed as a fraction of the time point 0. The experiment was repeated three times with similar results. One representative experiment is shown.

FIG. 4: Tyrosine-88 phosphorylated p27 binds CDK complexes, but is a poor CDK inhibitor.

a. Gel filtration analysis. Tyrosine-phosphorylated and unphosphorylated recombinant p27 were mixed in a ratio of 1:1 and subsequently incubated with purified CDK2/CyclinA complexes. Protein complexes were separated by gel filtration from unbound p27. The fractions were analysed by Coomassie staining for protein abundance (upper panel) and by Western blotting (lower panels). The membrane was probed with anti-phospho-tyrosine antibodies (pY) reacting with tyrosine-phosphorylated p27 and the SH3-kinase domain of c-Abl, an antibody against pY88 of p27, detecting only Y-88 phosphorylated p27 and a HPR-coupled anti-p27 antibody detecting total p27 level, as indicated.

b. Tyr-88 phosphorylated p27 binds cyclin D and cyclin E complexes in vivo. Cyclin D1 and cyclin E1 complexes were immmunoprecipitated from K562 cell lysates. Precipitates were analyzed using anti-phospho-Y88 p27 and HRP-conjugated anti-p27 antibodies.

c. CDK2 inhibition of tyrosine-phosphorylated and unphosphorylated p27. p27 or mutant p27 lacking all tyrosine residues (Y-F) were treated with recombinant constitutive active Abl kinase. Equal amounts of phosphorylated p27, mutant p27 or mock treated p27 were compared in their ability to inhibit cycinA/CDK2 kinase in histone H1 kinase assays. The incorporation of ³²P into histone H1 is detected after SDS-PAGE by autoradiography.

FIG. 5: Tyr-88 phosphorylated p27 is efficiently phosphorylated by CDK2 kinase on Thr-187. a. Kinase assay of p27 bound to cyclin A/CDK2. Recombinant wild-type p27 and mutant p27 lacking all tyrosines (p27 Y-F) was incubated with constitutive active recombinant c-Abl SH3-kinase domain where indicated (+). p27 was purified and equal amounts (Coomassie staining, upper panel) were incubated in excess with active CDK2/cyclinA for 30 min at 30° C. in the presence of γ-³²P-ATP. The amount of ³²P incorporated into p27 was detected by autoradiography (lower panel).

b. Tyrosine-88 phosphorylated p27 is a better substrate for CDK2 kinase. Equal amounts (1 μg) of recombinant wild-type p27 and mutant p27 (Y88F or Y89P) were tyrosine-phosphorylated by recombinant Abl-kinase if indicated (+) and incubated in excess with purified CDK2/cyclinA complex at 37° C. for 5 min. The amount of p27 phosphorylation at threonine-187 by CDK2 was tested in Western blotting using a phospho-threonine-187 specific antibody. The same membrane was reprobed with an HRP-conjugated anti-p27 antibody. The tyrosine-phosphorylation status of the p27 used in this assay was analyzed in separate western blots utilizing anti-phospho Y88- or anti-phospho Y89-specific antibodies.

FIG. 6: Metabolic labelling of adherent HeLa cells with [³²P] -ortho-phosphate and thin layer chromatographic analysis of the phosphoamino acids of overexpressed and stabilized p21 and p27.

(A) A 250 ml bottle of adherent HeLa cells was transfected with p21 and p27 and 24 h after transfection it was metabolically labelled for 4 h with 5 mCi (0.5 mCi/ml) [³²P] -ortho-phosphate. The cells were additionally cultured in the presence of MG132, LLnL, ocadeic acid and Na ortho-vanadate to inhibit the proteasome and cell phosphatases. During the last 30 min of the metabolic labelling the cells were also cultured with Na meta-vanadate. p21 and p27 were immunoprecipitated by specific antibodies. The mixtures were separated on SDS-PAGE and blotted on a PVDF membrane. The membrane was exposed to an X-ray film (lane 1a and 2a) and the membrane was superimposed on radioactive signals of the film. p21 and p27 were cut out of the membrane. The remaining membrane was again exposed to verify the excised proteins (lane 1b and 2b).

(B) Two-dimensional separation of the phosphoamino acids of overexpressed p21 and p27: The membrane fragments were rehydrated in methanol, washed and treated for 1 h with 6 N HCl at 110° C. The resulting protein hydrolysate was removed and dried under a vacuum, dissolved in the mobile buffer of the first dimension and applied to a cellulose thin layer plate together with a phosphoamino acid standard. The phosphoamino acids were separated electrophorectically in two dimensions. The first dimension was carried out for 35 min at pH 1.9 and 1.5 kV. The plate was dried and the amino acids were separated for 30 min in the second dimension at pH 3.5 and 1.3 kV. The phosphoamino acid standard was subsequently made visible by ninhydrin staining. The phosphorylated amino acids were detected by exposing the cellulose plate to an X-ray film.

FIG. 7: Comparison of the ability to phosphorylate the tyrosines of the Cip/Kip inhibitor proteins in vitro.

p21, p27 and p57 that were produced in E. coli and purified, were phosphorylated with the recombinant, shortened tyrosine kinase Abl^{KD+SH3}. The kinase reaction was carried out for 2 h at 30° C.

The effectiveness of the phosphorylation of the various inhibitor proteins was estimated by incorporating [γ³²P]ATP into the respective protein. The nucleotide exchange factor RanGAP1 and the BSA which was added to the kinase reactions for stabilization served as controls of the specificity of phosphorylation. The Coomassie-stained gel of the kinase reactions (top) and the radiogram of the dried gel (bottom) are shown.

FIG. 8: Enhanced stability of a p27 Y88F mutant MEF cells heterozygous deleted for p27 were infected with retroviruses expressiong either p27 or a mutant p27-Y88F. Cells were pulse labelled and subsequently chased as described. p27 was immunoprecipitated and the amount of remaining labelled p27 was determined using a phosphorImager. The half life for both proteins determined in this experiment is indicated below.

FIG. 9: p27 Y88F arrests K562 CML cells in G1 phase of the cell cycle. K562 CML cells were transfected with GFP and either 25 ug of p27 or 25 ug of p27-Y88F expression vector. 48 hours after transfection, cells with similar GFP expression level were isolated by flow cytometry cell sorting and the cell cycle distribution of these cells was determined using BrdU incorporation and PI staining. The distribution of these cells in the cell cycle phases is shown. Only p27-Y88F expression leads to an accumulation of G1 phase cells and reduced cells in S-phase. In contrast, expression of p27 did not significantly alter the cell cycle profile of these BCR-Abl-trannsformed human tumor cells.

### EXAMPLE

The CDK inhibitor p27^{Kip1} controls cell proliferation by binding to and regulating the activity of cyclin-dependent kinases (Sherr, C., and Roberts, J. M., Genes Dev. 13 (1999) 1501-1512; Hengst, L., and Reed, S. I., Curr. Top. Microbiol. Immunol. 227 (1998) 25-41). The following example shows that the conserved tyrosine residue 88 in the CDK binding domain of p27 is phosphorylated by the Lyn and Bcr-Abl kinases. This phosphorylation does not prevent p27 binding to the CDK/cyclin complex but impairs its CDK inhibition. Importantly, the tyrosine phosphorylated inhibitor becomes efficiently phosphorylated in CDK2 kinase complexes on threonine-187, a site required for the SCF-Skp2 dependent degradation of p27 at the G1/S transition³. A mutant of p27 where tyrosine 88 is exchanged to phenylalanine (Y88F) is stabilized. Whereas moderate overexpression of p27 failed to arrest the cell cycle of K562 CML cells, overexpression of the Y88F mutant leads to increased cell cycle arrest in G1 phase and a dramatic decline in S-phase. This direct link between oncogenic tyrosine kinases and p27 provides an explanation for premature p27 elimination in cells transformed by activated tyrosine kinases. Tyrosine 88 of p27 is highly conserved in eucaryotic Cip/Kip proteins. The related inhibitors in humans, p21 and p57, can also become phosphorylated on tyrosines, suggesting a conserved mechanism.

### Example—Materials and Methods

General

If not stated otherwise the described methods were derived from Sambrook et al. (1989), Current Protocols in Molecular Biology, Current Protocols in Protein Science and Current Protocols in Cell Biology. Further description of the methods and materials can be found in the European patent application EP03024165.7 filed on Oct. 20, 2003 accessible through file inspection at the European Patent Office and the Ph.D. thesis by Dr. Matthias Grimmler available from the Ludwig-Maximilians-Universität München, München, Germany.

Materials

Chemicals and consumables

If not stated otherwise chemicals and fine chemicals were obtained in analytical quality from Merck (Darmstadt), Sigma-Aldrich (Taufkirchen), SERVA (Heidelberg) and Roth (Karlsruhe). The water used in this study was prepared on a “Purelab Plus UV/UF” filter system from the USF Company (Ransbach—Baumbach). It is referred to in the following as ddH₂O.

Radiochemicals

Radiochemicals were obtained from Amersham Pharmacia Biotech (Freiburg).

Materials for filtration and dialysis

| | |
|---|---|
| Centricon 5, 10 and 30 | Amicon, Witten |
| Diaflow ultrafiltration membranes | Amicon, Witten |
| MF-Millipore membrane filter | Sigma-Aldrich, Taufkirchen |
| Spectra-Por, dialysis tubes | The Spectrum Companies, |
| | Gardena, USA |
| UH 100/10 and 30 ultra cartridges | Schleicher & Schüll, Dassel |
| Whatman 3 MM, filter paper | Maidstone, UK |
| Whatman GF/C, glass fibre filter | Maidstone, UK |
| centrifuge filter units 5K, 10K and 30K | Sigma-Aldrich, Taufkirchen |

Chromatography matrices and ready-packed chromatography columns

| | |
|---|---|
| DC plates 20 × 20 cm, Cellulose | Merck, Darmstadt |
| Glutathione Sepharose 4B | Amersham Pharmacia Biotech, Freiburg |
| High Trap Q 1 ml and 5 ml | Amersham Pharmacia Biotech, Freiburg |
| High Trap SP 1 ml and 5 ml | Amersham Pharmacia Biotech, Freiburg |
| MonoQ HR 5/5 | Amersham Pharmacia Biotech, Freiburg |
| ProBond Nickel-Chelating Resin | Invitrogen, Groningen, Netherlands |
| protein A Sepharose CL-4B | Sigma-Aldrich, Taufkirchen |
| protein G Sepharose 4 fast flow | Amersham Pharmacia Biotech, Freiburg |
| Sepharose-SP fast flow | Sigma-Aldrich, Taufkirchen |
| Sepharose-Q fast flow | Sigma-Aldrich, Taufkirchen |
| Sephasil C4μm ST 4.6/250 | Amersham Pharmacia Biotech, Freiburg |
| protein C4 | |
| Superdex 200 HR 10/30 | Amersham Pharmacia Biotech, Freiburg |
| Superdex 200 pg HiLoad 16/30 | Amersham Pharmacia Biotech, Freiburg |
| Superdex 200 pg HiLoad 26/60 | Amersham Pharmacia Biotech, Freiburg |

Molecular weight and length standards

| | |
|---|---|
| BenckMark Protein Ladder, protein size standard | Life Technologies, |
| | Karlsruhe |
| GeneRuler 100 bp Ladder Plus, DNA size standard | MBI Fermentas, |
| | St. Leon-Rot |
| GeneRuler 1 kb Ladder Plus, DNA size standard | MBI Fermentas, |
| | St. Leon-Rot |
| IEF Markers 3-10, SERVA liquid mix | Invitrogen, |
| | Groningen, |
| | Netherlands |
| MW-FG-200, gel filtration marker 12-200 kDa, | Sigma-Aldrich, |
| | Taufkirchen |

Complex reagents and reaction sets (kits)

| | |
|---|---|
| Big Dye Terminator Cycle Sequencing RR-Mix | Applied Biosystems, Foster City, |
| | USA |
| Complete without EDTA, protease inhibitor mix, | Roche Mannheim |
| Deoxynucleotide set | Sigma-Aldrich, Taufkirchen |
| Effectene, Transfection reagent | QIAGEN, Hilden |
| Freund's Adjuvant, incomplete | Sigma-Aldrich, Taufkirchen |
| IPG buffer, pH 3-10 and pH 6-11 | Amersham Pharmacia Biotech, Freiburg |
| Micro BCA Protein Assay Reagent Kit | Pierce, Rockford, USA |
| pGEM-T Easy Vector System I and II | Promega, Mannheim |
| QIAGEN Plasmid Kit (Mini, Midi) | QIAGEN, Hilden |
| Rotiphorese Gel 30 | Roth, Karlsruhe |
| Super Signal West Femto Maximum Sensitivity Substrate | Pierce, Rockford, USA |
| TA Cloning Kit | Invitrogen, Groningen, Netherlands |
| TiterMax Gold, Adjuvant | CytRxCorporation, Norcross, USA |
| TNT-SP6/T7 Coupled Reticulocyte Lysate System | Promega, Mannheim |
| TOPO TA Cloning Kit | Invitrogen, Groningen, Netherlands |

Buffers and solutions

| | |
|---|---|
| Amido black staining | 0.2% (w/v) amido black 10B, 10% (v/v) |
| solution | methanol, 2% (v/v) acetic acid |
| block solution | 2% (w/v) BSA in PBS |
| complete without | 1 tablet complete without EDTA - protease |
| EDTA, 50 x | inhibitor mix (Roche) in 1 ml ddH₂O |
| Coomassie staining | 50% (v/v) methanol, 10% (v/v) acetic acid, |
| solution | 0.1% (w/v) SERVA Blue R (SERVA) |
| TLC buffer I | 50 ml (88%) formic acid, 156 ml glacial acetic |
| | acid make up to 2000 ml with ddH₂O, do not |
| | adjust pH which should be about 1.8-1.9 |
| TLC buffer II | 100 ml glacial acetic acid, 10 ml pyridine, 0.5 |
| | ml EDTA make up to 2000 ml with ddH₂O, |
| | adjust the pH to 3.5 with pyridine |
| IP buffer | 200 mM NaCl, 50 mM Tris, pH 7.5, 0.25% |
| | (w/v) NP-40, 5 mM EDTA and 1 mM PMSF, |
| | 10 μg/ml Aprotinin, leupeptin, pepstatin A |
| Kinasation buffer I, | 200 mM Tris/HCl, 75 mM MgCl₂, pH 7.2 |
| 10 x | |
| Kinasation buffer II, | 20 mM HEPES pH 7.5, 5 mM MgCl2, 1 mM |
| | MnCl₂, 0.05% (w/v) NP-40, 7.15 mM beta- |
| | mercaptoethanol, 125 μM Na ortho-vanadate, |
| | 10 μM PMSF, 0.1 μg/ml A, P, LP |
| NET gelatin block | 1.5 M NaCl, 0.05 M EDTA, 0.5 M Tris pH 7.5, |
| buffer, 10 x | 0.5% (w/v) Triton-X-100, 26 g/l gelatin, |
| | adjust to pH 7.5 |
| P1 | 50 mM Tris/HCl, 10 mM EDTA, 100 μg/ml |
| | RNase A, pH 8.0 |
| P2 | 200 mM NaOH, 1% (w/v) SDS |
| P3 | 3 M K acetate, pH 5.5 |
| PBS | 140 mM NaCl, 2.7 mM KCl, 10 mM |
| | Na₂HPO₄, 1.8 mM KH₂PO4, adjust pH to 7.5 |
| | with NaOH |
| PBS-T | 0.05% (w/v) Tween-2O in PBS |
| PI staining solution | 1 μg/ml propidium iodide, 0.1% (v/v) |
| | Triton-X-100, 0.2 mg/ml RNase A in PBS |
| RIPA buffer | 50 mM Tris/HCl, 150 mM NaCl, 1 mM |
| | EDTA, 1% (v/v) Nonidet P 40, 0.25% (w/v) |
| | sodium deoxycholate, 0.1% (w/v) SDS, pH |
| | 7.5 |
| SDS electrophoresis | 25 mM Tris, 192 mM glycine, 0.1% (w/v) |
| buffer | SDS |
| SDS sample buffer, | 209 mM Tris/HCl, 41% (w/v) glycerol, 7.7% |
| 10 x | (w/v) SDS, 0.003% (w/v) bromo-phenol |
| | blue, 17% (v/v) beta-mercaptoethanol, pH |
| | 6.8 |
| SDS sample buffer, | 125 mM Tris/HCl, 17% (w/v) glycerol, 4.1% |
| 2 x | (w/v) SDS, 0.001% (w/v) bromo-phenol |
| | blue, 2% (v/v) beta-mercaptoethanol, pH 6.8 |
| SDS collecting gel | 0.5 M Tris/HCl, 0.4% (w/v) SDS, pH 6.8 |
| buffer, 4 x | |
| SDS separating gel | 1.5 M Tris/HCl, 0.4% (w/v) SDS, pH 8.8 |
| buffer, 4 x | |
| TBE | 90 mM Tris, 90 mM boric acid, 2 mM EDTA |
| TBS | 20 mM Tris/HCl, 150 mM NaCl, pH 7.5 |
| TE | 10 mM Tris/HCl, 1 mM EDTA, pH 8.0 |
| Triton-X-100 solution | 0.2% (w/v) Triton-X-100 in PBS |
| transfer buffer | 47.9 mM Tris, 38.6 mM glycine, 0.037% |
| | (w/v) SDS, 20% (v/v) methanol |
| TSS | 10% (w/v) polyethylene glycol 6000, 5% |
| | (v/v) DMSO, 50 mM MgCl₂ in LB medium |

Media components and media additives

| | |
|---|---|
| 5-bromo-2-deoxyuridine | Sigma-Aldrich, Taufkirchen |
| stock solution 1% (w/v) in PBS | |
| L-cysteine | Sigma-Aldrich, Taufkirchen |
| stock solution 25 mg/ml in PBS | |
| Dulbeccos's Modified Eagle's Medium (DMEM) | Life Technologies, Karlsruhe |
| containing 4500 mg/l glucose, without sodium pyruvate | |
| DMEM containing 4500 mg/ml glucose, without L-glutamine, | Life Technologies, |
| | Karlsruhe |
| sodium pyruvate, L-cysteine, L-methionine | Sigma-Aldrich, Taufkirchen |
| foetal calf serum (FCS) and | |
| newborn calf serum (NCS) | |
| L-glutamine (100 x) | Life Technologies, Karlsruhe |
| MEM-Minimum Essential Medium | Life Technologies, Karlsruhe |
| MEM-Non-essential amino acid solution | Life Technologies, Karlsruhe |
| L-methionine | Sigma-Aldrich, Taufkirchen |
| Minimum Essential Medium Eagle, w/o Na-phosphate | Sigma-Aldrich, Taufkirchen |
| stock solution 25 mg/ml in PBS | |
| sodium pyruvate (100 x) | Life Technologies, Karlsruhe |
| Nocadazole | Sigma-Aldrich, Taufkirchen |
| stock solution 5 mg/ml in DMSO | |
| penicillin/streptomycin (100 x) | Life Technologies, Karlsruhe |
| Sf-900 II SFM Basal Powdered Medium | Life Technologies, Karlsruhe |
| Sf-900 II Supplement | Life Technologies, Karlsruhe |
| thymidine | Sigma-Aldrich, Taufkirchen |
| stock solution 200 mM in PBS | |
| trypsin/EDTA (10 x) | Life Technologies, Karlsruhe |

Media composition

| | |
|---|---|
| standard medium | DMEM containing 4500 mg/l glucose, 10% (v/v) FCS, |
| for HeLa, 293T | 100 U/ml penicillin, 100 μg/ml streptomycin |
| and MCF7 | DMEM containing 4500 mg/l glucose, 10% (v/v) FCS, |
| for HS68 | 100 U/ml penicillin, 100 μg/ml streptomycin, |
| | 1 mM sodium pyruvate |
| Chase medium | standard medium containing 0.1 mg/ml L-methionine, |
| | 0.12 mg/ml L-cysteine, 25 mM HEPES/KON, pH 7.4 |
| Hunger medium | DMEM containing 4500 mg/l glucose, 10% (v/v) FCS, |
| | 100 U/ml penicillin, 100 μg/ml streptomycin, 25 mM |
| | HEPES/KOH, pH 7.4, without sodium pyruvate, |
| | L-cysteine, L-methionine |
| Phospholabel | Minimum essential Eagle's medium containing Earls' |
| medium | salts, L-glutamine, NaHCO₃ and containing 4.5 g/l |
| | glucose, 2% (v/v) FCS without sodium phosphate |
| Puls-Medium | Hunger medium containing 0.05 mCi “Pro-mix”/ml |
| | 4.5 g/l glucose and MEM non-essential amino acid |
| | solution, 10% (v/v) FCS |

Antibodies and primary antibodies:

| | |
|---|---|
| anti-AbI 1 (clone 24-11) | Santa Cruz Biotechnology, Heidelberg |
| anti-BrdU (clone B44), coupled to FITC | Becton Dickinson, Hamburg |
| anti-Cyclin A (T310), rabbit antiserum | Hengst et al., 1994 |
| | directed against human cyclin anti- |
| | cyclin D1 (clone DCS-6) |
| anti-cyclin E1 (clone HE12) | Progen, Heidelberg, |
| | Neomarkers Fremont, USA |
| anti-GST, rabbit antiserum directed against GST | Laboratory of Ludger Hengst |
| anti-HA.11 (clone 16B12) | BabCo, Richmond, USA |
| anti-Lyn (clone 42) | Becton Dickinson, Hamburg |
| anti-p21 (clone CP36 and CP74, mixed) | Upstate Biotechnology, Eching |
| anti-p21, rabbit antiserum directed against | Santa Cruz Biotechnology, |
| the last 19 C-terminal aa from human p21 | Heidelberg |
| anti-p21, goat antiserum directed against p21 | this study |
| anti-p27/Kip1 (clone 57) | Becton Dickinson, Hamburg |
| anti-p27, rabbit antiserum directed against | Santa Cruz Biotechnology, |
| the last 19 C-terminal aa of human p27 | Heidelberg |
| anti-p27 (G96) goat antiserum | this study |
| directed against p27 anti-p27-pY88 | this study |
| anti-p57, rabbit antiserum directed against | Santa Cruz Biotechnology, |
| the last 20 C-terminal aa of human p57, | Heidelberg |
| anti-PSTAIRE, monoclonal antibody directed | Dulic et al., 1992 |
| against the PSTAIRE region of cyclin-dependent kinases | |
| anti-phospho-p27, rabbit antiserum directed | Upstate Biotechnologies, Eching |
| against human, on T187 phosphorylated p27 | |
| anti-phospho-p27, rabbit antiserum directed | Zymed Laboratories, South |
| against human, on T187 phosphorylated p27 | San Francisco, USA |
| anti-phospho-tyrosine (clone 4G10) | Upstate Biotechnologies, Eching |
| anti-phospho-tyrosine (clone P-Tyr-100) | Cell Signaling Technology, |
| | Frankfurt am Main |
| anti-Src, rabbit antiserum directed | Santa Cruz Biotechnology |
| against human Src | Heidelberg |
| anti-γ-tubulin (clone DM 1A) | Sigma-Aldrich, Taufkirchen |

Secondary antibodies:

| | |
|---|---|
| goat anti-mouse IgG, (H + L), | Pierce, Rockford, USA |
| peroxidase-coupled, | |
| goat anti-rabbit IgG, (H + L), | Pierce, Rockford, USA |
| peroxidase-coupled, | |
| anti-goat IgG, peroxidase-coupled | Santa Cruz Biotechnology, |
| | Heidelberg |
| goat anti-mouse IgG, (H + L), FITC-coupled | Jackson ImmunoResearch |
| | Laboratories, West Grove, |
| | USA |
| goat anti-rabbit IgG, (H + L), FITC-coupled | Jackson ImmunoResearch |
| | Laboratories, West Grove, |
| | USA |
| goat anti-mouse IgG, (H + L), | Jackson ImmunoResearch |
| rhodamine-coupled | Laboratories, West Grove, |
| | USA |
| goat anti-rabbit IgG, (H + L), | Jackson ImmunoResearch |
| rhodamine-coupled | Laboratories, West Grove, |
| | USA |

Enzymes:

Enzymes were obtained from Calbiochem-Novabiochem GmbH (Bad Soden), New England Biolabs (Schwalbach/Taunus), MBI Fermentas (St. Leon-Rot), Promega (Mannheim), QIAGEN (Hilden), Roche (Mannheim) and Stratagene (Heidelberg). If not stated otherwise, the reaction conditions given by the manufacturer were used.

DNA Oligonucleotides:

DNA oligonucleotides were obtained from MWG-Biotech (Ebersberg) and Metabion (Martinsried).

Primer DNA oligonucleotides for the amplification and mutagenesis of p27 and p27 fragments:

| | |
|---|---|
| Primer | DNA sequence |

| | |
|---|---|
| LHSD37 | 5′-GGATCCGGGAGACATATGTCAAACGTGCG-3′ |
| (SEQ ID | |
| NO: 7) | |
| LHSD40 | 5′-GGAGTCTTCTGCAGTTTGCATTACTATCCCTAGG-3′ |
| (SEQ ID | |
| NO: 8) | |
| p27-F/Y2, | 5′-GCTTGCCCGAGTTCTATTTCAGACCCCCGCGG-3′ |
| Re-FD | |
| (SEQ ID | |
| NO: 9) | |
| p27-F/Y2, | 5′-CCGCGGGGGTCTGAAATAGAACTCGGGCAAGC-3′ |
| Re-Rev | |
| (SEQ ID | |
| NO: 10) | |
| p27-F/Y3, | 5′-GCTTGCCCGAGTTCTTCTACAGACCCCCGCGGCC-3′ |
| Re-FD | |
| (SEQ ID | |
| NO: 11) | |
| p27-F/Y3, | 5′-GGCCGCGGGGGTCTGTAGAAGAACTCGGGCAAGC-3′ |
| Re-Rev | |
| (SEQ ID | |
| NO: 12) | |
| p27-Y1 FD | 5′-CCCCTAGAGGGCAAGTTCGAGTGGCAAGAG-3′ |
| (SEQ ID | |
| NO: 13) | |
| p27-Y1 | 5′-CTCTTGCCACTCGAACTTGCCCTCTAGGGG-3′ |
| Rev | |
| (SEQ ID | |
| NO: 14) | |
| p27-Y2Y3B | 5′-GCCGCGGGGGTCTGAAGAAGAACTCGG-3′ |
| (SEQ ID | |
| NO: 15) | |

Primer DNA oligonucleotides for the amplification of c-Abl and of c-Abl fragments:

| | |
|---|---|
| Primer | DNA sequence |

| | |
|---|---|
| Abl-inc SH3 | 5′-CATATGCCCAACCTTTTTGTGGCA-3′ |
| (SEQ ID NO: 16) | |
| Abl Kin.Dom.-ATG | 5′-CATATGGTCAACAGCCTGGAGAAAC-3′ |
| (SEQ ID NO: 17) | |
| Abl Kin.Dom.-TGA | 5′-CTTCAGCAGGTTCTGGTCTTGGTG-3′ |
| (SEQ ID NO: 18) | |
| Abl-NdeI-Exon IB | 5′-GCATATGGGGCAGCAGCCTGG-3′ |
| (SEQ ID NO: 19) | |
| Abl-TAG | 5′-GCAGGCGGATCCGACGGGCC-3′ |
| (SEQ ID NO: 20) | |

Plasmids

| | |
|---|---|
| pApuro | Laboratory of Markus Warmuth, Hämatologikum |
| | der GSF, Groβhadern |
| pBluescript SK (—) | Stratagene, Heidelberg |
| pCR2 | Invitrogen, Groningen, Netherlands |
| pCRII | Invitrogen, Groningen, Netherlands |
| pCruz-HA | Santa Cruz, Heidelberg |
| pET-3a,-11a,-24d,-28a | Calbiochem-Novabiochem GmbH, Bad Soden |

Cell lines

| | |
|---|---|
| HeLa, suspension | human cervical carcinoma cell line |
| cells | |
| HeLa, adherent cells | human cervical carcinoma cell line |
| HS68 | human diploid fibroblasts from newborn foreskin, |
| | ATCC-number CRL-1635 |
| 293T | human embryonic kidney cells 293 (ATCC-number |
| | CRL-1573) additionally transformed with the large |
| | T-antigen of the SV-40 virus |
| MCF7 | human mammary gland epithelial cells adenovirus- |
| | transformed (ATCC-number HTB-22) |
| K562 | human CML derived cell line (ATCC number |
| | CCL-243) |

Molecular biological methods

Preparation of plasmid DNA

Plasmid DNA was prepared according to the manufacturer's instructions using kits from Qiagen, Hilden, Germany.

Directed in vitro mutagenesis

Directed mutagenesis was carried out according to the manufacturer's instructions using the QuickChange Site-Directed Mutagenesis Kit from Stratagene, Heidelberg, Germany.

Cell biological methods

Culturing human cell lines

Adherent cell lines (HeLa, 293T, HS68 and MCF7) were cultured in cell culture dishes of variable size in standard medium at 37° C., 5% CO₂ and 100% air humidity (Hera cell, cell culture incubator, Heraeus, Hanau). 1 mM sodium pyruvate was additionally added to the medium for HS68 cells. HeLa, MCF7 and 293T cells were passaged every three days before confluence was reached. For this the cells were firstly washed with PBS, detached from the culture dish with 10× trypsin/EDTA and sown out at a dilution of 1:3 to 1:6 on new culture dishes. Cell counts were in general determined by means of a Neubauer counting chamber. HS68 cells were cultured under contact inhibition for several days to several weeks during which the medium was replaced every three days. If required the cells were treated with trypsin as described and sown in new culture dishes at a dilution of 1:4 to 1:8. HeLa suspension cells were cultured in Joklik's medium at 37° C. in spinner flasks while string constantly. The cell density of the cultures was kept between 2.5×10⁵ and 10⁶ cells per ml by daily dilution with fresh medium.

Cell Cycle analysis by flow cytometry

The percentage of cells in the G1, S and G2/M phases of the cell cycle was determined by two dimensional flow cytometry. The DNA content was determined by staining DNA with the intercalating dye propidium iodide and quantifying the cell fractions with differing DNA content by flow cytometry. In addition, cells in S-phase were labelled with the nucleotide analog bromo 5-bromo-2′-deoxyuridine (BrdU), which is incorporated into newly synthesized DNA. To determine the population of cells in S-phase, cells were incubated in the presence of BrdU for 30 min immediately before harvesting: human chronic myelogenous leukaemia K562 cells were first cotransfected with equal amounts of wt p27 or p27 Y88F mutant expressing plasmids and a GFP-expressing vector in ratio 5:1 by electroporation. Forty-eight hours following transfection, cells were pulse labelled with BrdU and the cell pools were separated by flow cytometry. Selected cells with equal level of GFP expression were fixed and stained with a FITC-conjugated anti-BrdU antibody and propidium iodide according to manufacturer's instructions (Beckman Dickinson). Cells were analyzed on FACS (Beckman Dickinson) flow cytometer using the CellQuest software. Analysis of data was carried out by the ModFit program.

Biochemical methods:

Immunoprecipitation of proteins

Precipitations with specific antibodies were carried out in order to purify a certain protein from total cell extracts (pulse-chase labelling, in vitro translation preparations or interaction investigation) or in order to identify the proteins associated in vivo with the precipitated protein in coimmunoprecipitations. All steps were carried out at 4° C. or on ice using precooled solutions.

The specific antibody (1-3 μg) was bound to 20 μl 50% /v/v) protein-A-Sepharose or 20 μl 50% (v/v) protein-G-Sepharose (1 h on a overhead rotator). After washing twice in IP buffer the suspension was incubated for 1-3 h with the protein extract in order to form the antigen-antibody complexes. After washing again three times with IP buffer, the Sepharose beads were taken up in 25 μl 2×SDS sample buffer and analysed after 5 min boiling by SDS-PAGE and Western blot.

Preparation of antisera and affinity purification of antibodies

In order to prepare the antigen the respective proteins were expressed recombinantly in E. coli as a GST or His fusion and purified by diverse methods up to the highest possible homogeneity. The purified protein (1 mg/ml) was intensively emulsified for the first injection in a ratio of 1:1 with TiterMax Gold using a double cannula. 250 μg protein was injected subcutaneously to immunize rabbits and 500 μg protein was used for goats. After 6 weeks further injections were carried out at intervals of 14 days in which TiterMax Gold was replaced by Freund's adjuvant. The withdrawn blood was kept for 2 h at room temperature for dotting and was subsequently centrifuged for 15 min at 5500 rpm to obtain the serum. The serum was stored at −20° C. or used directly for the affinity purification of antibodies. For the affinity purification of the specific antibody, 2-4 mg of the antigen was coupled covalently to cyanogen bromide-activated Sepharose 4B according to the manufacturer's instructions and washed. The serum was diluted 1:1 with PBS and incubated overnight at 4° C. with the immobilized antigen on an overhead rotator. The Sepharose was washed five times with 0.5 M NaCl in PBS and transferred to a column. The antibody was eluted with 0.2 M acetic acid, 0.5 M NaCl. The column eluate was collected in 0.5 ml fractions and immediately neutralized with 100 μl M Tris, pH 9.0 per fraction. The individual fractions were tested by spectral photometry for their antibody content. The fractions containing antibody were pooled, dialysed against PBS and concentrated (UH 100/l ultracartridge apparatus, Schleicher & Schüll, Dassel). Gelatin and sodium azide was added to the antibody solution to a final concentration of 0.2% (w/v) and 0.1% (w/v). The quality of the purified antibody was tested in an immunoblot using a concentration titration of the antibody against the specific antigen and total cell extract of HeLa cells. Phospho-specific antibodies against p27 were raised after synthesis of peptides where the desired phospho-amino acid was incorporated into the peptide. For tyrosine residue 88 the peptide NH₃-CLPEF(Y—PO₄)YRPPR-COO— was synthetised, where (Y—PO₄) is phosphotyrosine. Similar antisera were raised against the other tyrosines in p21, p27 and p57. Peptides were coupled to ovalbumin and injected intoi mammals (rabbits. goats and mice). Polyclonal antisera were purified first by depleting of all antibodies that cross-reacted with the non-phosphorylated peptide and subsequently by affinity purification against the phosphopeptide.

Polyclonal antibodies specific for Y88-phosphorylated p27 were generated by immunizing rabbits with the ovalbumin-coupled peptide LPEFpYYRPPR. Polyclonal antisera were purifyed by passage on a column linked to the unphosphorylated peptide followed by a final purification on a column linked to the phosphorylated peptide. Antibodies against Y89-phosphorylated p27 were generated using the coupled peptide PEFYpYRPPRP and antibodies against Y89-Y89 phosphorylated p27 using the peptide LPEFpYpYRPPR. Monoclonal antibodies directed against Y88-phosphorylated p27 were generated using the same peptide in mice and generated using standart procedures.

2-Dimensional gel electrophoresis

All materials for isoelectric focussing were obtained from Amersham Pharmacia Biotech (Freiburg). The focussing was carried out in a cooled (20° C.) Multiphor II according to the manufacturer's instructions. Isoelectric focussing strips (Immobiline DryStrip NL 3-10 or pH 6-11) containing 0.1 mg-2 mg protein in 300 μg rehydration buffer (8 M urea, 2% (w/v) CHAPS, 2% (v/v) IPG buffer (pH 3-10 or pH 6-11), 18.5 mM DTT) were incubated overnight in a rehydration cassette covered with a layer of oil for the focussing. The strips were subsequently focussed in a Multiphor II with an increasing voltage gradient.

| | |
|---|---|
| Gradient | 1st phase 300 V, 30 min |
| | 2nd phase 600 V, 30 min |
| | 3rd phase 900 V, 30 min |
| | 4th phase 1200 V, 30 min |
| | 5th phase 1700 V, 30 min. |
| | 6th phase 2200 V, 30 min |
| | 7th phase 2700 V, 30 min |
| | 8th phase 3200 V, 30 min |
| | 9th phase 3500 V for 18.5 to 27 h |

They were focussed for a total of 70,000-100,000 V/h depending on the amount of protein. The strips were briefly rinsed with ddH₂O, frozen at −80° C. or used directly for the separation in the second dimension. For this the strips were incubated three times for 5 min in equilibration buffer I (6 M urea, 30% (v/v) glycerol, 2% (w/v) SDS, 50 mM Tris/HCl pH 8.8, 10 mg/ml DTT), again briefly rinsed with ddH₂O and incubated three times for 5 min in equilibration buffer II (identical to equilibration buffer I but containing 25 mg/ml iodoacetamide instead of DTT). The strips treated in this manner were briefly rinsed with ddH₂O, drained and poured over an SDS-PAGE separation gel (12%, 20×20 cm) with hot 0.5% (w/v) agarose, dissolved in SDS electrophoresis buffer with traces of bromophenol blue. Electrophoresis in the second dimension was carried out as described under section 3.4.1.2. The separated proteins were analysed by Coomassie or silver staining or by specific antibodies after SDS-PAGE and immunoblot.

Preparation of cell lysates

Suspension HeLa cells were used to prepare total cell lysates from tissue culture cells or adherent cells were detached from the substrate with trypsin, centrifuged for 2 min at 1500 rpm and washed twice with PBS. The cell pellet was resuspended in IP or RIPA buffer and lysed by ultrasound on ice (Labsonic U, ultrasonic homogenizer, B. Braun, Melsungen). Alternatively IP or RIPA buffer was added directly to tissue culture cells after washing twice with PBS and they were transferred into the buffer by scraping them off with a cell scraper. Also in this case they were lysed by ultrasonic treatment. The crude extract was subsequently centrifuged for 15 min at 13000 g and 4° C.

Metabolic labelling of tissue culture cells with [³²P] ortho-phosphate and thin layer chromatographic separation of phosphoamino acids

A 250 ml flask of adherent HeLa or MCF7 tissue culture cells was cultured in each case up to a confluence of 80% for the metabolic labelling. These were washed twice with warm TBS and once with phospholabel medium and subsequently cultured for a further 4 h with 0.5 mCi [³²P] ortho-phosphate per ml phospholabel medium. The tissue culture cells were washed three times with PBS, 1 ml IP buffer (containing 150 μM Na ortho-vanadate and 100 μM KF phosphatase inhibitors) was added and they were detached from the culture substrate with a cell scraper. The cell suspension was homogenized on ice using a G23 cannula, boiled for 10 min at 100° C., cooled for 10 min on ice and centrifuged for 10 min at 13000 g. The supernatant was incubated with 50 μl protein A-Sepharose (50%, (v/v)) for 1 h on an overhead rotator at 4° C. in order to bind unspecific proteins. The binding to the precoupled antibody was also carried out for 1 h at 4° C. The Sepharose beads were washed four times with IP buffer containing phosphatase inhibitors and taken up in 25 μl 2×SDS sample buffer, separated by SDS-PAGE and blotted. After amido black staining the PVDF membrane was exposed to a sensitive film (MS) for one to two days using an amplifying screen. The resulting radioactive signals were matched with the membrane stained with amido black and the radioactively labelled proteins were cut out of the PVDF membrane. In this process only two thirds of a band was cut out in each case in order, after exposing the membrane again, to check whether the cut out piece of membrane corresponds with the radioactive signal. An antibody detection was also carried out with the remaining membrane in order to allocate the cut out bands to the desired specific protein.

The cut out piece of membrane was rehydrated briefly in methanol, washed twice in ddH₂O and 25 μl 6 N HCl was added. The protein was hydrolysed for 50 min at 110° C. in an incubator. The reaction was stopped with 25 μl ddH₂O and centrifuged. The supernatant was transferred to a new 0.5 ml reaction vessel and dried in a MD8C membrane vacuum pump (Vakuubrand, Wertheim). The dried hydrolysate was resuspended in 3.5 μl TLC buffer I, 1 μl phosphoamino acid standard (phosphoserine, phosphothreonine and phosphotyrosine, 0.3 μg/ml of each, Sigma) was added and 0.25 μl fractions were applied in one point on a thin layer chromatography cellulose plate. The phosphoamino acids were separated electrophoretically in two dimensions using a HTLE-7000 apparatus (CBS Scientific, USA) according to the manufacturer's instructions. The first dimension was carried out for 35 min at pH 1.9 in TLC buffer I at 1.5 kV. The plate was subsequently dried for 20 min with a hairdryer and a second electrophoresis rotated by 90° was carried out with TLC buffer II for 30 min at pH 3.5 and 1.3 kV. The plate was dried at 60° C. and the phosphoamino acid standard was visualized with 0.5% ninhydrin solution in acetone at 80° C. The radioactive phosphoamino acids were detected by a one to three week exposition of the TLC plate with an amplifier screen against a sensitive MS X-ray film.

Determination of the half-life of a protein by metabolic labelling with [³⁵S] methionine and [³⁵S] cysteine (pulse-chase)

In order to analyse the half-life of p27 and derived mutants, a 15 cm cell culture dish of HeLa tissue culture cells (80% confluent) was transfected in each case with HA fusion constructs of p27, they were detached from the culture dish with trypsin 5 h after the transfection and divided in equal portions per construct among four fresh cell culture flasks (corresponding to the number of the planned times for the half-life determination of the protein) and the culture was continued overnight. The cells were washed once with PBS and cultured for 60 min with 5 ml Hunger medium per flask to reduce endogenous methionine and cysteine.

“Pro-mix L-[³⁵S] in vitro cell labelling mix” (Amersham) at a final activity of 0.05 mCi/ml [35S] methionine and [³⁵S] cysteine was subsequently added to the cells and they were metabolically labelled for 1 h. The radioactive medium was in each case replaced by 25 ml chase medium, the medium was immediately removed again in the case of the 0 h value and the cells were harvested. For the other times the labelled cells were cultured for a further 2 h, 4 h and 6 h and subsequently harvested. For this the cells were washed with PBS, 1 ml 10× trypsin was added and they were incubated for 2 min at 37° C. 10 ml chase medium was added to the detached cells, they were transferred to a 15 ml Falcon vessel, centrifuged for 2 min at 1500 rpm, resuspended in 1.5 ml ice-cold PBS and transferred to a 2 ml reaction vessel. After a renewed centrifugation (2 min, 1500 rpm) the cell pellet was frozen in liquid nitrogen and stored at −80° C.

The cells were lysed in 750 μl IP buffer in each case by homogenization with a G23 cannula on ice. The samples were centrifuged for 5 min at 13000 g and the protein content of the supernatant was determined with the “Micro BCA Protein Assay Reagent Kit”. The supernatant was transferred to a 1.5 ml reaction vessel, boiled for 10 min at 100° C., cooled for 10 min on ice and the precipitated proteins were pelleted by centrifugation (10 min, 13000 g). In each case exactly identical amounts of protein were incubated with identical amounts of protein G-Sepharose-bound HA antibody for 1 h at 4° C., washed three times with lysis buffer, centrifuged and taken up in 25 μl 2×SDS sample buffer. The samples and aliquots of the supernatants were separated by SDS-PAGE and subsequently analysed by Coomassie staining and auto-radiography. The [³⁵S] total incorporation and the half-life of p27 was quantified with a Fujifilm BAS-2500 Phosphoimager (Fujifilm, Düsseldorf).

Expression of recombinant proteins in E. coli

In order to express recombinant proteins the corresponding plasmid constructs were transformed in the E. coli strain BL21(DE3) and an individual colony was inoculated in 200 ml to 10 l Superbroth medium. Protein expression was induced with 1 mM IPTG in the exponential growth phase at an OD600 of 0.8. After 4 to 5 h at 20° C. (e.g. the kinases Abl^{KD}, Abl^{FD+SH3}), 30° C. (the isolated SH3 domains and CAKp1-GST) or 37° C. (all other proteins expressed in this study) the cells were centrifuged, washed once with PBS and directly processed further or stored at −20° C.

Native and denaturing affinity purification of GST and His fusion proteins, renaturation of proteins

In this study proteins were produced recombinantly in E. coli as GST or His fusion proteins and purified therefrom. Two basic methods were used to purify proteins. If the protein was present in a soluble form in E. coli, a native affinity purification of the protein was carried out by means of a nickel-chelate or Glutathione Sepharose matrix. If the recombinant protein was present in an insoluble form in bacteria (inclusion bodies), a denaturing purification with subsequent renaturation of the protein was carried out.

Cell lysis:

The bacterial pellet was taken up in resuspension buffer (200 mM NaCl, 25 mM Na phosphate pH 8.0, 2.5 mM β-mercaptoethanol, 1 mM PMSF, 10 μg/ml aprotinin, pepstatin A, leupeptin) and disrupted on ice by means of ultrasound (Labsonic U, ultrasonic homogenizer, B. Braun, Melsungen). As an alternative to the ultrasonic treatment the bacterial pellet was lysed with 3.5 mg/ml lysozyme for 30 min at room temperature in 150 mM NaCl, 50 mM Tris pH 8.0 in the case of particularly sensitive proteins (e.g. the kinases Abl^{KD}, AbI^{KD+SH3}).

Native purification of proteins by nickel-chelate of glutathione Sepharose affinity chromatography.

A native purification by nickel-chelate chromatography was used for Abl^{KD}, AbI^{KD+SH3}, p27 and for the shortened form of p27 and amino acid substitutions of p27, p57 and for Grb2-His. The disrupted bacterial cells were centrifuged for 35 min at 45000 rpm to separate the bacterial cell debris. Approximately 1 mg “ProBond Nickel-Chelating Resin” per 10 mg recombinant protein was added to the centrifugation supernatant and incubated for 1 h at 4° C. on an overhead rotator. The immobilized protein was washed three times with resuspension buffer (additionally containing 10 mM imidazole) and eluted with 300 mM imidazole. The eluate was dialysed, optionally concentrated (Centricon, centrifuge filter units, ultrafiltration with Amicon ultrafiltration cell) or, if a higher degree of purity of the proteins was necessary, subjected to further purification steps.

The purification of GST fusion proteins was carried out similarly by means of a glutathione Sepharose matrix. In this case the immobilized protein was eluted with 20 mM glutathione at pH 8.0. Glutathione purification was used for the isolated SH3 domains of the 41 signal transduction proteins and for the isolated domains of Grb2, for p27-GST, Grb2-GST, for CAKp1-GST and for GST itself.

Denaturing purification of proteins by nickel-chelate or glutathione Sepharose affinity chromatography:

In order to purify inclusion bodies (e.g. p21 and amino acid substitutions of p21, FBX3) the pellet was resuspended by ultrasonic treatment in 200 mM NaCl, 50 mM Tris/HCl pH 8.0, 10 mM EDTA and 0.5% (w/v) Triton-X-100 solution to solubilize the bacterial membranes, incubated for 15 min at room temperature on an overhead rotator and centrifuged for 15 min at 10000 rpm and room temperature. This procedure was repeated twice, the pellet was subsequently homogenized with ultrasound in resuspension buffer, washed and centrifuged for 15 min at 10000 rpm. The inclusion body pellet was dissolved overnight at room temperature in denaturation buffer (8 M urea, 0.1 M Na phosphate, pH 8.0) in order to denature the protein. In the case of very poorly soluble proteins (Src^{rec.}) the pellet was dissolved with 12 M urea at 50° C. for 4 h. The denatured protein solution was centrifuged at 10000 rpm for 15 min at room temperature. The supernatant was applied to a nickel-chelate matrix, preequilibrated with denaturing buffer and incubated for 1-2 h on an overhead rotator. The immobilized proteins were washed three times with denaturing buffer and eluted with 300 mM imidazole in denaturing buffer.

The purification of GST fusion proteins (cdc14 active and cdc14 inactive) by a Glutathione Sepharose matrix was carried out analogously. The concentration of the urea solution was adjusted to 4 M after the proteins dissolve. The fusion protein was eluted from the glutathione Sepharose matrix with 20 mM glutathione at pH 8.0.

Renaturation of affinity-purified denatured proteins:

Four different renaturation strategies were used to renature the denatured affinity-purified proteins depending on the respective protein. In the simplest case the eluted protein could be refolded by a step dialysis (p57 from inclusion bodies). For this the eluate was dialysed in a dialysis tube for 2 h in each case with a urea concentration which was reduced by 2 M in each step. The last dialysis step was against IP buffer. Alternatively the protein was left immobilized on the chelate or glutathione matrix, renatured there with the same urea solutions of decreasing concentration and subsequently eluted from the matrix (cdc14) with imidazole (300 mM, pH 8.0) or glutathione (20 mM, pH 8.0). In another renaturation strategy the denatured protein eluate was diluted to 15 times its original volume by the dropwise addition of IP buffer (using a membrane pump for 24 h) while stirring continuously. Precipitated proteins were centrifuged and the supernatant was dialysed again overnight against IP buffer in order to remove the remaining urea (Src). The case of proteins that are very difficult to renature (p21 and amino acid substitutions of p21) ion exchange chromatography with immobilized renaturation and subsequent elution of the renatured protein by a salt gradient was used (see section 3.4.10). If necessary, the renatured proteins were concentrated (Centricon, centrifuge filter units, ultrafiltration with Amicon ultrafiltration cell) or, if a higher degree of protein purity was required they were subjected to further purification steps.

Ion exchange chromatography and gel filtration

Ion exchange chromatography

Chromatography on anion or cation exchangers was used to purify or concentrate proteins (p27 and derived forms, p57). Protein solutions were used for this from previous native or denatured affinity purifications (p57 from inclusion bodies), (see 3.4.9). Ion exchange chromatography was also used to renature proteins (p21 and derived forms). The chromatographies were carried out using anion and cation exchanger columns from Amersham. The chromatography was carried out with an “Áktapurifier FPLC/HPLC” system using the column parameters (program UNICORN 3.00) specified by the manufacturer (Amersham Pharmacia Biotech, Freiburg) for the respective column. In order to purify native proteins, the protein solution was dialysed for 24 h against a buffer having a low salt content (25 mM NaCl, 25 mM Tris pH 8.0, 1 mM PMSF, 1 mM DTT; buffer A). The protein solution was loaded onto an ion exchanger column preequilibrated with buffer A and non-bound proteins were washed out with 10 column volumes of buffer A. The protein was eluted from the column with a gradient of buffer A to buffer B (0.5 M-1 M NaCl (depending on the protein), 25 mM Tris/HCl pH 8.0, 1 mM PMSF, 1 mM DTT) at a flow rate of 1 ml/min. The eluate was collected in 0.25 ml fractions and analysed after SDS-PAGE by Coomassie staining or in an immunoblot.

In order to renature proteins, the denatured protein was dialysed for 24 h against denaturation buffer (8 M urea, 25 mM NaCl, 25 mM Tris, pH 8.0) and applied to a column preequilibrated with denaturing buffer. The column was washed with 10 volumes of denaturing buffer to remove unbound protein. The protein bound to the column matrix was renatured with a very slow gradient (0.1 ml/min flow rate) of denaturing buffer to buffer A over 24 h. The protein was eluted with a second gradient of buffer A to buffer B. The fractions of the desired protein were pooled, concentrated and frozen or optionally further purified.

Gel filtration:

Further purifications of proteins after an affinity and ion exchange chromatography were carried out by gel filtration of the protein sample (p27 and derived forms, p21 and p57). In addition analytical gel filtration was used to analyse the composition of a protein complex and to estimate the size of proteins in solution (CDK2/cyclin A; CDK2/cyclin A/p27; p27-Grb2). Gel filtrations were carried out with prepacked gel filtration columns (Amersham) using the FPLC/HPLC-Áktapurifier system. A protein solution was applied to a column prequilibrated with elution buffer (150 mM NaCl, 50 mM Tris pH 7.5, 1 mM PMSF, 1 mM DTT) and fractionated according to size in an isocratic elution over 1.5 column volumes.

The column eluate was collected in 0.25 ml-5 ml fractions and analysed after SDS-PAGE by Coomassie staining or in an immunoblot

Purification of Cip/Kip proteins by boiling and stabilizing proteins

A specific purification strategy for Cip/Kip proteins is based on the thermal stability of inhibitor proteins. Mammalian cell extracts or protein extracts from bacterial expressions of the recombinant inhibitors were boiled for 10 min at 100° C., cooled for 10 min on ice and separated from the precipitated protein impurities for 35 min at 45000 rpm. The resulting protein solution was used directly for investigations or the desired was further purified.

The proteins purified with the described methods (3.4.8-3.4.11) were usually immediately frozen in liquid nitrogen and stored at −80° C. In the case of very unstable proteins, the proteins were stabilized with stabilizing reagents (1-10% (w/v) glycerol (AblKD, AbIKD+SH3, CDK2/cyclin A), with 1 mg/ml BSA (cdc14, Srcrec.) or with 100 mM arginine for p21 and derived forms). In addition proteins were stabilized by varying the salt concentrations or the pH of the storage buffer.

Purification of recombinant proteins from insect cells

Active cyclin A-CDK2 from overexpressing insect cells was purified by nickel-chelate affinity purification and subsequent gel filtration. If not stated otherwise, all purification steps were carried out at 4° C. or on ice and with precooled solutions. Human cyclin A and an amino-terminal hexahistidine fusion protein of human CDK2 were expressed in Sf9 with the aid of recombinant baculoviruses. The baculovirus constructs originated from the laboratory of Dr. D. Morgan (University of California, San Francisco, USA).

The two insect cell pellets were resuspended in an equal volume of baculovirus lysis buffer (40 mM Na phosphate, 600 mM NaCl, 20% (w/v) glycerol, pH 8.0, 0.1 mM DTT, 2 mM PMSF, 2× complete without EDTA (Roche), 20 mM MgCl₂, 8 mM ATP) and disrupted with the aid of a dounce homogenizer (Braun, type L). The homogenisate for CDK2 was admixed with recombinant CAK1p protein (10 μg of a kinase activating kinase from yeast that can phosphorylate CDK2) and incubated for 45 min at 30° C. for phosphorylation and activation by the endogenous kinases of the insect cells and the recombinant CAK1p (Kaldis, 1999). The two homogenisates were combined and incubated for 1 h to form the CDK2/cyclin A complex. The homogenisate was centrifuged for 35 min at 35000 rpm, Probond nickel-chelaing resin was added to the supernatant and it was incubated for 2 h at 4° C. on an overhead rotator to immobilize the cyclin A-CDK2 complex. The complex was washed four times in IP buffer containing 10 mM imidazole and eluted from the chelate matrix with 300 mM imidazole in IP buffer. For further purification the eluate was concentrated further over a Superdex 200 pg HiLoad 26/60 gel filtration column (elution buffer: 150 mM NaCl, 50 mM Tris/HCl pH 7.5, 1 mM DTT, 0.5 mM PMSF). After analysis of the gel filtration fractions by SDS-PAGE and Coomassie staining, the fractions containing the cyclin A-CDK2 complex were frozen in liquid nitrogen and stored at −80° C.

In vitro phosphorylation of proteins

In vitro phosphorylation experiments were used to identify the substrates of kinases, to locate phosphorylatable amino acid residues in proteins and to test the activity of proteins which inhibit the kinases in a coupled kinasation inhibition experiment. In the case of phosphorylation of substrate proteins the phosphorylation of serine/threonine residues as well as the phosphorylation of tyrosine residues was examined.

Serine/threonine phosphorylations were carried out in kinasation buffer I. For this two mixtures were prepared per experiment which were combined for the kinasation:

| | | | |
|---|---|---|---|
| mix A: | 1 μl substrate protein | mix B: | 1 μl kinase (different |
| | (1-2 μg/μl) | | concentrations |
| | 1 μl ATP (25 μM final | | 1 μl kinasation buffer I (10 x) |
| | concentration) | | 0.5-1 μl [gamma-³²P]ATP |
| | add 10 μl ddH₂O | | (10 μCi/μl) |
| | | | add 10 μl ddH₂O |

p21, p27 and derived forms thereof and histone H1 and pRb were used as substrate proteins. CDK2/cyclin A was used as the kinase.

The kinase reaction was carried out for 30 min at 30° C. and stopped by adding 20 μl 2×SDS sample buffer. The phosphorylated proteins were separated by SDS-PAGE and subsequently analysed by autoradiography. The activity of kinase inhibitors (p21, p27, p57 and forms derived therefrom) were tested in an analogous manner. In this case the inhibitor protein (1 μl) was added to mix A. In order to determine the Km of the inhibitor different amounts of the inhibitor protein (0.1 ng-1 μg) were titrated against a constant amount of a kinase. Tyrosine phosphorylations were carried out in 20 μl kinasation buffer II. 0.5-2.5 μl kinase (Abl, Abl^{KD}, Abl^{KD+SH3}, Src^{Sf9}, Src^{rec.} and Lyn; various concentrations), 1 μl substrate protein (0.5-1 μg/μl, p21, p27, p57 and forms derived therefrom, and Sam68, RanGAP1 and BSA), ATP (50 μM final concentration) and 0.5-1 μl [γ-³²P] ATP (10 μCi/μl) and 1 μl BSA (2 μg/μl) were incubated for 2 h at 30° C. The kinasation was stopped with 20 μl 2×SDS sample buffer and the phosphorylated proteins were separated by SDS-PAGE and detected by autoradiography.

Serine/threonine and tyrosine kinasations were also carried out in an immobilized form. For this either the kinase or the substrate was bound to glutathione or nickel-chelate Sepharose or one of the two components (substrate or kinase) was immunoprecipitated from the cell extract on protein A Sepharose using specific antibodies. The reaction itself was carried out under the conditions required for the respective phosphorylation (see above). For this 25 μl of the required kinase buffer was added to the components immobilized on the Sepharose matrix. The sample was mixed every 5 min in order to improve the intermixing of the immobilized kinasation reactions.

In order to quantify the radioactivity incorporated into a protein, the gel was dried after SDS-PAGE and Coomassie staining and the Cherenkov radiation of the cut out protein bands was measured. Alternatively the activity was determined by a Phosphoimager System (Fujifilm BAS-2500 Phosphoimager, Fujifilm, Düsseldorf).

Retroviral expression and pulse-chase analysis

The human p27 cDNA or a p27-Y88F mutant were cloned into the pBABE retroviral vector and verified by sequencing. Viral stocks were generated in ecotropic Phoenix packaging cells. Phoenix cells were seeded at 2×10⁶ cells/10 cm plate and transfected with retroviral constructs twenty-four hours after plating. Cells were cultured overnight, medium was changed and 4 ml of viral supernatant harvested at 12, 24, 36 and 48 hours afterwards. The supernatants were filtered through 0.45-μm syringe-mounted filters and supplemented with 8 □g/ml of Polybrene prior to infection.

Recipient mouse embryonal fibroblasts nullyzygous for p27 (p27−/− MEFs) were subjected to four rounds of infection at 12-16, 24, 36 and 48 hours post plating. The cells were seeded for pulse-chase experiments 2 days post the last infection.

For pulse-chase analysis, subconfluent cultures of p27−/− MEFs retrovirally transduced with p27 wt or p27 Y88F mutant were incubated for one hour in methionine-free, cysteine-free DMEM culture medium at 37 C. The medium was then changed, and cells were metabolically pulse-labeled with ³⁵S methionine/cysteine by addition of the Pro-mix (Amersham) at 0.05 mCi/ml. The cell layers were incubated in the labeling medium for an 1.5 hour, washed with PBS, and incubated for 0, 1, 2, 3 or 5 hours in DMEM supplemented with 0.1 mg/ml of cysteine and 0.1 mg/ml methionine (chase). Following the various chase periods, the cells were collected, lysed in immunoprecipitation buffer, boiled for 15 minutes and the resulting supernatants harvested. p27 protein was immunoprecipitated from equal amounts of protein (as determined by the Micro BCA Protein Assay Reagent Kit)) utilizing polyclonal anti-rabbit p27 antibodies (done C-19, SCBT). The resulting immunoprecipitates were resolved by SDS-PAGE, the gels dried, and the labeled p27 visualized by autoradiography, and scanned by Phosphorimager. The intensity of the bands was calculated upon densitometric analysis using Image Software, and is expressed as a fraction of the time point 0 hrs.

### Example—Results and Discussion

p27^{Kip1} controls cell proliferation by binding to and regulating the activity of cyclin-dependent kinases (CDKs). The CDK inhibitor accumulates upon diverse antimitogenic signals, where increased p27 can prevent CDK activation and progression through the cell cycle (Sherr, C., and Roberts, J. M., Genes Dev. 13 (1999) 1501-1512; Hengst, L., and Reed, S. I., Curr. Top. Microbiol. Immunol. 227 (1998) 25-41). During the cell cycle, p27 is abundant in G1 phase and becomes eliminated as cells progress into S-phase. This involves both, reduction in synthesis and increased degradation of the inhibitor (Hengst, L., and Reed, S. I., Science 271 (1996) 1861-1864; Pagano, M., et al., Science 269 (1995) 682-685). p27 is degraded via the ubiquitin-proteasome pathway (Bloom, J., and Pagano, M., Semin. Cancer Biol. 13 (2003) 41-47). Activation of this pathway at the G1/S transition requires preceding phosphorylation of p27 on a threonine residue (Thr-187) (Bloom, J., and Pagano, M., Semin. Cancer Biol. 13 (2003) 41-47). This threonine is in a sequence context that corresponds to a canonical CDK consensus site, and it was demonstrated that active CDK2/cyclin E can phosphorylate CDK/cyclin bound p27(Bloom, J., and Pagano, M., Semin. Cancer Biol. 13 (2003) 41-47). While free active CDK2 phosphorylates CDK-bound p27 efficiently, structural and biochemical studies suggest that CDK2 kinase bound to p27 will be unable to exert this phosphorylation, since p27 inactivates the kinase by misfolding of its catalytical cleft and displacement of ATP (Pavletich, N. P., J. Mol. Biol. 287 (1999) 821-828). As long as CDK inhibitors are in excess over CDK/cyclin complexes and prevent their activation, the mechanism how p27 degradation is initiated remains a conundrum.

Here, it is shown that p27 can be phosphorylated on a conserved tyrosine residue (Tyr-88) in its CDK binding domain. Although tyrosine phosphorylated p27 still associates with CDK/cyclin complexes, inactivation of the kinase by the inhibitor is impaired. Instead, bound p27 now becomes an efficient substrate for phosphorylation on its destabilising Thr-187 residue. This novel mechanism may initiate p27 degradation in the absence of free CDK2 kinase.

While searching for novel proteins that bind to the p27 protein, Grb2 was affinity purified with p27 from HeLa extracts. The C-terminal SH3 domain of Grb2 bound directly to a proline rich element of p27, an interaction that was independently observed by the groups of Robert J. Shaeff (Moeller, S. J., et al., Mol. Cell, Biol. 23 (2003) 3735-3752; and Jun-ya Kato (Sugiyama, Y., et al., J. Biol. Chem. 276 (2001) 12084-12090). It was speculated that p27 might also bind other SH3 domain proteins and therefore a number of different SH3 domains fused to GST were screened for p27 binding. While most domains failed to interact stably with the CDK inhibitor, the SH3 domain of the tyrosine kinase Lyn bound the CDK inhibitor protein in vitro. In proximity to the proline-rich SH3-binding domain of p27 are three tyrosine residues. Formation of a p27/Lyn complex in vivo was strongly enhanced if a mutant of p27 lacking all three tyrosines was expressed (Matthias Grimmler and Ludger Hengst, unpublished observations). These initial observations suggested that p27 might be a substrate of tyrosine kinases.

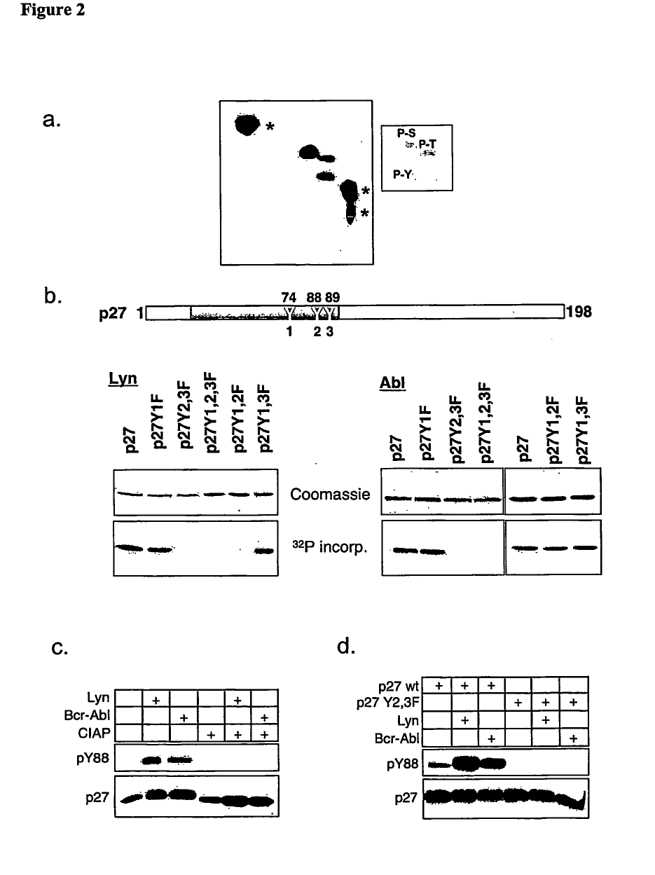

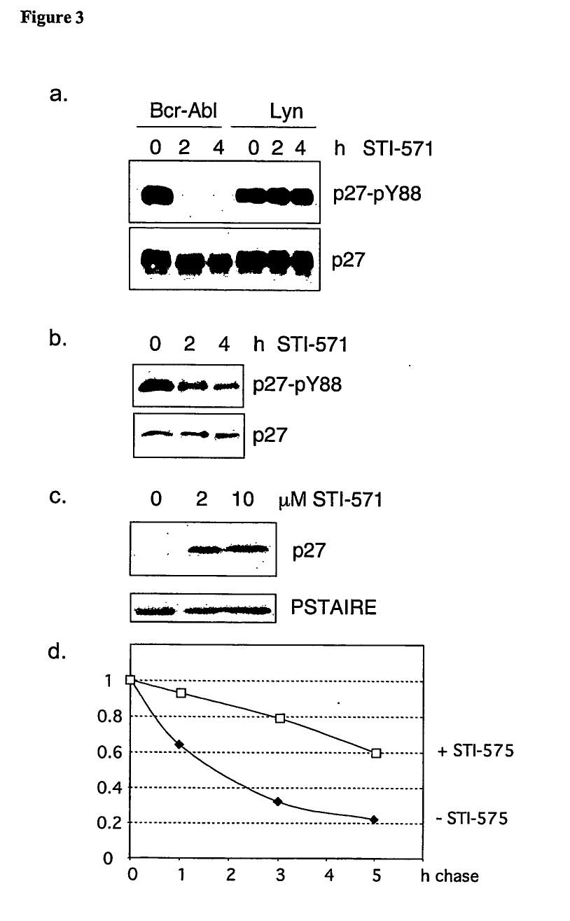

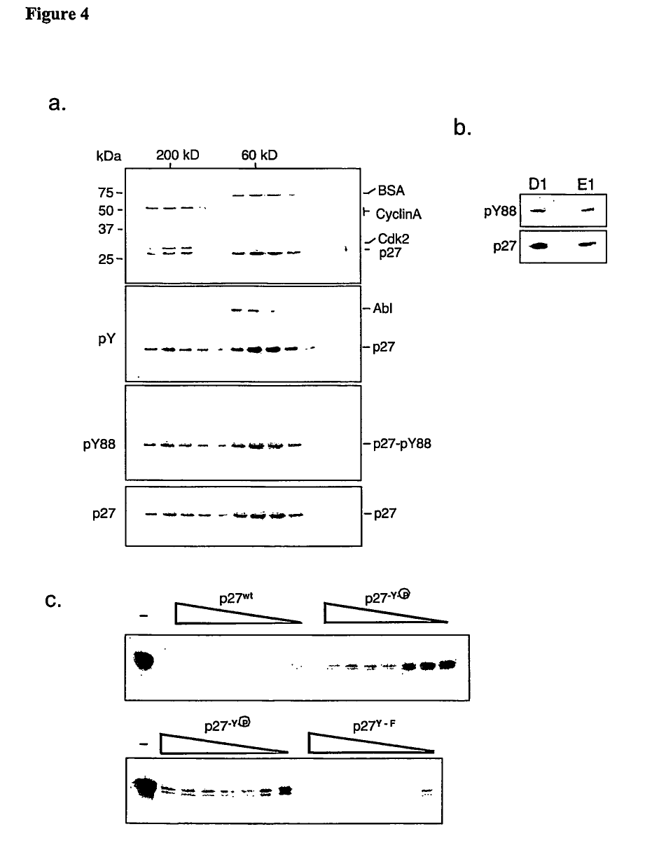

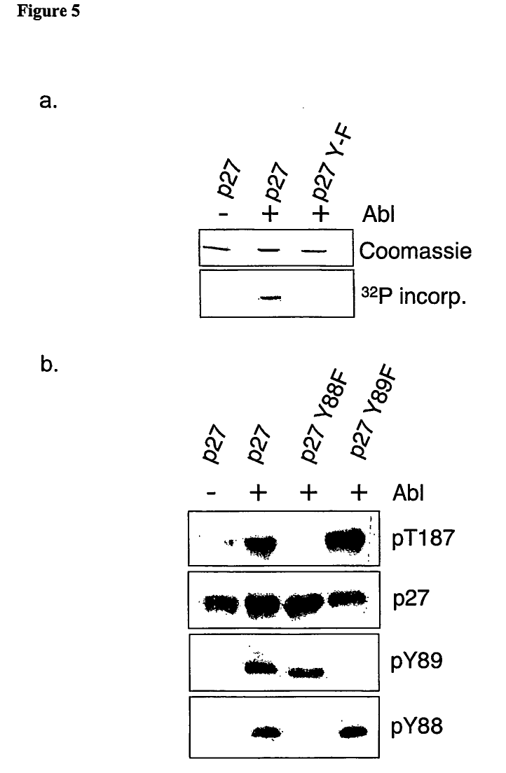

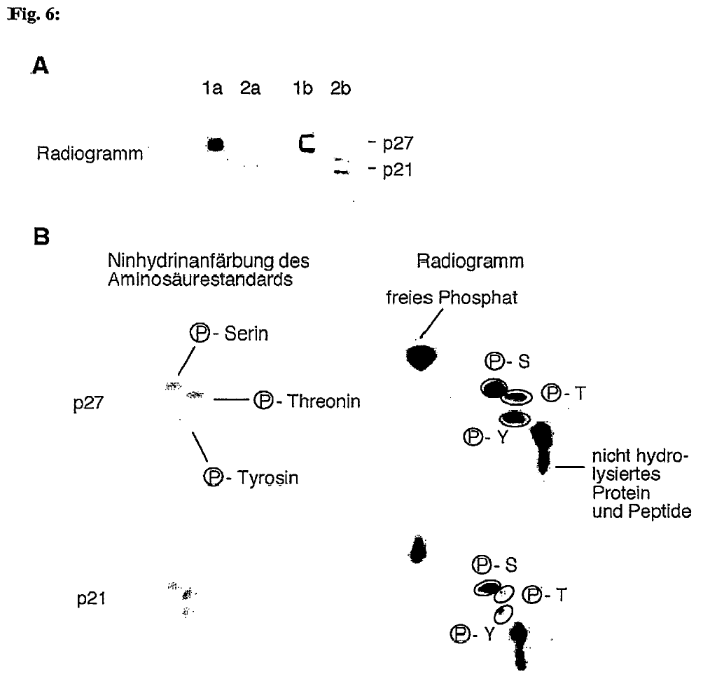

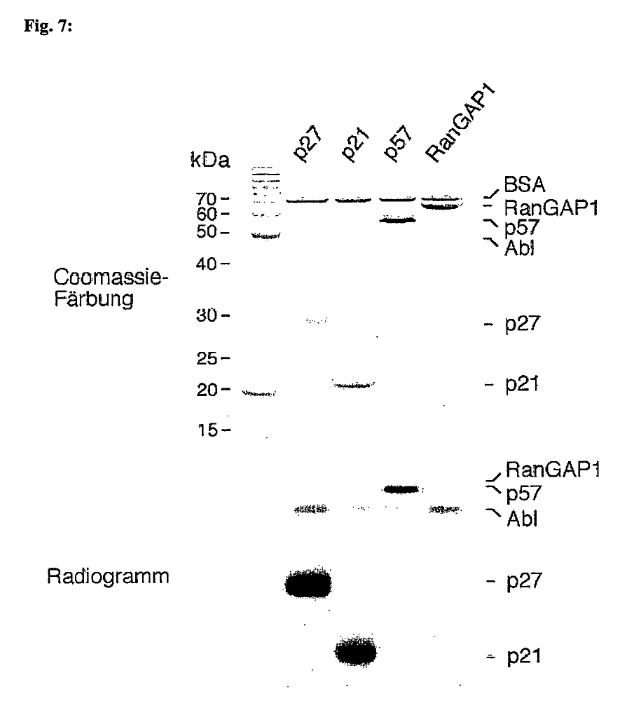

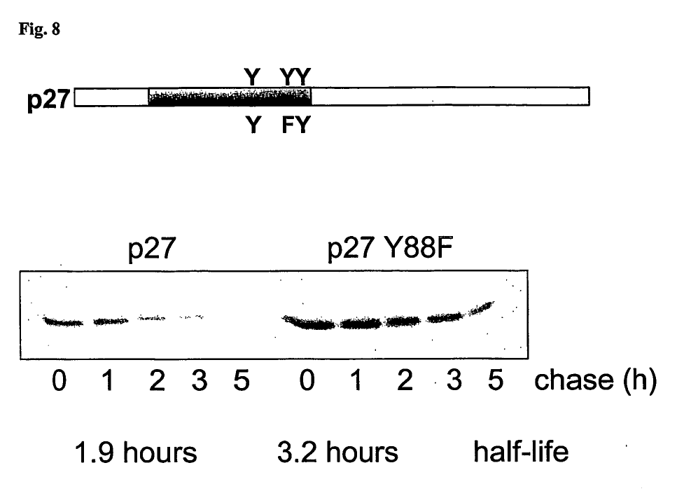

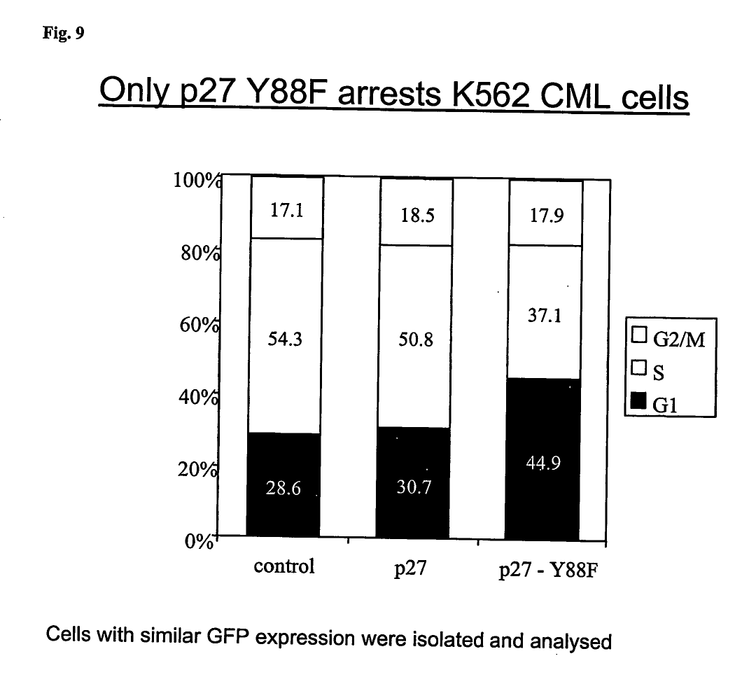

A number of phosphorylated isoforms of p27 have been reported (e.g. (Ishida, N., et al., J. Biol. Chem. 275 (2000) 25146-25154; Sheaff, R. J., et al., Genes Dev. 11 (1997) 1464-1478; and Muller, D., et al., Oncogene 15 (1997) 2561-2576), however exclusively serine and threonine phosphorylation of p27 have been described. To investigate whether p27 indeed can be phosphorylated on tyrosine residues in vivo, a phospho-amino acid analysis of immunoprecipitated overexpressed p27 protein from [³²P]-ortho-phosphate labelled HeLa cells was performed first. Besides phosphorylation on serine and threonine residues, a significant phosphorylation of tyrosine residues of p27 (FIG. 2a) was observed. Phosphatidyl-serine was most abundant, while incorporation of radioactive phosphate into tyrosine was comparable to that into threonine. The phosphatidyl-tyrosine was instable and easily lost during sample preparation, possibly explaining why it has escaped detection in previous studies. To investigate tyrosine phosphorylation of p21, we performed a phosphoamino-acid analysis of overexpressed p21 protein using the identical procedure and found that tyrosines of p21 can also be phosphorylated (FIG. 6). p21 contains only two tyrosine residues: one is highly conserved to tyrosine 88 of p27 and the second tyrosine is in the C-terminal PCNA binding domain of p21.

The non-receptor tyrosine kinases Src, Lyn and Abl were next tested for their ability to phosphorylate p27 in vitro. Indeed, all three kinases phosphorylated p27, however Lyn and Abl were more potent than Src (FIG. 2b and data not shown). In contrast to their ability to phosphorylate p27, the isolated SH3 domains of Src and Abl did not form a stable complex with p27 in vitro (data not shown). This suggests that stable binding of the kinase is no prerequisite for p27 tyrosine phosphorylation.

Next, it was aimed to identify tyrosine residues that can be modified in p27. The CDK inhibitor protein contains three tyrosine residues, which are all located within the N-terminal CDK inhibitory domain. First, all single tyrosine residues and combinations of double and triple tyrosines were replaced by phenylalanine (FIG. 2b). Only recombinant proteins containing the tyrosine residue 88 were efficiently phosphorylated by both kinases in vitro. The efficiency in phosphorylation for proteins lacking tyrosine 74 and tyrosine 89 was similar to that of wt p27. This suggested that tyrosine 88 is a phospho-acceptor site of p27. Lyn would not efficiently phosphorylate proteins in which tyrosine 88 was exchanged to phenylalanine, whereas Abl still phosphorylated proteins lacking tyrosine 88 but containing tyrosine residue 89, even though with reduced efficiency (FIG. 2b). The similar incorporation of phosphate in wt p27 and the mutant containing only tyrosine 88 suggests in average only one tyrosine per inhibitor molecule becomes phosphorylated. Tyrosine 88 is therefore a major phospho-acceptor site of p27 in vitro, while tyrosine 74 is not efficiently modified by Lyn and Abl kinases.

Tyrosine 88 is highly conserved, not only in the CDK inhibitory domains of all human Cip/Kip family members, but also in Cip/Kip related proteins of other eukaryotic species. We therefore investigated if p21 and p57 ara also substrate of tyrosine kinases and found that both proteins can be phosphorylated by Abl kinase (FIG. 7). While only the tyrosine homologous to tyrosine 88 of p27 is conserved in p57, p21 has two tyrosine residues. One is also homologous to tyrosine 88 of p27. Using phospho-specific antibodies against this tyrosine of p21, we could confirm that it can be phosphorylated in vivo (M. Pickel and L. Hengst, unpublished).

In order to investigate whether p27 is also a substrate for Lyn and Abl kinases in vivo, polyclonal phosphospecific antisera against phospho-tyrosine containing peptides (pY-88, pY-89 or both) were generated first. Consistent with our observation in vitro, phospho-tyrosine 88 could be detected in immunoprecipitates of p27 (FIG. 2c,d). If p27 was co-expressed with constitutive active Lyn or Bcr-Abl, significant increase in phosphorylation was observed (FIG., 2c, d). Expression of mutant p27 with tyrosine 88 and 89 exchanged to phenylalanine yielded no signal (FIG. 2d). To confirm specificity of the antibody, the immunoprecipitates were treated with phosphatase. A loss of the signal with the phosphotyrosine-88 specific antibodies was observed, while level of p27 remained unchanged (FIG. 2c). Thus p27 can indeed be phosphorylated on tyrosine-88 in vivo and overexpression of Bcr-Abl or constitutive active Lyn enhances this phosphorylation.

The Bcr-Abl oncogene is a product of the Philadelphia translocation t(9;22) (q34; q11) and causes chronic myelogenous and Bcr-Abl positive acute lymphoblastic leukaemia (CML, ALL) (Deininger, M. W., et al., Blood 96 (2000) 3343-3356). The transforming Bcr-Abl kinase is constitutively active and sufficient to induce and maintain leukemic transformation (Huettner, C. S., et al., Nat Genet 24 (2000) 57-69). The kinase can be inactivated by the small, highly selective synthetic tyrosine kinase inhibitor STI-571 (Wong, S., and Witte, O. N., Annu. Rev. Immunol. 22 (2004) 347-306). In order to investigate if tyrosine phosphorylation of p27 is directly dependent on Bcr-Abl kinase activity, STI-571 was added to 293T cells transfected with Bcr-Abl. Tyrosine-88 phosphorylation was barely detectable 2 hrs after addition of the kinase inhibitor, confirming that tyrosine-88 phosphorylation depends on active Bcr-Abl linase (FIG. 3a). To exclude indirect effects of the linase inhibitor, STI-571 was also added to cells transfected with Lyn. The Bcr-Abl kinase inhibitor had no effect on Tyr-88 phosphorylation by Lyn (FIG. 3a), supporting the hypothesis that p27 is a direct target of the Bcr-Abl oncogene.

Tyr-88 phosphorylation of p27 in a Bcr-Abl transformed tumor cell line was investigated next. K562 chronic myelogenous leukaemia cells express p210 Bcr-Abl kinase that is sensitive to inhibition with ST-571. Endogenous p27 isolated from K562 cells is phosphorylated on Tyr-88 (FIG. 3b). Addition of STI-571 abrogates Tyr-88 phosphorylation (FIG. 3b), suggesting that tyrosine phosphorylation of p27in these cells depends on the Bcr-Abl tyrosine kinase activity. Interestingly, a dramatic increase in p27 protein level after STI-571 inhibition of the Bcr-Abl kinase (FIG. 3c) was observed as had been observed previously in Bcr-Abl transformed cells (Gesbert, F., et al., J. Biol. Chem. 275 (2000) 39223-39230; Coutts, M., et al., Oncogene 19 (2000) 801-809; and Jonuleit, T., et al., Blood 96 (2000) 1933-1939). This increase in p27 protein correlated with an increase in p27 stability, as detected in pulse-chase experiments (FIG. 3d), suggesting that tyrosine phosphorylation may be involved in p27 degradation. However, it was not possible to investigate the stability of transfected mutant p27 proteins in K562 cells, because all overexpressed proteins had a significantly enhanced half-life compared to endogenous p27, likely due to the cell cycle arrest in G1 phase induced by the CDK inhibitors.

To investigate the mechanisms involved in p27 regulation in Bcr-Abl transformed leukaemia cells, the properties of Tyr-88 phosphorylated p27 were analysed next. Tyrosine-88 is located in the 3₁₀-helix of p27. Analysis by X-ray crystallography revealed that this helix inserts into the catalytic cleft of CDK2, where Tyr-88 becomes buried deep inside the deft and thereby occupies the purine binding pocket of the kinase. This prevents binding of ATP to CDK2⁶. Phosphorylation on this residue might therefore either prevent binding of p27 to CDK/cyclin complexes or induce a different mode of binding. To investigate whether tyrosine-phosphorylated p27 still binds the CDK complex, phosphorylated and unphosphorylated p27 were mixed in a ratio of 1:1. A large molar excess of the inhibitor mix was incubated with recombinant purified CDK2/cyclin A. If binding affinity of phosphorylated p27 were reduced, an enrichment of unphosphorylated p27 would be expected in kinase complexes. Complexes of p27 with cyclin A/CDK2 were separated from unbound inhibitor by gel filtration. Both forms of p27 bound to the kinase without significant differences (FIG. 4a), indicating that tyrosine phosphorylation does not prevent p27 from binding to the CDK complex. To support this observation, endogenous CDK/cyclin complexes from K562 human chronic myelogenous leukaemia cells were immunoprecipitated. CDK complexes precipitated with cyclin E1 or cyclin D1 specific antibodies contained endogenous p27 that is phosphorylated on Tyr-88 (FIG. 4b). Binding to the CDK/cyclin complex is therefore not impaired by Tyr-88 phosphorylation in vitro and in vivo.

This binding of the phosphorylated inhibitor to CDK complexes is consistent with the observation that a CDK inhibitory domain lacking the 3₁₀-helix (amino acids 28-79 of p27) still exerts some CDK inhibition in vitro (Polyak, K., et al., Cell 78 (1994) 59-66) and that mutant p27 deficient in stable CDK interaction still associates with CDK2 in vivo (Vlach, J., et al., Embo J. 16 (1997) 5334-5344). Recent kinetic studies of p27 binding to CDK/cyclin complexes have suggested that p27 binding follows a sequential mechanism. First, p27 binds to the cyclin subunit. Subsequently, the largely unstructured inhibitor is proposed to fold on the CDK2 subunit following a sequential binding on folding mechanisms (Lacy, E. R., et al., Nat. Struct. Mol. Biol. 11 (2004) 358-364). According to this model, insertion of the 3₁₀-helix into the catalytic cleft of CDK2 would be a final step in the binding on folding sequence. After Tyr-88 phosphorylation, the 3₁₀-helix of the inhibitor might bind to a different domain of CDK2, and thus alter the ability of p27 to inhibit the kinase. This might also provide an explanation for a number of reports that described active CDK2 kinase co-immunoprecipitating with p27 (e.g. Ciarallo, S., et al., Mol. Cell. Biol. 22 (2002) 2993-3002; and Radeva, G., et al., J. Biol. Chem. 272 (1997) 13937-13944). CDK inhibition of tyrosine-phosphorylated p27 was therefore compared with that of the unmodified protein. As shown in FIG. 4c, tyrosine phosphorylation severely impairs the ability of p27 to inhibit CDK2 linase activity. One important consequence of tyrosine phosphorylation is therefore a loss of efficient CDK inhibition.

p27 is inhibitor but also substrate of CDK2. CDK-mediated phosphorylation of p27on threonine 187 initiates its degradation by the ubiquitin proteasome pathway. If p27-bound CDKs remain active, the flexible inhibitor molecule might become phoshorylated while being bound to the kinase. It was therefore investigated if tyrosine phosphorylated p27 is a better substrate in CDK2 kinase assays. p27 was first phosphorylated with Abl kinase and repurified. An excess of phosphorylated or mock-treated p27 was incubated with CDK2/cyclin A to allow for saturation of the kinase complexes. p27/CDK2/cyclin A complexes were subsequently incubated with [³²P] γ-ATP, and phosphorylation of p27 by CDK2 was determined. As expected, p27 was poorly phosphorylated due to the absence of free kinase. By contrast, the tyrosine phosphorylated protein was a good substrate for CDK2 phosphorylation (FIG. 5a).

p27 contains one optimal CDK consensus motif surrounding threonine 187, which is phosphorylated by CDKs in vivo (Sheaff, R. J., et al., Genes Dev. 11 (1997) 1464-1478; Muller, D., et al., Oncogene 15 (1997) 2561-2576; Vlach, J., et al., Embo J. 16 (1997) 5334-5344; Toyoshima, H., and Hunter, T., Cell 78 (1994) 67-74; and Montagnoli, A., et al., Genes Dev. 13 (1999) 1181-1189). To investigate if the increased phosphorylation by CDK2 involves increased phosphorylation on this site, a phospho-threonine 187 specific antibody was used. Phosphorylation of Thr-187 was indeed strongly enhanced if Tyr-88 phosphorylated p27 was incubated in CDK2 kinase complexes (FIG. 5b). Importantly, the enhanced threonine phosphorylation is specific for phosphorylation of tyrosine 88, as a mutant phosphorylated on Tyr-89 is not a good substrate for CDK2-dependent phosphorylation (FIG. 5b). Increased phosphorylation on Thr-187 is known to stimulate SCF-Skp2-dependent degradation of p27. Therefore increased Thr-187 phosphorylation after Tyr-88 phosphorylation may well explain the enhanced degradation of p27 in Bcr-Abl transformed tumor cells. Consistent with our finding that altered binding of the inhibitor to a CDK/cyclin complex may trigger threonine phosphorylation and degradation, a similar mechanism albeit completely artificial has been demonstrated for a mutant of p27 lacking CDK interaction. The mutant did also associate with active CDK2 complexes and was degraded at an enhanced rate in vivo. Enhanced degradation of the mutant protein required the Thr-187 phosphoracceptor site (Vlach, J., et al., Embo J. 16 (1997) 5334-5344).

A number of links between p27 and other oncogenic pathways have been made, including the Ras, Myc and Akt-PKB pathways (Blain, S. W., et al., Cancer Cell 3 (2003) 111-115). The first direct link between p27 and oncogenic tyrosine kinases was identified herein. Phosphorylation of tyrosine 88 in p27 directly impairs its CDK inhibition, triggers CDK2-mediated threonine phosphorylation and thus initiates its degradation. This suggests that p27 Tyr-88 phosphorylation contributes to transformation by tyrosine kinases. Interestingly, STI-571 treatment of leukaemia patients frequently leads to relapse due to resistant clones. One of the STI-571-resistant cell lines overexpresses Lyn (Donato, N. J., et al., Blood 101 (2003) 690-698), a tyrosine kinase that can also phosphorylate Tyr-88 of p27.

p27 has been suggested as an independent prognostic marker in a number of human malignancies. However, there are a number of reports where p27 level do not correlate well with prognosis (Feakins, R. M., and Ghaffar, A. H., Hum. Pathol. 34 (2003) 385-390; and Alkarain, A., et al., Journal of Mammary Gland Biology and Neoplasia 9 (2004) 67). Enhanced tyrosine phosphorylation should contribute to the inactivation of p27 in these cells.

Our study predicts that a mutant of p27 that cannot be tyrosine phosphorylated would be stabilised. It might therefore act as a severely improved CDK inhibitory protein that is resistant to oncogenic transformation by tyrosine kinases. We therefore infected p27^{-/-} fibroblasts initially with Lyn kinase and subsequently a selected pool was infected with retroviruses that expressed either p27 or a mutant where tyrosine 88 was exchanged to phenylalanine. In agreement with our hypothesis, the half-life of the mutant p27 increased from 1.9 to 3.2 hours (FIG. 8).

To investigate if this mutant is more potent to arrest the cell cycle of Bcr-Abl transformed human tumor cells, K562 CML were transfected cells with GFP and either p27 or the mutant p27-Y88F. Whereas under conditions of moderate p27 expression the p27 protein was unable to alter the cell cycle of K562 cells, expression of p27-Y88F was increasing cells accumulating in G1 phase. At the same time, S-phase entry in these cells was impaired (FIG. 9). These data confirm our hypothesis that a p27-Y88F mutant is an improved CDK inhibitor in tumor cells. The mutant protein can impair proliferation under conditions where p27 fails to inhibit the cell cycle. The increased potency of the mutant p27-Y88F in arresting the cell cycle is likely due to improved CDK inhibition, lack of assembly of active CDK/cyclin D complexes and enhanced stability. These significant improved properties of the CDK inhibitory domain of p27 and the highly conserved domains of p21 and p57 may be essential for sustained inhibition of proliferation of tumor cells.

### LIST OF REFERENCES

Agrawal, D., et al., (1996) Mol Cell Biol, 16, 4327-4336
Alevizopoulos, K., et al., (1998) Embo J, 17, 5987-5997
Alkarain, A, et al., Journal of Mammary Gland Biology and Neoplasia 9, 67 (2004)
Altschul, S. F., et al., J. Mol. Biol. 215 (1990) 403-410
Altschul, S. F., et al., Nucleic Acids Res. 25 (1997) 3389-3402
Amann, E., et al., Gene 69 (1988) 301-315
Arkin, A. P., and Youvan, D. C., Proc. Natl. Acad. Sci. USA 89 (1992) 7811-7815
Asada, H., et al., (1999) J Exp Med, 189, 1383-1390
Ausubel et al., ed., Current Protocols in Molecular Biology, John Wiley & Sons, NY, 1992
Ausubel, F. M., et al., Current Protocols in Molecular Biology, (1994-1998)
Baldari, C., et al., EMBO J. 6 (1987) 229-234)
Baltensperger, K., et al., (1993) Science, 260, 1950-1952
Baneiji, J., et al., Cell 33 (1983) 729-740
Bartel, P., et al., Biotechniques 14 (1993) 920-924
Blain, S. W., et al., Cancer Cell 3, 111-5. (2003)
Bloom, J. & Pagano, M., Semin Cancer Biol 13, 41-7. (2003)
Boehm, M., et al, (2002) Embo J, 21, 3390-3401
Bonifadno, J. S., et al. (eds.), Current Protocols in Cell Biology, (2000) John Wiley & Sons, Inc.
Bouchard, C., et al., (1999) Embo J, 18, 5321-5333
Bourette, R. P., et al., (1998) Embo J, 17, 7273-7281
Boyle, W. J., et al, Methods Enzymol 201, 110-49. (1991)
Brandeis, M., and Hunt, T., Embo J. 15 (1996) 5280-5289
Breslauer, K. J., et al, Proc. Natl. Acad Sci. USA 83 (1986) 3746-3750
Brotherton, D. H., et al., (1998) Nature, 395, 244-250
Brown, J. P., et al., (1997) Science, 277, 831-834
Brugarolas, J., et al., (1995) Nature, 377, 552-557
Byrne, G. W., and Ruddle, F. H., Proc. Natl. Acad. Sci. USA 86 (1989) 5473-5477
Calame, K and Eaton, S., Adv. Immunol. 43 (1988) 235-275
Camper, S. A., and Tilghman, S. M., Genes Dev. 3 (1989) 537-546
Cantley, L. C. and Neel, B. G., (1999) Proc Natl Acad Sci. U S A, 96, 4240-4245
Carell et al., Angew. Chem. Int Ed. Engl. 33 (1994) 2061
Carnero, A., and Hannon, G. J., Curr. Top. Microbiol. Immunol. 227 (1998) 43-55
Carrano, A. C., et al., Nat. Cell. Biol. 1 (1999) 193-199
Carrell et al., Angew. Chem. Int. Ed. Erg. 33 (1994) 2059
Chamovitz, D. A. and Segal, D., (2001) EMBO Rep, 2, 96-101
Chan, F. K., et al., (1995) Mol Cell Biol, 15, 2682-2688
Chellappan, S. P., et al., (1998) Curr Top Microbiol Immunol, 227, 57-103
Chen, P., and Segil, N., Development 126 (1999) 1581-1590
Cheng, M., et al., EMBO J. 18 (1999) 1571-1583
Cho, C. Y., et al., Science 261 (1993) 1303-1305
Ciarallo, S., et al., (2002) Mol Cell Biol, 22, 2993-3002
Claret, F. X., et al., (1996) Nature, 383, 453-457
Clurman, B. E., et al., (1996) Genes Dev, 10, 1979-1990
Coats, S., et al., Science 272 (1996) 877-880
Coligan et al., ed., Current Protocols in Immunology, John Wiley & Sons, New York, 1994
Coutts, M., et al., Oncogene 19, 801-9. (2000)
Cruikshank, W. W., et al., J. Acquired Immune Deficiency Syndromes and Human Retrovirology 14 (1997) 193-203
Cull, M. G., et al., Proc. Natl. Acad. Sci. USA 89 (1992) 1865-1869
Current Protocols in Molecular Biology, John Wiley & Sons, NY, 1989, sections 6.3.1-6.3.6
Cwirla, S. E., et al., Proc. Natl. Acad. Sci. 87 (1990) 6378-6382
Deininger, M. W., et al., Blood 96, 3343-56. (2000)
Delagrave, S., et al., Prot. Eng. 6 (1993) 327-331
Deng, C., et al., (1995) Cell, 82, 675-684
Denhardt, D. T., (1999) In: Stein, G. S., et al. (eds.), The molecular basis of cell cycle and growth control, Wiley & Sons Inc., 225-304
Devlin, J. J., et al., Science 249 (1990) 404-406
DeWitt, S. H., et al., Proc. Natl. Acad. Sci. USA 90 (1993) 6909-6913
Diehl, J. A., et al., (1997) Genes Dev, 11, 957-972
Diehl, J A, et al., (1998) Genes Dev, 12, 3499-3511
Donato, N. J., et al., Blood 101, 690-8. (2003)
Draetta, G., et al., (1989) Cell, 56, 829-838
Du, M., et al, (1998) J. Biol Chem, 273, 24289-24292
Dudos, B., et al., (1991) Methods Enzymol, 201, 10-21
Dulic, V., et al., (1992) Science, 257, 1958-1961
Dulic, V., et al., (1994) Cell, 76, 1013-1023
Durand, B., et al., (1997) Embo J, 16, 306-317
Dyson, N., (1998) Genes Dev, 12, 2245-2262
Eckerskorn, C. and Lottspeich, F., (1990) Electrophoresis, 11, 554-561
Edlund, T., et al., Science 230 (1985) 912-916
Ekholm, S. V., and Reed, S. I., Curr. Opin. Cell Biol. 12 (2000) 676-684
Ekholm, S. V., et al., (2001) Mol Cell Biol, 21, 3256-3265
el-Deiry, W. S., et al., (1993) Cell, 75, 817-825
el-Deiry, W. S., et al., (1994) Cancer Res, 54, 1169-1174
EP-A 0 264 166
Erb, E., et al., Proc. Natl. Acad. Sd. USA 91 (1994) 11422-11426
Feakins, R. M. & Ghaffar, A. H., Hum Pathol 34, 385-90. (2003)
Felici, F., et al., Mol. Biol. 222 (1991) 301-310
Fero, M. L., et al., Cell 85 (1996) 733-744
Fero, M. L., et al., Nature 396 (1998) 177-180
Fodor, S. P., et al., Nature 364 (1993) 555-556
Fuchs, P., et al., Bio/Technology 9 (1991) 1370-1372
Furuno, N., et al., (1999) J Cell Biol, 147, 295-306
Gale, N. W., et al., (1993) Nature, 363, 88-92
Gallop, M. A., et al., J. Med. Chem. 37 (1994) 1233-1251
Gardner, L. B., et al., (2001) J Biol Chem, 276, 7919-7926
Gartel, A. L. and Tyner, A. L., (1999) Exp Cell Res, 246, 280-289
Gesbert, F., et al., J Biol Chem 275, 39223-30. (2000)
Girard, F., et al., (1991) Cell, 67, 1169-1179
Goeddel, Methods in Enzymology: Gene Expression Technology, Vol. 185, Academic Press, San Diego, Calif., 1991
Gottesman, In: Gene Expression Technology: Methods in Enzymology, Vol. 185, Academic Pres, San Diego, Calif., 1990, pp. 119-128
Graff, J. R., et al., (2000) J Biol Chem, 275, 24500-24505
Griffiths, A. D., et al., EMBO J. 12 (1993) 725-734
Gu, Y., et al., (1993) Nature, 366, 707-710
Guo, K., et al., (1995) Mol Cell Biol, 15, 3823-3829
Hage, D. S., and Tweed, S. A., J. Chromatogr. B. Biomed. Sci. Appl. 699 (1997) 499-525
Hagting, A., et al., (1999) Curr Biol, 9, 680-689
Halevy, O., et al., (1995) Science, 267, 1018-1021
Hamaguchi, T., et al., (1995) FEBS Lett, 372, 54-58
Hannon, G. J. and Beach, D., (1994) Nature, 371, 257-261
Hara, T., et al., J. Biol. Chem. 276 (2001) 48937-48943
Harder, K. W., et al., (2001) Immunity, 15, 603-615
Harlow and Lane, Antibodies: Laboratory Manual, Cold Spring Harbor Laboratory Press, Cold Spring Harbor, N.Y., 1988
Harper, J. W., et al., (1993) Cell, 75, 805-816
Harper, J. W., et al., (1995) Mol Biol Cell, 6, 387-400
Hartl, F. U., (1996) Nature, 381, 571-579
Hay, B. N., et al., Hum. Antibod. Hybridomas 3 (1992) 81-85
Heegaard, N. H., J. Mol. Recognit. 11 (1998) 141-148
Helmbrecht, K., et al., (2000) Cell Prolif, 33, 341-365
Hengst, L., and Reed, S. I., Curr. Top. Microbiol. Immunol. 227 (1998) 25-41
Hengst, L., and Reed, S. I., Science 271 (1996) 1861-1864
Hengst, L., et al., (1998) Genes Dev, 12, 3882-3888
Hengst, L., et al., Proc. Natl. Acad. Sci. USA 91 (1994) 5291-5295
Hibbs, M. L. and Dunn, A. R., (1997) Int J Biochem Cell Biol, 29, 397-400
Hirai, H., et al., (1995) Mol Cell Biol, 15, 2672-2681
Hirama, T. and Koeffler, H. P., (1995) Blood, 86, 841-854
Hohfeld, J., et al., (2001) EMBO Rep, 2, 885-890
Houghten, R. A., et al., Biotechniques 13 (1992) 412-421)
Howard, A., and Pelc, S. R., Exp. Cell Res. 2 (1951) 178-187
Huettner, C. S., et al., Nat Genet 24, 57-60. (2000)
Huse, W. D., et al., Science 246 (1989) 1275-1281
Ike, Y., et al., Nucleic Acid Res. 11 (1983) 477-488
Ilyin, G. P., et al., (2000) Genomics, 67, 40-47
Inoue, T., et al., (1999) J Biol Chem, 274, 32309-32317
Ishida, N., et al., (2000) J Biol Chem, 275, 25146-25154
Itakura, K., et al., Annu. Rev. Biochem. 53 (1984) 323-356
Itakura, K., et al., Science 198 (1977) 1056-1063
Iwabuchi, K., et al., Oncogene 8 (1993) 1693-1696
Jansen-Durr, P., et al., (1993) Proc Natl Acad Sci U S A, 90, 3685-3689
Jiang, Y., et al., (2000) Proc Natl Acad Sci U S A, 97, 10538-10543
Johnson, D., et al., (1998) Oncogene, 16, 2017-2028
Jonuleit, T., et al., Blood 96, 1933-9. (2000)
Kaldis, P., (1999) Cell Mol Life Sci, 55, 284-296
Kamijo, T., et al., (1997) Cell, 91, 649-659
Karlin, S., and Altschul, S. F., Proc. Natl. Acad. Sci. USA 87 (1990) 2264-2268
Karlin, S., and Altschul, S. F., Proc. Natl. Acad. Sci. USA 90 (1993) 5873-5877
Kato, G. and Maeda, S., (1999) J Biochem (Tokyo), 126, 957-961
Kaufman, R. J., et al., EMBO J. 6 (1987) 187-193
Kawamata, N., et al., Cancer Res. 55 (1995) 2266-2269
Kessel, M., and Gruss, P., Science 249 (1990) 374-379
Keyomarsi, K., et al., (1991) Cancer Res, 51, 3602-3609
Kingston, R. E., Moore, D. D., Seidman, J. G., Smith, J A. and Struhl, K. (Hrsg.). John Wiley & Sons, Inc., Current Protocols in Protein Science (1995-1997), Coligan, J. E., Dunn, B. M., Ploegh, H. L., Speicher, D. W. und Wingfield, P. T. (Hrsg.), John Wiley & Sons, Inc.
Kipreos, E. T., et al., (1996) Cell, 85, 829-839
Kiyokawa, H., et al., (1996) Cell, 85, 721-732
Knoblich, J. A. and Lehner, C. F., (1993) Embo J, 12, 65-74
Knudson, A. G., Jr., Proc. Natl. Acad. Sci. USA 68 (1971) 820-823
Koepp, D. M., et al., (2001) Science, 294, 173-177
Kohda, D., et al., (1994) Structure, 2, 1029-1040
Kohler, G., and Milstein, C., Nature 256 (1975) 495-497
Krek, W., (1998) Curr Opin Genet Dev, 8, 36-42
Kriwacki, R. W., et al., (1997) J Chromatogr A, 777, 23-30
Kriwacki, R. W., et al., (1996) Proc Natl Acad Sci. U S A, 93, 11504-11509
Kurjan, J., and Herskowitz, I., Cell 30 (1982) 933-943
LaBaer, J., et al., Genes Dev. 11 (1997) 847-862
Lacy, E. R., et al., Nat Struct Mol Biol 11, 358-64. Epub 2004 Mar 14. (2004)
Laemmli, U.K, (1970) Nature, 227, 680-685
Lam, K. S., Anticancer Drug Des. 12 (1997) 145-167
Lam, K. S., et al., Nature 354 (1991) 82-84)
Lee, M. H., et al., (1995) Genes Dev, 9, 639-649
Levine, A. J., (1997) Cell, 88, 323-331
Li, J., et al., (1997) Proc Natl Acad Sd U S A, 94, 502-507
Li, N., et al., (1993) Nature, 363, 85-88
Li, S., et al., (2001) Embo J, 20, 6793-6804
Li, Y., et al, (1994) Oncogene, 9, 2261-2268
Linder, M. W., et al., Clin. Chem. 43 (1997) 254-266
Liu, M., et al, (1996) Genes Dev, 10, 142-153
Lockhart, D. J., et al., Nat. Biotechnol. 14 (1996) 1675-1680
Lowenheim, H., et al., Proc. Natl. Acad. Sci. USA 96 (1999) 4084-4088
Lowenstein, E. J., et al, (1992) Cell, 70, 431-442
Luckow, V. A., and Summers, M. D., Virology 170 (1989) 31-39
Lukas, C., et al., (1999) Nature, 401, 815-818
Lundberg, A. S., et al., (2000) Curr Opin Cell Biol, 12, 705-709
Macleod, K. F., et al., (1995) Genes Dev, 9, 935-944
Madura, K., et al., J. Biol. Chem. 268 (1993) 12046-12054
Mal, A., et al., (1996) Nature, 380, 262-265
Malek, N. P., et al., Nature 413 (2001) 323-327
Martin-Caballero, J., et al., (2001) Cancer Res, 61, 6234-6238
Matsuoka, S., et al., (1995) Genes Dev, 9, 650-662
Matsuoka, S., et al., (1996) Proc Natl Acad Sci U S A, 93, 3026-3030
Matsushime, H., et al., (1992) Cell, 71, 323-334
McGraw, R. A., et al., BioTechniques 8 (1990) 674-678
Millard, S. S., et al., J. Biol. Chem. 272 (1997) 7093-7098
Milner, N., et al., Nat. Biotechnol. 15 (1997) 537-541
Moeller, S. J., et al., Mol Cell Biol 23, 3735-52. (2003)
Molinari, M., et al., (2000) EMBO Rep, 1, 71-79
Montagnoli, A., et al., Genes Dev. 13 (1999) 1181-1189
Morgan, D. O., (1997) Annu Rev Cell Dev Biol, 13, 261-291
Morgan, D. O., et al., (1989) Cell, 57, 775-786
Morimoto, M., et al., Biochem. Biophys. Res. Commun. 270 (2000) 1093-1096
Morosetti, R., et al., Blood 86 (1995) 1924-1930
Mou, S. and Linnekin, D., (1999) Biochem J, 342, 163-170
Muise-Helmericks, R. C., et al., (1998) J Biol Chem, 273, 29864-29872
Muller, D., et al., (2000) Embo J, 19, 2168-2180
Muller, D., et al., Oncogene 15 (1997) 2561-2576
Murata, S., et al., (2001) EMBO Rep, 2, 1133-1138
Myers and Miller, (1988)
Nagahara, H., et al., Nat. Med. 4 (1998) 1449-1452
Nakamura, S., et al., (1999) Biochem Biophys Res Commun, 257, 340-343
Nakayama, K, et al., (1996) Cell, 85, 707-720
Nakayama, K., et al., Mol. Cell. Biol. 19 (1996) 1190-1201
Narang, Tetrahedron 39 (1983) 3
Nguyen, H., et al., Mol. Cell Biol. 19 (1999) 1190-1201
Noda, A., et al, (1994) Exp Cell Res, 211, 90-98
O'Hagan, R. C., et al., Genes Dev. 14 (2000) 2185-2191
Ohtsubo, M., et al, Mol. Cell. Biol. 15 (1995) 2612-2624
Pagano, M., et al., (1992) Embo J, 11, 961-971
Pagano, M., et al., (1995) Science, 269, 682-685
Pantoja, C. and Serrano, M., (1999) Oncogene, 18, 4974-4982
Pardee, A. B., (1974) Proc Natl Acad Sci U S A, 71, 1286-1290
Pardee, A. B., (1989) Science, 246, 603-608
Parker, S. B., et al., (1995) Science, 267, 1024-1027
Patton, E. E., et al., (1998) Trends Genet, 14, 236-243
Pavletich, N. P., J Mol Biol 287, 821-8. (1999)
Pearson, W. R., and Lipman, D. J., Proc. Natl. Acad. Sci. USA 85 (1988) 2444-2448
Perez-Roger, I., et al., (1997) Oncogene, 14, 2373-2381
Philipp, A., et al., (1994) Mol Cell Biol, 14, 4032-4043
Pietenpol, J. A., et al., Cancer Res. 55 (1995) 1206-1210
Pines, J. and Hunter, T., (1990) Nature, 346, 760-763
Pines, J. and Rieder, C. L., (2001) Nat Cell Biol, 3, E3-6
Pinkert, C. A. et al., Genes Dev. 1 (1987) 268-277
Planas-Silva, M. D., and Weinberg, R A., Curr. Opin. Cell Biol. 9 (1997) 768-772
Plattner, R., et al., (1999) Genes Dev, 13, 2400-2411
Polyak, K., et al., (1994a) Genes Dev, 8, 9-22
Polyak, K., et al., (1994b) Cell, 78, 59-66
Puri, P. L., et al., (1999) In: Stein, G. S., Baserga, R., Giordano, A. und Denhardt, D. T. (eds.), The molecular basis of cell cycle and growth control, Wiley & Sons Inc., 15-79
Qiu, M., et al., (1998) Biochem Biophys Res Commun, 253, 443-447
Queen, C. and Baltimore, D., Cell 33 (1983) 741-748
Quelle, D. E., et al., Genes Dev. 7 (1993) 1559-1571
Radeva, G. et al., J Biol Chem 272, 13937-44 (1997)
Reed, S. I., (1997) Cancer Surv, 29, 7-23
Resnitzky, D., and Reed, S. I., Mol. Cell. Biol. 15 (1995) 3463-3469
Resnitzky, D., et al., Mol. Cell, Biol. 15 (1995) 4347-4352
Resnitzky, D., et al., Mol. Cell. Biol. 14 (1994) 1669-1679
Reynisdottir, I., and Massague, J., Genes Dev. 11 (1997) 492-503
Rivard, N., et al., J. Biol. Chem. 271 (1996) 18337-18341
Rivas, G., and Minton, A. P., Trends Biochem. Sci. 18 (1993) 284-287
Rodier, G., et al., (2001) Embo J, 20, 6672-6682
Roginskaya, V., et al., (1999) Leukemia, 13, 855-861
Rossig, L., et al., (2001) Mol Cell Biol, 21, 5644-5657
Roussel, M. F., (1999) Oncogene, 18, 5311-5317
Rozakis-Adcock, M., et al., (1993) Nature, 363, 83-85
Russo, A. A., et al., (1996a) Nature, 382, 325-331
Russo, A. A., et al., (1996b) Nat Struct Biol, 3, 696-700
Russo, A. A., et al., (1998) Nature, 395, 237-243
Saha, P., et al., (1997) Mol Cell Biol, 17, 4338-4345
Sambrook, J., Fritsch, E. F. und Maniatis, T. (1989) Molecular Cloning: A Laboratory Manual. Cold Spring Harbor Laboratory Press, Cold Spring Harbor, N.Y.
Sanger, F., et al., (1977) Proc Natl Acad Sci. U S A, 74, 5463-5467
Schultz, L. D., et al., Gene 54 (1987) 113-123
Scott, J. K., and Smith, G. P., Science 249 (1990) 386-390
Scott, M. T., et al., (2000) J Biol Chem, 275, 11529-11537
Seed, B., Nature 329 (1987) 840-842)
Seoane, J., et al., (2001) Nat Cell Biol, 3, 400-408
Serrano, M., et al., (1996) Cell, 85, 27-37
Sheaff, R. J., et al., Genes Dev. 11 (1997) 1464-1478
Sherr, C. J. and Roberts, J. M., (1999) Genes Dev, 13, 1501-1512
Sherr, C. J., (1993) Cell, 73, 1059-1065
Sherr, C. J., Science 274 (1996) 1672-1677
Sjolander, S. and Urbaniczky, C., Anal. Chem. 63 (1991) 2338-2345
Skoinik, E. Y., et al., (1993) Embo J, 12, 1929-1936
Slingerland, J. M., et al., Mol. Cell. Biol. 14 (1994) 3683-3694
Slingerland, J., and Pagano, M., J. Cell. Physiol. 183 (2000) 10-17
Smith, D. B., and Johnson, K. S., Gene 67 (1988) 31-40
Smith, G. E., et al., Mol. Cell Biol. 3 (1983) 2156-2165)
Sonenberg, N. and Gingras, A. C., (1998) Curr Opin Cell Biol, 10, 268-275
Sparks, A. B., et al., (1996) Proc Natl Acad Sci U S A, 93, 1540-1544
Spirin, K. S., et al., (1996) Cancer Res, 56, 2400-2404
Spirin, K. S., et al., Mol. Cell. 7 (1996) 639-650
Spruck, C., et al., Mol. Cell. 7 (2001) 639-650
Steiner, P., et al., (1995) Embo J, 14, 4814-4826
Stokoe, D. and McCormick, F., (1997) Embo J, 16, 2384-2396
Strohmaier, H., et al., (2001) Nature, 413, 316-322
Studier et al., In: Gene Expression Technology: Methods in Enzymology, Vol. 85, Academic Press, San Diego, Calif., 1991, pp. 60-89
Sugiyama, Y., et al., (2001) J Biol Chem, 276, 12084-12090
Sun, J., et al., (2001) Circulation, 103, 2967-2972
Sutterluty, H., et al., Nat. Cell. Biol. 1 (1999) 207-214
Szabo, A., et al., Curr. Opin. Struct Biol. 5 (1995) 699-705
Takizawa, C. G. and Morgan, D. O., (2000) Curr Opin Cell Biol, 12, 658-665
Takuwa, N., et al., (1999) Mol Cell Biol, 19, 1346-1358
Tautz, D. and Renz, M., (1983) Anal Biochem, 132, 14-19
Tomoda, K., et al., (1999) Nature, 398, 160-165
Tomoda, K., et al., (2002) J Biol Chem, 277, 2302-2310
Toyoshima, F., et al., (1998) Embo J, 17, 2728-2735
Toyoshima, H. and Hunter, T., (1994) Cell, 78, 67-74
Tsai, L. H., et al., (1991) Nature, 353, 174-177
Tsvetkov, L. M., et al., Curr. Biol. 9 (1999) 661-664
Tyers, M. and Jorgensen, P., (2000) Curr Opin Genet Dev, 10, 54-64
U.S. Pat. No. 4,868,103
U.S. Pat. No. 4,873,316
U.S. Pat. No. 5,223,409
U.S. Pat. No. 5,283,317
U.S. Pat. No. 5,631,169
van der Geer, P. and Hunter, T., (1993) Embo J, 12, 5161-5172
van Ness, J., and Chen, L., Nucleic Acids Res. 19 (1991) 5143-5151
Verbeek, B. S., et al., (1997) J Pathol, 183, 195-203
Vidal, A., and Koff, A., Gene 247 (2000) 1-15
Vigo, E., et al., (1999) Mol Cell Biol, 19, 6379-6395
Vlach, J., et al., Embo J. 16 (1997) 5334-5344
Wada, K., et al., Nucleic Acids Res. 20 (1992) 2111-2118
Wang, Q. M., et al., (2000) J Cell Physiol, 183, 238-246
Wei, N., et al., (1998) Curr Biol, 8, 919-922
Weinert, T., (1997) Cancer Surv, 29, 109-132
Welch, P. J. and Wang, J. Y., (1993) Cell, 75, 779-790
Weng, L. P., et al., (2001) Hum Mol Genet, 10, 599-604
Winoto, A., and Baltimore, D., EMBO J. 8 (1989) 729-733
Winston, J. T., et al., (1999) Curr Biol, 9, 1180-1182
WO 90/02809
WO 91/17271
WO 92/01047
WO 92/09690
WO 92/15679
WO 92/18619
WO 92/20791
WO 93/01288
WO 94/10300
Won, K. A. and Reed, S. I., (1996) Embo J, 15, 4182-4193
Wong, S. & Witte, O. N., Annu Rev Immunol 22, 247-306. (2004)
Xaus, J., et al., (2001) Blood, 98, 2124-2133
Xiong, Y., et al., (1993) Nature, 366, 701-704
Yan, Y., et al., (1997) Genes Dev, 11, 973-983
Yang, J., et al., (1998) Genes Dev, 12, 2131-2143
Yang, W., et al., (2001) Oncogene, 20, 1688-1702
Yaroslavskiy, B. B., et al., (2001) Mol Med, 7, 49-58
Yuan, Z. M., et al., (1996) Oncogene, 13, 939-946
Zachariae, W. and Nasmyth, K., (1999) Genes Dev, 13, 2039-2058
Zeng Y., et al., Biochem. Biophys. Res. Commun. 274 (2000) 37-42
Zervos, A. S., et al., Cell 72 (1993) 223-232
Zhang, H., et al., (1993) Mol Biol Cell, 4, 897-906
Zhang, H., et al., (1994) Genes Dev, 8, 1750-1758
Zhang, P., et al., (1997) Nature, 387, 151-158
Zhang, P., et al., (1999) Genes Dev, 13, 213-224
Zheng, N., et al., (2002) Nature, 416, 703-709
Zieve, G. W., et al., (1980) Exp Cell Res, 126, 397-405
Zuckermann, R. N., et al., J. Med. Chem. 37 (1994) 2678-2685

1-42. (canceled)

43. A polypeptide selected from the group consisting of:
a) a polypeptide comprising the amino acid sequence SEQ ID NO: 2, 4 or 6 or a peptide fragment with a minimum length of 6 amino acids,
whereby in the polypeptide comprising the amino acid sequence SEQ ID NO: 2, the tyrosine residue at position 88 and/or the tyrosine residue at position 89 in SEQ ID NO: 2 is/are phosphorylated, or
whereby in the polypeptide comprising the amino acid sequence SEQ ID NO: 4, the tyrosine residue at position 77 in SEQ ID NO: 4 is phosphorylated, or
whereby in the polypeptide comprising the amino acid sequence SEQ ID NO: 6, the tyrosine residue at position 91 in SEQ ID NO: 6 is phosphorylated;
b) a polypeptide
comprising the amino acid sequence SEQ ID NO: 2, 4 or 6 or
comprising the amino acid residues 1 to 95 of SEQ ID NO: 2 or comprising the amino acid residues 1 to 85 of SEQ ID NO: 4 or comprising the amino acid residues 1 to 100 of SEQ ID NO: 6 or
comprising the amino acid residues 50 to 95 of SEQ ID NO: 2 or comprising the amino acid residues 38 to 85 of SEQ ID NO: 4 or comprising the amino acid residues 50 to 100 of SEQ ID NO: 6
characterized in that in the polypeptide derived from the polypeptide with the amino acid sequence SEQ ID NO: 2, the amino acid residue at position 88 and/or position 89 in SEQ ID NO: 2, or
in the polypeptide derived from the polypeptide with the amino acid sequence SEQ ID NO: 4, the amino acid residue at position 77 in SEQ ID NO: 4 or
in the polypeptide derived from the polypeptide with the amino acid sequence SEQ ID NO: 6, the amino acid residue at position 91 in SEQ ID NO: 6
is a non-phosphorylatable amino acid residue, preferably a phenylalanine residue;
or a peptide fragment with a minimum length of 6 amino acids of a polypeptide comprising the amino acid sequence SEQ ID NO: 2, 4 or 6, characterized in that in the peptide fragment of a polypeptide comprising the amino acid sequence SEQ ID NO: 2, the peptide fragment comprises at least one of the flanking amino acid residues of the amino acid residue at position 88 or 89 in SEQ ID NO: 2 and the residue at position 88 and/or the residue at position 89 in SEQ ID NO: 2 is a non-phosphorylatable amino acid residue, preferably a phenylalanine residue, or in the peptide fragment of a polypeptide comprising the amino acid sequence SEQ ID NO: 4, the peptide fragment comprises at least one of the flanking amino acid residues of the amino acid residue at position 77 in SEQ ID NO: 4 and the residue at position 77 in SEQ ID NO: 4 is a non-phosphorylatable amino acid residue, preferably a phenylalanine residue, or
in the peptide fragment of a polypeptide comprising the amino acid sequence SEQ ID NO: 6, the peptide fragment comprises at least one of the flanking amino acid residues of the amino acid residue at position 91 in SEQ ID NO: 6 and the residue at position 91 in SEQ ID NO: 6 is a non-phosphorylatable amino acid residue, preferably a phenylalanine residue; or
c) a variant or peptidomimetics thereof.

44. A polypeptide according to claim 43, item a), comprising the amino acid sequence SEQ ID NO: 2, whereby the tyrosine residues at position 88 and/or the tyrosine residue at position 89 in SEQ ID NO: 2 are phosphorylated and whereby the serine residue at position 10 and/or 12 and/or the threonine residue at position 157 and/or the threonine residue at position 187 in SEQ ID NO: 2 are phosphorylated.

45. A variant or peptidomimetics according to claim 43, item c), comprising the amino acid sequence SEQ ID NO: 2, whereby the tyrosine residues at position 88 and/or the tyrosine residue at position 89 in SEQ ID NO: 2 are phosphorylated and whereby the serine residue at position 10 and/or 12 and/or the threonine residue at position 157 and/or the threonine residue at position 187 in SEQ ID NO: 2 are phosphorylated.

46. A peptide fragment according to claim 43, item a), with a minimum length of 10 amino acids, preferably with a minimum length of 15 amino acids.

47. A variant or peptidomimetics according to claim 43, item c), with a minimum length of 10 amino acids, preferably with a minimum length of 15 amino acids.

48. A nucleic acid molecule encoding a polypeptide according to claim 43, item b).

49. The nucleic acid molecule of claim 48, being contained in a vector.

50. The nucleic acid molecule of claim 48, being contained in a virus particle.

51. The nucleic acid molecule of claim 48, being contained in a mammalian cell.

52. An isolated antibody which specifically binds to a polypeptide according to claim 43 and which has less than 10% cross reactivity with the corresponding non-phosphorylated polypeptide or corresponding non-phosphorylated peptide fragment.

53. The antibody of claim 52 wherein the antibody is a monoclonal antibody.

54. The antibody of claim 53, wherein the monoclonal antibody is produced by the hybridoma cell line Mab<p27kip>15 or Mab<p27kip>388.

55. The antibody of claim 52, wherein the antibody is a polyclonal antibody.

56. The hybridoma cell line Mab<p27kip>15 or Mab<p27kip>388 as deposited with the DSMZ.

57. A method for treating a patient, involving administering to a patient a polypeptide according to claim 43.

58. The method of claim 57, wherein said treating is the treatment of hyperproliferative disease.

59. The method of claim 58, wherein the hyperproliferative disease is a cancer.

60. A pharmaceutical composition comprising a polypeptide according to claim 43 and a pharmaceutically acceptable carrier.

61. A method of determining whether or not a human cancer cell containing patient sample has potential for tumor progression, the method comprising comparing:
a) the level of expression a polypeptide according to claim 43 in the patient sample; and
b) the normal level of expression the polypeptide or the peptide fragment in a sample from a control subject not afflicted with cancer;
and an at least 1.5 fold difference or a less than 0.75 fold difference between the level of expression of the polypeptide or the peptide fragment in the patient sample and the normal level of the polypeptide or the peptide fragment in the sample from a control subject not afflicted with cancer is an indication that the patient sample has potential for tumor progression.

62. The method of claim 61, wherein the presence or the level of expression of said polypeptide or said peptide fragment is detected using a reagent which specifically binds with said polypeptide or peptide fragment.

63. The method of claim 62, wherein the reagent is an antibody, an antibody derivative, and an antibody fragment.

64. The method of claim 61, wherein the human cancer cell is a breast cancer cell, a colorectal cancer cell or a leukaemia cell, preferably a Philadelphia chromosome positive leukaemia cell, more preferably a chronic myeloid leukaemia cell or an acute lymphoblastic leukaemia cell.

65. The method of claim 61, wherein the sample is a tissue sample, blood or blood derived cells, primary cell cultures from patients, stool, lymph or a tissue-associated fluid or urine.

66. A method of selecting a composition for inhibiting the progression of cancer in a patient, the method comprising:
a) providing a sample comprising cancer cells from the patient,
b) separately exposing aliquots of the sample in the presence of a plurality of test compositions;
c) comparing the level of expression of the polypeptide according to claim 43 in each of the aliquots; and
d) selecting one of the test compositions which alters the level of expression of the polypeptide in the aliquot containing that test composition, relative to other test compositions.

67. The method of claim 66, wherein the presence or the level of expression of said polypeptide or said peptide fragment is detected using a reagent which specifically binds with said polypeptide or peptide fragment.

68. The method of claim 67, wherein the reagent is an antibody, an antibody derivative, and an antibody fragment.

69. The method according to claim 66, wherein the human cancer cell is a breast cancer cell, a colorectal cancer cell or a leukaemia cell, preferably a Philadelphia chromosome positive leukaemia cell, more preferably a chronic myeloid leukaemia cell or an acute lymphoblastic leukaemia cell.

70. The method according to claim 66, wherein the sample is a tissue sample, blood or blood derived cells, primary cell cultures from patients, stool, lymph or a tissue-associated fluid or urine.

71. A method to predict which patients will respond to a tyrosine kinase inhibitor drug in patients with a disorder whose underlying pathology involves the discontrol of a tyrosine kinase comprising:
a) contacting a sample from a patient with an antibody according to claim 52;
b) determining the level of phosphorylated polypeptide bound by the antibody of step a);
c) comparing the level of phosphorylated polypeptide determined in step b) for the sample with the level of phosphorylated protein in a reference sample, thereby detecting the responsiveness to a tyrosine kinase inhibitor drug in patients with a disorder whose underlying pathology involves the discontrol of a tyrosine kinase.

72. The method according to claim 71, wherein the reference sample is a sample from a patient who responds to the tyrosine kinase inhibitor drug.

73. The method according to claim 71, wherein the tyrosine kinase inhibitor is imatinib mesylate.

74. The method according to claim 71, wherein the patients with a disorder whose underlying pathology involves the discontrol of a tyrosine kinase are cancer patients.

75. The method according to claim 74, wherein the cancer patients are Philadelphia chromosome positive leukaemia patients preferably, chronic myeloid leukaemia patients or acute lymphoblastic leukaemia patients.

76. A method of deriving a candidate agent, said method comprising:
a) contacting a sample containing cancer cells, with said candidate agent;
b) determining the level of expression of the polypeptide according to claim 43 in the sample contacted with the candidate agent and determining the level of expression of the polypeptide in a sample not contacted with the candidate agent;
c) observing the effect of the candidate agent by comparing the level of expression of the polypeptide or the peptide fragment in the sample contacted with the candidate agent and the level of the polypeptide or the peptide fragment in the sample not contacted with the candidate agent;
d) deriving said agent from said observed effect;
wherein an at least 1.5 fold difference or a less than 0.75 fold difference between the level of expression of the polypeptide or the peptide fragment in the sample contacted with the candidate agent and the level of expression of the polypeptide or the peptide fragment in the sample not contacted with the candidate agent is an indication of an effect of the candidate agent.

77. The method according to claim 76, wherein said candidate agent is a candidate inhibitory agent.

78. The method according to claim 76, wherein said candidate agent is a candidate enhancing agent.
